(19) United States
(12) Patent Application Publication
(10) Pub. No.: US 2014/0249139 A1
(43) Pub. Date: Sep. 4, 2014

(54) Quinazoline Derivatives

(71) Applicants: Pascal Furet, Thann (FR); Christina Hebach, Muenchenstein (CH); Klemens Hogenauer, Oberwil (CH); Gregory Hollingworth, Kent (GB); Ian Lewis, Riehen (CH); Alexander Baxter Smith, Niffer (FR); Nicolas Solvermann, Village-Neuf (FR); Frederic Stauffer, Hesingue (FR); Romain Wolf, Schlierbach (FR); Frederic Zecri, Brookline, MA (US)

(72) Inventors: Pascal Furet, Thann (FR); Christina Hebach, Muenchenstein (CH); Klemens Hogenauer, Oberwil (CH); Gregory Hollingworth, Kent (GB); Ian Lewis, Riehen (CH); Alexander Baxter Smith, Niffer (FR); Nicolas Solvermann, Village-Neuf (FR); Frederic Stauffer, Hesingue (FR); Romain Wolf, Schlierbach (FR); Frederic Zecri, Brookline, MA (US)

(73) Assignee: NOVARTIS AG, Basel (US)

(21) Appl. No.: 14/352,055

(22) PCT Filed: Oct. 19, 2012
(86) PCT No.: PCT/IB2012/055745

§ 371 Date: Apr. 15, 2014

Related U.S. Application Data
(60) Provisional application No. 61/549,897, filed on Oct. 21, 2011.

(51) Int. Cl. C07D 401/04; C07D 413/14; C07D 401/14; C07D 487/08; C07D 403/04; C07D 239/74

(52) U.S. Cl. CPC C07D 401/04; C07D 403/04; C07D 239/74; C07D 401/14; C07D 487/08; C07D 413/14
USPC 514/218; 544/284; 514/252.17; 514/252.11; 544/117; 514/234.5; 544/58.2; 514/228.2; 540/575

(57) ABSTRACT

The invention relates to substituted quinazoline derivative of the formula (I), wherein A, X¹, X², X³, X⁴ and R⁵ are as defined in the description. Such compounds are suitable for the treatment of a disorder or disease which is mediated by the activity of the PI3K enzymes.

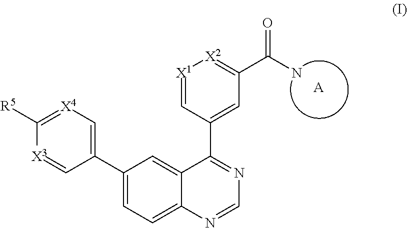

The invention relates to the preparation and use of new quinazoline derivatives as drug candidates in free form or in pharmaceutically acceptable salt form with valuable druglike properties, such as e.g. metabolic stability and suitable pharmacokinetics, form for the modulation, notably the inhibition of the activity or function of the phosphoinositide 3′ OH kinase family (hereinafter PI3K), suitably, the isoform PI3Kδ e.g. as indicated in in vitro and in vivo tests with selectivity of at least 10-fold, and more preferably at least 30-fold against the different paralogs PI3K α and β.

The selective inhibition of PI3Kδ is expected to avoid potential side effects mediated by PI3Kα and/or PI3Kδ such as inhibition of insulin signaling and inhibition of general cellular growth pathways.

Suitably, the invention relates to the treatment, either alone or in combination, with one or more other pharmacologically active compounds, of PI3K-related diseases including but not limited to autoimmune disorders, inflammatory diseases, allergic diseases, airway diseases, such as asthma and COPD, transplant rejection, cancers eg of hematopoietic origin or solid tumors.

In a first aspect, the invention relates to quinazoline compounds of the formula (I) and/or pharmaceutically acceptable salts and/or solvates thereof,

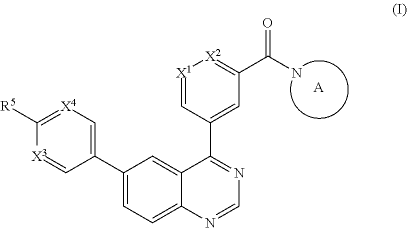

wherein

A is a saturated, 5-8 membered mono- or 6-12 membered bicyclic fused, bicyclic bridged or bicyclic spiro heterocyclic ring optionally containing 1-2 additional heteroatoms selected from N, O or S, wherein the heterocyclic ring is unsubstituted or substituted by 1-4 substituents selected from

hydroxy-

halo-

C₁-C₇-alkyl-

C₁-C₇-alkyl-carbonyl-

halo-C₁-C₇-alkyl-

halo-C₁-C₇-alkyl-carbonyl-

C₁-C₇-alkoxy-carbonyl-

oxo (O═);

X¹ and X² are CH, N, CR
wherein R is independently selected from
halogen-
halo-C₁-C₇-alkyl-
C₁-C₇-alkyl-
C₁-C₇-alkoxy-;

X³ is CH, N, CR³
wherein R³ is selected from
cyano-
nitro-
halogen-
halo-C₁-C₇-alkyl-
C₁-C₇-alkyl-
C₁-C₇-alkoxy-
C₁-C₁₀-cycloalkyl-oxy-
phenyl-oxy-
benzyl-oxy-
C₁-C₇-alkoxy-C₁-C₇-alkoxy-
carboxyl-
C₁-C₇-alkoxy-carbonyl-
amino-carbonyl-
N—C₁-C₇-alkyl-amino-carbonyl-
N,N-di-C₁-C₇-alkyl-amino-carbonyl-
amino-sulfonyl-
N—C₁-C₇-alkyl-amino-sulfonyl-
N,N-di-C₁-C₇-alkyl-amino-sulfonyl-
1-pyrrolidino-sulfonyl-
4-morpholino-sulfonyl-
C₁-C₇-alkyl-sulfonyl-
C₁-C₇-alkyl-sulfonyl-amino-;

X⁴ is CH, N, CR⁴
wherein R⁴ is selected from
F₃C—;

R⁵ is selected from

hydrogen-

halogen-

hydroxy-

C₁-C₇-alkyl-

C₁-C₇-alkoxy-

halo-C₁-C₇-alkyl-

halo-C₁-C₇-alkyl-oxy-

amino-

N—C₁-C₇-alkyl-amino-

N,N-di-C₁-C₇-alkyl-amino-

C₁-C₇-alkyl-carbonyl-

C₁-C₇-alkyl-carbonyl-amino-

amino-sulfonyl-

C₁-C₇-alkyl-sulfonyl-amino-

1-pyrrolidinyl-

1-piperazinyl-

with the proviso that, if X⁴ is CH, then R³ and R⁵ are not both methoxy.

Any formula given herein is intended to represent hydrates, solvates, and polymorphs of such compounds, and mixtures thereof.

As used herein, the term “a”, “an”, “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

The invention may be more fully appreciated by reference to the following description, including the following glossary of terms and the concluding examples. As used herein, the terms “including”, “containing” and “comprising” are used herein in their open, non-limiting sense. Where compounds of formula I are mentioned, this is meant to include also the tautomers and N-oxides of the compounds of formula I.

Where the plural form is used for compounds, salts, and the like, this is taken to mean also a single compound, salt, or the like.

The general terms used hereinbefore and hereinafter preferably have within the context of this disclosure the following meanings, unless otherwise indicated:

As used herein, the term “alkyl” refers to a fully saturated branched, including single or multiple branching, or unbranched hydrocarbon moiety having up to 20 carbon atoms. Unless otherwise provided, alkyl refers to hydrocarbon moieties having 1 to 16 carbon atoms, 1 to 10 carbon atoms, 1 to 7 carbon atoms, or 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl and the like. Typically, alkyl groups have 1-7, more preferably 1-4 carbons.

As used herein, the term “halo-alkyl” refers to an alkyl as defined herein, that is substituted by one or more halo groups as defined herein. The halo-alkyl can be mono-halo-alkyl, di-halo-alkyl or poly-halo-alkyl including per-halo-alkyl. A mono-halo-alkyl can have one iodo, bromo, chloro or fluoro within the alkyl group. Di-halo-alky and poly-halo-alkyl groups can have two or more of the same halo atoms or a combination of different halo groups within the alkyl. Typically the poly-halo-alkyl contains up to 12, or 10, or 8, or 6, or 4, or 3, or 2 halo groups. Non-limiting examples of halo-alkyl include fluoro-methyl, di-fluoro-methyl, tri-fluoro-methyl, chloro-methyl, di-chloro-methyl, tri-chloro-methyl, penta-fluoro-ethyl, hepta-fluoro-propyl, di-fluoro-chloro-methyl, di-chloro-fluoro-methyl, di-fluoro-ethyl, di-fluoro-propyl, di-chloro-ethyl and dichloro-propyl. A per-halo-alkyl refers to an alkyl having all hydrogen atoms replaced with halo atoms.

As used herein, the term “saturated heterocyclyl” for A refers to a ring system, for example a 5-, 6-, 7- or 8-membered monocyclic or 6-, 7-, 8-, 9-, 10-, 11-, or 12-membered bicyclic system and contains at least one heteroatom selected from N which is the point of attachment to the rest of the molecule. The heterocyclic group can be attached at a heteroatom or a carbon atom. The heterocyclic ring may contain 1-2 additional heteroatoms selected from N, O or S. The heterocyclyl can include fused or bridged rings as well as spirocyclic rings. Examples of heterocycles A include but are not limited to

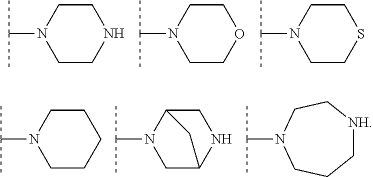

In another embodiment, examples of heterocycles A include but are not limited to

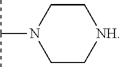

As used herein, the term “cycloalkyl” refers to saturated or partially unsaturated monocyclic, bicyclic or tricyclic hydrocarbon groups of 3-12 carbon atoms. Unless otherwise provided, cycloalkyl refers to cyclic hydrocarbon groups having between 3 and 10 ring carbon atoms or between 3 and 7 ring carbon atoms. Exemplary bicyclic hydrocarbon groups include octahydroindyl, decahydronaphthyl. Exemplary tricyclic hydrocarbon bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, bicyclo[2.2.2]octy. Exemplary tetracyclic hydrocarbon groups include adamantyl.

As used herein, the term “cycloalkyl” preferably refers to cyclopropyl, cyclopentyl or cyclohexyl.

As used herein, the term “oxy” refers to an —O— linking group.

As used herein, the term “carboxy” or “carboxyl” is —COOH.

As used herein, all substituents are written in a way to show the order of functional groups (groups) they are composed of. The functional groups are defined herein above. The point of their attachment is indicated with a hyphen (-) or an equal sign (=), as appropriate.

“Treatment” includes prophylactic (preventive) and therapeutic treatment as well as the delay of progression of a disease or disorder.

“Combination” refers to either a fixed combination in one dosage unit form, or a kit of parts for the combined administration where a compound of the formula (I) and a combination partner (e.g. an other drug as explained below, also referred to as “therapeutic agent” or “co-agent”) may be administered independently at the same time or separately within time intervals, especially where these time intervals allow that the combination partners show a cooperative, e.g. synergistic effect. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g. a compound of formula (I) and a combination partner, are both administered to a patient simultaneously in the form of a single entity or dosage. The term “non-fixed combination” means that the active ingredients, e.g. a compound of formula (I) and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

Various embodiments of the invention are described herein. It will be recognized that features specified in each embodiment may be combined with other specified features to provide further embodiments.

The invention further relates to pharmaceutically acceptable prodrugs of a compound of formula (I). Particularly, the present invention also relates to pro-drugs of a compound of formula I as defined herein that convert in vivo to the compound of formula I as such. Any reference to a compound of formula I is therefore to be understood as referring also to the corresponding pro-drugs of the compound of formula I, as appropriate and expedient.

The invention further relates to pharmaceutically acceptable metabolites of a compound of formula (I).

In one embodiment the invention provides a compound of the formula (I) and/or a pharmaceutically acceptable salt and/or a solvate thereof, wherein A is a saturated heterocycle selected from

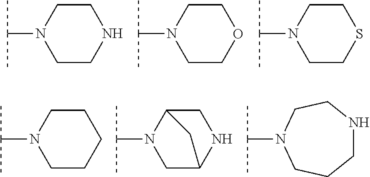

which is unsubstituted or substituted by 1-4 substituents selected from

hydroxy-

halo-

C₁-C₇-alkyl-

C₁-C₇-alkyl-carbonyl-

halo-C₁-C₇-alkyl-

halo-C₁-C₇-alkyl-carbonyl-

C₁-C₇-alkoxy-carbonyl-

oxo (O═).

In another embodiment the invention provides a compound of the formula (I) and/or a pharmaceutically acceptable salt and/or a solvate thereof, wherein

A is a saturated heterocycle selected from

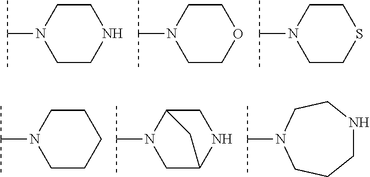

which is unsubstituted or substituted by 1-3 substituents selected from

hydroxy-

fluoro-

C₁-C₄-alkyl-

C₁-C₄-alkyl-carbonyl-

fluoro-C₁-C₄-alkyl-

oxo (O═).

In another embodiment the invention provides a compound of the formula (I) and/or a pharmaceutically acceptable salt and/or a solvate thereof, wherein

A is a saturated heterocycle selected from

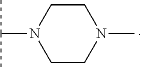

In another embodiment the invention provides a compound of the formula (I) and/or a pharmaceutically acceptable salt and/or a solvate thereof, wherein

A is a saturated heterocycle selected from

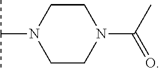

In another embodiment the invention provides a compound of the formula (I) and/or a pharmaceutically acceptable salt and/or a solvate thereof, wherein

X¹ is CH, N, CR¹
wherein R¹ is selected from
halogen-
halo-C₁-C₇-alkyl-
C₁-C₇-alkyl-
C₁-C₇-alkoxy-.

In another embodiment the invention provides a compound of the formula (I) and/or a pharmaceutically acceptable salt and/or a solvate thereof, wherein

X¹ is CH, N, CR¹
wherein R¹ is selected from
fluoro-
C₁-C₄-alkyl-
fluoro-C₁-C₄-alkyl-.

In another embodiment the invention provides a compound of the formula (I) and/or a pharmaceutically acceptable salt and/or a solvate thereof, wherein

X¹ is CH.

In another embodiment the invention provides a compound of the formula (I) and/or a pharmaceutically acceptable salt and/or a solvate thereof, wherein

X¹ is CR¹
wherein R¹ is selected from
fluoro-.

In another embodiment the invention provides a compound of the formula (I) and/or a pharmaceutically acceptable salt and/or a solvate thereof, wherein

X² is CH, N, CR²
wherein R² is selected from
C₁-C₇-alkyl-.

In another embodiment the invention provides a compound of the formula (I) and/or a pharmaceutically acceptable salt and/or a solvate thereof, wherein

X² is CH, N, CR²
wherein R² is selected from
C₁-C₄-alkyl-.

In another embodiment the invention provides a compound of the formula (I) and/or a pharmaceutically acceptable salt and/or a solvate thereof, wherein

X² is CH.

In another embodiment the invention provides a compound of the formula (I) and/or a pharmaceutically acceptable salt and/or a solvate thereof, wherein
X⁴ is N; and
R⁵ is selected from
C₁-C₇-alkyl-
C₁-C₇-alkoxy-
halo-C₁-C₇-alkyl-oxy-
amino-
N—C₁-C₇-alkyl-amino-
N,N-di-C₁-C₇-alkyl-amino-
1-pyrrolidinyl-
1-piperazinyl-.

In another embodiment the invention provides a compound of the formula (I) and/or a pharmaceutically acceptable salt and/or a solvate thereof, wherein
X⁴ is N; and
R⁵ is selected from
methoxy-.

In another embodiment the invention provides a compound of the formula (I) and/or a pharmaceutically acceptable salt and/or a solvate thereof, wherein
X⁴ is N;
R⁵ is selected from
C₁-C₇-alkyl-
C₁-C₇-alkoxy-
halo-C₁-C₇-alkyl-oxy-
amino-
N—C₁-C₇-alkyl-amino-
N,N-di-C₁-C₇-alkyl-amino-
1-pyrrolidinyl-
1-piperazinyl-; and
X³ is CH or CR³
   wherein R³ is selected from
   cyano-
   halogen-
   halo-C₁-C₇-alkyl-
   C₁-C₇-alkyl-.

In another embodiment the invention provides a compound of the formula (I) and/or a pharmaceutically acceptable salt and/or a solvate thereof, wherein
X⁴ is N;
R⁵ is selected from
C₁-C₄-alkyl-
C₁-C₄-alkoxy-
fluoro-C₁-C₄-alkyl-oxy-
amino-
N—C₁-C₄-alkyl-amino-
N,N-di-C₁-C₄-alkyl-amino-
1-pyrrolidinyl-
1-piperazinyl-; and
X³ is CH or CR³
   wherein R³ is selected from
   cyano-
   fluoro-
   chloro-
   fluoro-C₁-C₄-alkyl-
   C₁-C₄-alkyl-.

In another embodiment the invention provides a compound of the formula (I) and/or a pharmaceutically acceptable salt and/or a solvate thereof, wherein
X⁴ is N;
R⁵ is selected from
methoxy-; and
X³ is CH or CR³
   wherein R³ is selected from
   cyano-.

In another embodiment the invention provides a compound of the formula (I) and/or a pharmaceutically acceptable salt and/or a solvate thereof, wherein
X⁴ is N;
R⁵ is selected from
C₁-C₇-alkyl-
C₁-C₇-alkoxy-
halo-C₁-C₇-alkyl-oxy-
amino-
N—C₁-C₇-alkyl-amino-
N,N-di-C₁-C₇-alkyl-amino-
1-pyrrolidinyl-
1-piperazinyl-; and
X³ is N.

In another embodiment the invention provides a compound of the formula (I) and/or a pharmaceutically acceptable salt and/or a solvate thereof, wherein
X⁴ is N;
R⁵ is selected from
C₁-C₄-alkyl-
C₁-C₄-alkoxy-
fluoro-C₁-C₄-alkyl-oxy-
amino-
N—C₁-C₄-alkyl-amino-
N,N-di-C₁-C₇-alkyl-amino-
1-pyrrolidinyl-
1-piperazinyl-; and
X³ is N.

In another embodiment the invention provides a compound of the formula (I) and/or a pharmaceutically acceptable salt and/or a solvate thereof, wherein
X⁴ is N;
R⁵ is selected from
methoxy-; and
X³ is N.

In another embodiment the invention provides a compound of the formula (I) and/or a pharmaceutically acceptable salt and/or a solvate thereof, wherein
X⁴ is N;
R⁵ is selected from
hydrogen; and
X³ is CR³
wherein R³ is selected from
N,N-di-C₁-C₇-alkyl-amino-carbonyl-
N,N-di-C₁-C₇-alkyl-amino-sulfonyl-
1-pyrrolidino-sulfonyl-
4-morpholino-sulfonyl-
C₁-C₇-alkyl-sulfonyl-
C₁-C₇-alkyl-sulfonyl-amino-.

In another embodiment the invention provides a compound of the formula (I) and/or a pharmaceutically acceptable salt and/or a solvate thereof, wherein
X⁴ is N;
R⁵ is selected from
hydrogen; and
X³ is CR³
   wherein R³ is selected from
   C₁-C₄-alkyl-sulfonyl-.

In another embodiment the invention provides a compound of the formula (I) and/or a pharmaceutically acceptable salt and/or a solvate thereof, wherein
X⁴ is CH;
R⁵ is selected from
C₁-C₇-alkyl-
C₁-C₇-alkoxy-
halo-C₁-C₇-alkyl-oxy-
amino-
N—C₁-C₇-alkyl-amino-
N,N-di-C₁-C₇-alkyl-amino-; and
X³ is CR³
   wherein R³ is selected from
   cyano-
   halogen-
   halo-C₁-C₇-alkyl-
   C₁-C₇-alkyl-.

In another embodiment the invention provides a compound of the formula (I) and/or a pharmaceutically acceptable salt and/or a solvate thereof, wherein
X⁴ is CH;
R⁵ is selected from
C₁-C₄-alkyl-
C₁-C₄-alkoxy-
fluoro-C₁-C₇-alkyl-oxy-
amino-
N—C₁-C₄-alkyl-amino-
N,N-di-C₁-C₄-alkyl-amino-; and
X³ is CR³
   wherein R³ is selected from
   cyano-
   fluoro-
   chloro-
   fluoro-C₁-C₄-alkyl-
   C₁-C₄-alkyl-.

In another embodiment the invention provides a compound of the formula (I) and/or a pharmaceutically acceptable salt and/or a solvate thereof, wherein
X⁴ is CR⁴
   wherein R⁴ is selected from
   F₃C—;
R⁵ is selected from
amino-sulfonyl-
C₁-C₇-alkyl-sulfonyl-amino-; and
X³ is CH.

In another embodiment the invention provides a compound of the formula (I) and/or a pharmaceutically acceptable salt and/or a solvate thereof, wherein
X⁴ is CR⁴
   wherein R⁴ is selected from
   F₃C—;
R⁵ is selected from
hydrogen-; and
X³ is CH or CR³
   N,N-di-C₁-C₇-alkyl-amino-carbonyl-
   N,N-di-C₁-C₇-alkyl-amino-sulfonyl-
   1-pyrrolidino-sulfonyl-
   4-morpholino-sulfonyl-
   C₁-C₇-alkyl-sulfonyl-
   C₁-C₇-alkyl-sulfonyl-amino-.

In another embodiment the invention provides a compound of the formula (I) and/or a pharmaceutically acceptable salt and/or a solvate thereof, as described in the examples.

In another embodiment the invention provides a compound of the formula (I) and/or a pharmaceutically acceptable salt and/or a solvate thereof, wherein

A is a saturated heterocycle selected from

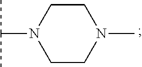

X¹ is CH;

X² is CH;

X⁴ is N;

R⁵ is selected from

methoxy-; and

X³ is N.

In another embodiment the invention provides a compound of the formula (I) and/or a pharmaceutically acceptable salt and/or a solvate thereof, wherein

A is a saturated heterocycle selected from

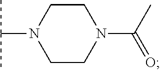

X¹ is CR¹
wherein R¹ is selected from fluoro-;

X² is CH;

X⁴ is N;

R⁵ is selected from

methoxy-; and

X³ is CH or CR³
wherein R³ is selected from
cyano-.

Compounds of the formula (I) may have different isomeric forms. As used herein, the term “an optical isomer” or “a stereoisomer” refers to any of the various stereo isomeric configurations which may exist for a given compound of the present invention and includes geometric isomers. It is understood that a substituent may be attached at a chiral center of a carbon atom. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-lngold-Prelog R—S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers or axes and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulformate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns 1 to 12 of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

For isolation or purification purposes it is also possible to use pharmaceutically unacceptable salts, for example picrates or perchlorates. For therapeutic use, only pharmaceutically acceptable salts or free compounds are employed.

In view of the close relationship between the novel compounds of the formula (I) in free form and those in the form of their salts, including those salts that can be used as intermediates, for example in the purification or identification of the novel compounds, any reference to the compounds or a compound of the formula (I) hereinbefore and hereinafter is to be understood as referring to the compound in free form and/or also to one or more salts thereof, as appropriate and expedient, as well as to one or more solvates, e.g. hydrates.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl, ¹²⁵I respectively. The invention includes various isotopically labeled compounds as defined herein, for example those into which radioactive isotopes, such as ³H, ¹³C, and ¹⁴C, are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Isotopically-labeled compounds of the formula (I) can generally be prepared by conventional techniques known to those skilled in the art or by processes analogous to those described in the accompanying Examples and Preparations using an appropriate isotopically-labeled reagents in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

Compounds of the invention, i.e. compounds of the formula (I) that contain groups capable of acting as donors and/or acceptors for hydrogen bonds may be capable of forming co-crystals with suitable co-crystal formers. These co-crystals may be prepared from compounds of the formula (I) by known co-crystal forming procedures. Such procedures include grinding, heating, co-subliming, co-melting, or contacting in solution compounds of the formula (I) with the co-crystal former under crystallization conditions and isolating co-crystals thereby formed. Suitable co-crystal formers include those described in WO 2004/078163. Hence the invention further provides co-crystals comprising a compound of the formula (I).

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated bonds may, if possible, be present in cis-(Z)- or trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Mixtures of isomers obtainable according to the invention can be separated in a manner known to those skilled in the art into the individual isomers; diastereoisomers can be separated, for example, by partitioning between polyphasic solvent mixtures, recrystallisation and/or chromatographic separation, for example over silica gel or by e.g. medium pressure liquid chromatography over a reversed phase column, and racemates can be separated, for example, by the formation of salts with optically pure salt-forming reagents and separation of the mixture of diastereoisomers so obtainable, for example by means of fractional crystallisation, or by chromatography over optically active column materials.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent. Compounds of the present invention are either obtained in the free form, as a salt thereof, or as prodrug derivatives thereof.

When both a basic group and an acid group are present in the same molecule, the compounds of the present invention may also form internal salts, e.g., zwitterionic molecules.

The present invention also provides pro-drugs of the compounds of the present invention that converts in vivo to the compounds of the present invention. A pro-drug is an active or inactive compound that is modified chemically through in vivo physiological action, such as hydrolysis, metabolism and the like, into a compound of this invention following administration of the prodrug to a subject. The suitability and techniques involved in making and using pro-drugs are well known by those skilled in the art. Prodrugs can be conceptually divided into two non-exclusive categories, bioprecursor prodrugs and carrier prodrugs. See The Practice of Medicinal Chemistry, Ch. 31-32 (Ed. Wermuth, Academic Press, San Diego, Calif., 2001). Generally, bioprecursor prodrugs are compounds, which are inactive or have low activity compared to the corresponding active drug compound, which contain one or more protective groups and are converted to an active form by metabolism or solvolysis. Both the active drug form and any released metabolic products should have acceptably low toxicity.

Carrier prodrugs are drug compounds that contain a transport moiety, e.g., that improve uptake and/or localized delivery to a site(s) of action. Desirably for such a carrier prodrug, the linkage between the drug moiety and the transport moiety is a covalent bond, the prodrug is inactive or less active than the drug compound, and any released transport moiety is acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of (a) hydroxyl groups with lipophilic carboxylic acids (e.g., a carboxylic acid having at least one lipophilic moiety), or (b) carboxylic acid groups with lipophilic alcohols (e.g., an alcohol having at least one lipophilic moiety, for example aliphatic alcohols).

Exemplary prodrugs are, e.g., esters of free carboxylic acids and S-acyl derivatives of thiols and O-acyl derivatives of alcohols or phenols, wherein acyl has a meaning as defined herein. Suitable prodrugs are often pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters, such as the omega-(amino, mono- or di-lower alkylamino, carboxy, lower alkoxycarbonyl)-lower alkyl esters, the alpha-(lower alkanoyloxy, lower alkoxycarbonyl or di-lower alkylaminocarbonyl)-lower alkyl esters, such as the pivaloyloxymethyl ester and the like conventionally used in the art. In addition, amines have been masked as arylcarbonyloxymethyl substituted derivatives which are cleaved by esterases in vivo releasing the free drug and formaldehyde (Bundgaard, J. Med. Chem. 2503 (1989)). Moreover, drugs containing an acidic NH group, such as imidazole, imide, indole and the like, have been masked with N-acyloxymethyl groups (Bundgaard, Design of Prodrugs, Elsevier (1985)). Hydroxy groups have been masked as esters and ethers. EP 039,051 (Sloan and Little) discloses Mannich-base hydroxamic acid prodrugs, their preparation and use.

Furthermore, the compounds of the present invention, including their salts, can also be obtained in the form of their hydrates, or include other solvents used for their crystallization. The invention relates in a second aspect to the manufacture of a compound of formula I. The compounds of formula I or salts thereof are prepared in accordance with processes known per se, though not previously described for the manufacture of the compounds of the formula I.

General Reaction Processes:

In one embodiment, the invention relates to a process for manufacturing a compound of formula I (Method A) comprising the step a of reacting a compound of formula II

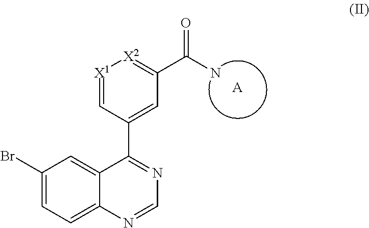

wherein the substituents are as defined above, with a compound of formula III,

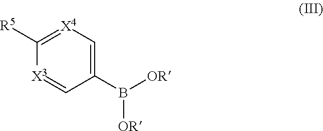

wherein the substituents are as defined above and —B(OR′)₂ represents a cyclic or acyclic boronic acid or boronic acid derivative, such as pinaccolato-boron, in the presence of a catalyst, such as a Pd(0) catalyst, e.g. Pd(PPh₃)₄, optionally in the presence of one or more reaction aids, such as a base, e.g. an aqueous base, optionally in the presence of one or more diluents, particularly polar solvents, e.g. acetonitrile. The reaction is stirred at a temperature of approximately 100-120° C. e.g. in a microwaves oven. The reaction may be carried out under an inert gas such as nitrogen or argon. This type of reaction is also known as Suzuki reaction, typical reaction conditions are known in the field and may applied to the present process;
wherein the compound of formula II is prepared comprising the step b of reacting a compound of formula IV

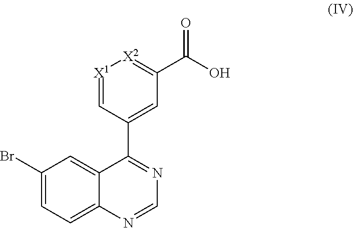

wherein the substituents are as defined above, with an amine of formula V,

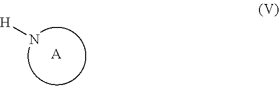

wherein the substituents are as defined above; under customary condensation conditions. The reaction is carried on by dissolving the carboxylic acid and the amine of formula V in a suitable solvent, for example halogenated hydrocarbon, such as methylene chloride, N,N-dimethylformamide, N,N-dimethylacetamide, N-2-methyl-pyrrolidone, methylene chloride, or a mixture of two or more such solvents, and by the addition of a suitable base, for example triethylamine, diisopropylethylamine (DIPEA) or N-methylmorpholine and a suitable coupling agent that forms a reactive derivative of the carboxylic acid in situ, for example (2-(1H-Benzotriazole-1-yl)-1,1,3,3-tetramethyluronium hexafluorophosphate (HBTU). The reaction mixture is stirred at a temperature of from approximately −20 to 50° C., such as from −5° C. to 30° C., e.g. at 0° C. to room temperature. The reaction may be carried out under an inert gas, e.g. nitrogen or argon;
wherein the compound of formula IV is prepared comprising the step c of saponifying a compound of formula VI

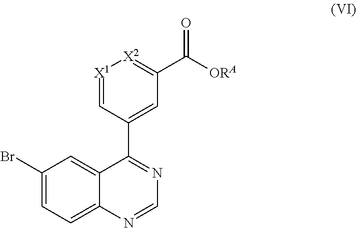

wherein the substituents are as defined above and R^{A} is a selected from C₁-C₇-alkyl. Saponification of the carboxylic ester is performed under customary saponification conditions, in the presence of an aqueous bases such as for example lithium hydroxide and a polar organic solvent such as for example dioxane. The reation is carried out at approximately room temperature.
wherein the compound of formula VI is prepared comprising the step d of reacting a compound of formula VII

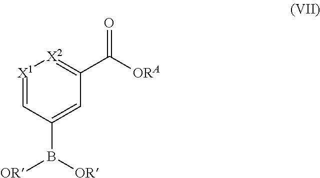

wherein the substituents are as defined above, R^{A} is a selected from C₁-C₇-alkyl and —B(OR′)₂ represents a cyclic or acyclic boronic acid or boronic acid derivative, such as pinaccolato-boron, with 6-bromo-4-chloro-quinazoline [38267-96-8] in the presence of a catalyst, such as a Pd(0) catalyst, e.g. Dichlorodiphenylphosphine palladium (PdCl₂(PPh₃)₂), optionally in the presence of one or more reaction aids, such as a base, e.g. an aqueous base, optionally in the presence of one or more diluents, particularly polar solvents, e.g. acetonitrile. The reaction is stirred at a temperature of approximately 100-120° C. e.g. in a microwaves oven. The reaction may be carried out under an inert gas such as nitrogen or argon. This type of reaction is also known as Suzuki reaction, typical reaction conditions are known in the field and may applied to the present process.

In a further embodiment, the invention relates to a process for manufacturing a compound of formula I (Method B) comprising the step e of reacting a compound of formula VIII

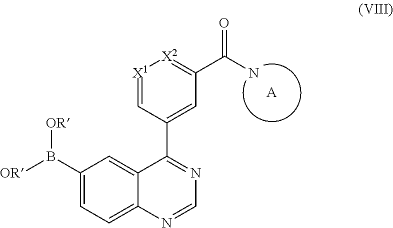

wherein the substituents are as defined above and —B(OR′)₂ represents a cyclic or acyclic boronic acid or boronic acid derivative, such as pinaccolato-boron, with a compound of formula IX,

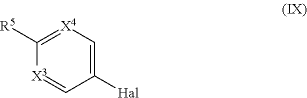

wherein the substituents are as defined above and Hal represents halogen, particularly iodo or bromo, in the presence of a catalyst, such as a Pd(0) catalyst, e.g. Pd(PPh₃)₄, optionally in the presence of one or more reaction aids, such as a base e.g. an aqueous base, optionally in the presence of one or more diluents, particularly polar solvents, e.g. acetonitrile. The reaction is stirred at a temperature of approximately 100-120° C. e.g. in a microwaves oven. The reaction may be carried out under an inert gas such as nitrogen or argon. This type of reaction is also known as Suzuki reaction, typical reaction conditions are known in the field and may applied to the present process;
wherein the compound of formula VIII is prepared comprising the step f of reacting a compound of formula II with a diboron derivative e.g. Bis-(pinacolato)-diboron in the presence of a palladium catalyst e.g. 1,1-Bis(diphenylphosphino)ferrocene]dichloropalladium (PdCl₂(dppf)-CH₂Cl₂), optionally in the presence of one or more reaction aids, such as a base e.g. an aqueous base such as potassium acetate, optionally in the presence of one or more diluents, particularly polar solvents, e.g. dioxane. The reaction is stirred at approximately 80° C. for several hours;
wherein the compound of formula II is prepared comprising the steps b, c and d of Method A.

In a further embodiment, the invention relates to a process for manufacturing a compound of formula I (Method C), comprising the step g of reacting a compound of formula X

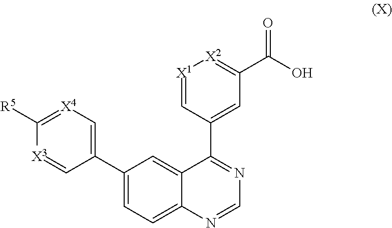

wherein the substituents are as defined above, with an amine of formula V under customary condensation conditions. The reaction is carried on by dissolving the carboxylic acid and the amine of formula V in a suitable solvent, for example halogenated hydrocarbon, such as methylene chloride, N,N-dimethylformamide, N,N-dimethylacetamide, N-2-methyl-pyrrolidone, methylene chloride, or a mixture of two or more such solvents, and by the addition of a suitable base, for example triethylamine, diisopropylethylamine (DIPEA) or N-methylmorpholine and a suitable coupling agent that forms a reactive derivative of the carboxylic acid in situ, for example (2-(1H-Benzotriazole-1-yl)-1,1,3,3-tetramethyluronium hexafluorophosphate (HBTU). The reaction mixture is stirred at a temperature of from approximately −20 to 50° C., such as from −5° C. to 30° C., e.g. at 0° C. to room temperature. The reaction may be carried out under an inert gas, e.g. nitrogen or argon;
wherein the compound of formula X is prepared comprising the step h of saponifying a compound of formula XI

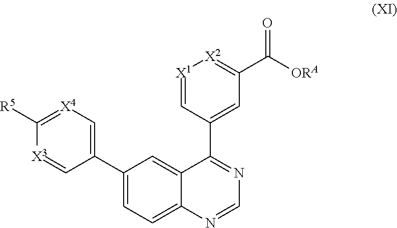

wherein the substituents are as defined above and R^{A} is a selected from C₁-C₇-alkyl. Saponification of the carboxylic ester is performed under customary saponification conditions, in the presence of an aqueous bases such as for example lithium hydroxide and organic solvent such as for example dioxane. The reaction is carried out at approximately room temperature;
wherein the compound of formula XI is prepared comprising the step i of reacting a compound of formula VI with a compound of formula III in the presence of a catalyst, such as a Pd(0) catalyst, e.g. Pd(PPh₃)₄, optionally in the presence of one or more reaction aids, such as a base e.g. an aqueous base, optionally in the presence of one or more diluents, particularly polar solvents, e.g. acetonitrile. The reaction is stirred at a temperature of approximately 100-120° C. e.g. in a microwaves oven. The reaction may be carried out under an inert gas such as nitrogen or argon. This type of reaction is also known as Suzuki reaction, typical reaction conditions are known in the field and may applied to the present process;
wherein the compound of formula VI is prepared comprising the step d of Method A.

In a further embodiment, the invention relates to a process for manufacturing a compound of formula I (Method D), comprising the step a of reacting a compound of formula II with a compound of formula III;

wherein the compound of formula II is prepared comprising the step j of reacting a compound of formula XI

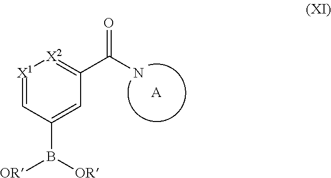

wherein the substituents are as defined above and —B(OR′)₂ represents a cyclic or acyclic boronic acid or boronic acid derivative, such as pinaccolato-boron, with 6-bromo-4-chloro-quinazoline [38267-96-8] in the presence of a catalyst, such as a Pd(0) catalyst, e.g. Dichlorodiphenylphosphine palladium (PdCl₂(PPh₃)₂), optionally in the presence of one or more reaction aids, such as a base, e.g. an aqueous base, optionally in the presence of one or more diluents, particularly polar solvents, e.g. acetonitrile. The reaction is stirred at a temperature of approximately 100-120° C. e.g. in a microwaves oven. The reaction may be carried out under an inert gas such as nitrogen or argon. This type of reaction is also known as Suzuki reaction, typical reaction conditions are known in the field and may applied to the present process;
wherein the compound of formula XI is prepared comprising the step k of reacting a compound of formula XII

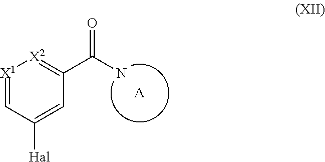

wherein the substituents are as defined above and Hal represents halogen, particularly iodo or bromo, with a diboron derivative e.g. Bis-(pinacolato)-diboron in the presence of a palladium catalyst e.g. 1,1-Bis(diphenylphosphino)ferrocene]dichloropalladium (PdCl₂(dppf)-CH₂Cl₂), optionally in the presence of one or more reaction aids, such as a base e.g. an aqueous base such as potassium acetate, optionally in the presence of one or more diluents, e.g. polar solvents, e.g. dioxane. The reaction is stirred at approximately 80° C. for several hours;
wherein the compound of formula XII is prepared comprising the step I of reacting a compound of formula XIII

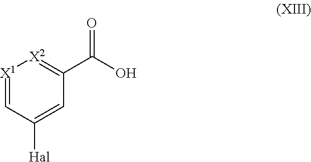

wherein the substituents are as defined above and Hal represents halogen, particularly iodo or bromo, with an amine of formula V under customary condensation conditions. The reaction is carried on by dissolving the carboxylic acid and the amine of formula V in a suitable solvent, for example halogenated hydrocarbon, such as methylene chloride, N,N-dimethylformamide, N,N-dimethylacetamide, N-2-methyl-pyrrolidone, methylene chloride, or a mixture of two or more such solvents, and by the addition of a suitable base, for example triethylamine, diisopropylethylamine (DIPEA) or N-methylmorpholine and a suitable coupling agent that forms a reactive derivative of the carboxylic acid in situ, for example (2-(1H-Benzotriazole-1-yl)-1,1,3,3-tetramethyluronium hexafluorophosphate (HBTU). The reaction mixture is stirred at a temperature of from approximately −20 to 50° C., e.g. from −5° C. to 30° C., e.g. at 0° C. to room temperature. The reaction may be carried out under an inert gas, e.g. nitrogen or argon.

In a further embodiment, the invention relates to a process for manufacturing a compound of formula I (Method E), comprising the step g of reacting a compound of formula X with an amine of formula V;

wherein the compound of formula X is prepared comprising the step m of reacting a compound of formula IV with a compound of formula III in the presence of a catalyst, such as a Pd(0) catalyst, e.g. Pd(PPh₃)₄, optionally in the presence of one or more reaction aids, such as a base e.g. an aqueous base, optionally in the presence of one or more diluents, particularly polar solvents, e.g. acetonitrile. The reaction is stirred at a temperature of approximately 100-120° C. e.g. in a microwaves oven. The reaction may be carried out under an inert gas such as nitrogen or argon. This type of reaction is also known as Suzuki reaction, typical reaction conditions are known in the field and may applied to the present process;
wherein the compound of formula IV is prepared comprising the step n of reacting a compound of formula XIV

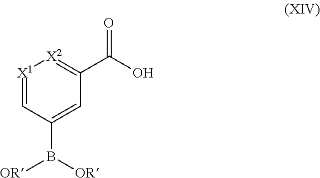

wherein the substituents are as defined above and —B(OR′)₂ represents a cyclic or acyclic boronic acid or boronic acid derivative, such as pinaccolato-boron, with 6-bromo-4-chloro-quinazoline [38267-96-8] in the presence of a catalyst, such as a Pd(0) catalyst, e.g. Dichlorodiphenylphosphine palladium (PdCl₂(PPh₃)₂), optionally in the presence of one or more reaction aids, such as a base, e.g. an aqueous base, optionally in the presence of one or more diluents, particularly polar solvents, e.g. acetonitrile. The reaction is stirred at a temperature of approximately 100-120° C. e.g. in a microwaves oven. The reaction may be carried out under an inert gas such as nitrogen or argon. This type of reaction is also known as Suzuki reaction, typical reaction conditions are known in the field and may applied to the present process;
wherein the compound of formula XIV is prepared comprising the step o of reacting a compound of formula XV

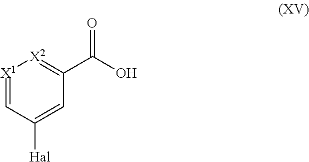

wherein the substituents are as defined above and Hal represents halogen, particularly iodo or bromo, with a diboron derivative e.g. Bis-(pinacolato)-diboron in the presence of a palladium catalyst e.g. 1,1-Bis(diphenylphosphino)ferrocene]dichloropalladium (PdCl₂(dppf)-CH₂Cl₂), optionally in the presence of one or more reaction aids, such as a base e.g. an aqueous base such as potassium acetate, optionally in the presence of one or more diluents, e.g. polar solvents, e.g. dioxane. The reaction is stirred at approximately 80° C. for several hours.

Protecting Groups:

In the methods describe above, functional groups which are present in the starting materials and are not intended to take part in the reaction, are present in protected form if necessary, and protecting groups that are present are cleaved, whereby said starting compounds may also exist in the form of salts provided that a salt-forming group is present and a reaction in salt form is possible. In additional process steps, carried out as desired, functional groups of the starting compounds which should not take part in the reaction may be present in unprotected form or may be protected for example by one or more protecting groups. The protecting groups are then wholly or partly removed according to one of the known methods. Protecting groups, and the manner in which they are introduced and removed are described, for example, in “Protective Groups in Organic Chemistry”, Plenum Press, London, New York 1973, and in “Methoden der organischen Chemie”, Houben-Weyl, 4th edition, Vol. 15/1, Georg-Thieme-Verlag, Stuttgart 1974 and in Theodora W. Greene, “Protective Groups in Organic Synthesis”, John Wiley & Sons, New York 1981. A characteristic of protecting groups is that they can be removed readily, i.e. without the occurrence of undesired secondary reactions, for example by solvolysis, reduction, photolysis or alternatively under physiological conditions.

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure antipodes.

Compounds of the invention and intermediates can also be converted into each other according to methods generally known to those skilled in the art.

Intermediates and final products can be worked up and/or purified according to standard methods, e.g. using chromatographic methods, distribution methods, (re-) crystallization, and the like.

The following applies in general to all processes mentioned herein before and hereinafter. All the above-mentioned process steps can be carried out under reaction conditions that are known to those skilled in the art, including those mentioned specifically, in the absence or, customarily, in the presence of solvents or diluents, including, for example, solvents or diluents that are inert towards the reagents used and dissolve them, in the absence or presence of catalysts, condensation or neutralizing agents, for example ion exchangers, such as cation exchangers, e.g. in the H+ form, depending on the nature of the reaction and/or of the reactants at reduced, normal or elevated temperature, for example in a temperature range of from about −100° C. to about 190° C., including, for example, from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers, for example diastereoisomers or enantiomers, or into any desired mixtures of isomers, for example racemates or mixtures of diastereoisomers, for example analogously to the methods described herein above.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofuran or dioxane, liquid aromatic hydrocarbons, such as benzene or toluene, alcohols, such as methanol, ethanol or 1- or 2-propanol, nitriles, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, methycyclohexane, or mixtures of those solvents, for example aqueous solutions, unless otherwise indicated in the description of the processes. Such solvent mixtures may also be used in working up, for example by chromatography or partitioning. The compounds, including their salts, may also be obtained in the form of hydrates, or their crystals may, for example, include the solvent used for crystallization. Different crystalline forms may be present.

The invention relates also to those forms of the process in which a compound obtainable as an intermediate at any stage of the process is used as starting material and the remaining process steps are carried out, or in which a starting material is formed under the reaction conditions or is used in the form of a derivative, for example in a protected form or in the form of a salt, or a compound obtainable by the process according to the invention is produced under the process conditions and processed further in situ.

All starting materials, building blocks, reagents, acids, bases, dehydrating agents, solvents and catalysts utilized to synthesize the compounds of the present invention are either commercially available or can be produced by organic synthesis methods known to one of ordinary skill in the art (Houben-Weyl 4^{th} Ed. 1952, Methods of Organic Synthesis, Thieme, Volume 21).

Members of the phosphoinositide-3 kinase (PI3K) family are involved in cell growth, differentiation, survival, cytoskeletal remodeling and the trafficking of intracellular organelles in many different types of cells (Okkenhaug and Wymann, Nature Rev. Immunol. 3:317 (2003).

To date, eight mammalian PI3Ks have been identified, divided into three main classes (I, II and III) on the basis of their genetic sequence, structure, adapter molecules, expression, mode of activation, and preferred substrate.

PI3Kδ is a lipid kinase belonging to the class I PI3K family (PI3K α, β, γ and 6) that generates second messenger signals downstream of tyrosine kinase-linked receptors.

PI3Kδ is a heterodimer composed of an adaptor protein and a p110δ catalytic subunit which converts phosphatidylinositol-4,5-bis-phosphate (PtdInsP2) to phosphatidylinositol-3,4,5-tri-phosphate (PtdInsP3). Effector proteins interact with PtdInsP3 and trigger specific signaling pathways involved in cell activation, differentiation, migration, and cell survival.

Expression of the p110δ and p110γ catalytic subunits is preferential to leukocytes. Expression is also observed in smooth muscle cells, myocytes and endothelial cells. In contrast, p110α and p110β are expressed by all cell types (Marone et al. Biochimica et Biophysica Acta 1784:159 (2008)).

PI3Kδ is associated with B cell development and function (Okkenhaug et al. Science 297:1031 (2002)).

B cells play also a critical role in the pathogenesis of a number of autoimmune and allergic diseases as well as in the process of transplant rejection (Martin and Chan, Annu. Rev. Immunol. 24:467 (2006)).

Chemotaxis is involved in many autoimmune or inflammatory diseases, in angiogenesis, invasion/metastasis, neurodegeneration or wound healing (Gerard et al. Nat. Immunol. 2:108 (2001)). Temporarily distinct events in leukocyte migration in response to chemokines are fully dependent on PI3Kδ and PI3Kγ (Liu et al. Blood 110:1191 (2007)).

PI3Kα and PI3Kδ play an essential role in maintaining homeostasis and pharmacological inhibition of these molecular targets has been associated with cancer therapy (Maira et al. Expert Opin. Ther. Targets 12:223 (2008)).

PI3Kα is involved in insulin signaling and cellular growth pathways (Foukas et al. Nature 441:366 (2006)). PI3Kδ isoform-selective inhibition are expected to avoid potential side effects such as hyperglycemia, and metabolic or growth disregulation.

The invention relates in a third aspect to the use of compounds of the present invention as pharmaceuticals. Particularly, the compounds of formula I have valuable pharmacological properties, as described hereinbefore and hereinafter. The invention thus provides:
a compound of the formula (I) as defined herein, as pharmaceutical/for use as pharmaceutical;
a compound of the formula (I) as defined herein, as medicament/for use as medicament;
a compound of the formula (I) as defined herein, for the prevention and/or treatment of conditions, diseases or disorders which are mediated by the activity of the PI3K enzymes, preferably by the activity of the PI3Kδ;
the use of a compound of formula (I) as defined herein, for the manufacture of a medicament for the prevention and/or treatment of conditions, diseases or disorders which are mediated by the activity of the PI3K enzymes, preferably by the activity of the PI3Kδ;
the use of a compound of formula (I) as defined herein, for the prevention and/or treatment of conditions, diseases or disorders which are mediated by the activity of the PI3K enzymes, preferably by the activity of the PI3Kδ;
the use of a compound of formula I as defined herein for the inhibition of the PI3K, enzymes, preferably of PI3Kδ;
the use of a compound of formula (I) as defined herein, for the treatment of a disorder or disease selected from autoimmune disorders, inflammatory diseases, allergic diseases, airway diseases, such as asthma and COPD, transplant rejection, cancers eg of hematopoietic origin or solid tumors;
a method of modulating the activity of the PI3K enzymes, preferably PI3Kδ, in a subject, comprising the step of administering to a subject a therapeutically effective amount of a compound of formula I as defined herein;
a method for the treatment of a disorder or disease mediated by the PI3K enzymes, preferably by PI3Kδ. comprising the step of administering to a subject a therapeutically effective amount of a compound of formula (I) as defined herein;
a method for inhibition of the PI3K enzymes, preferably PI3Kδ, in a cell, comprising contacting said cell with an effective amount of a compound of formula I as defined herein.

As used herein, the term “subject” refers to an animal. Typically the animal is a mammal. A subject also refers to for example, primates (e.g., humans), cows, sheep, goats, horses, dogs, cats, rabbits, rats, mice, fish, birds and the like. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, a subject is “in need of” a treatment if such subject would benefit biologically, medically or in quality of life from such treatment.

The term “administration” or “administering” of the subject compound means providing a compound of the invention and prodrugs thereof to a subject in need of treatment. Administration “in combination with” one or more further therapeutic agents includes simultaneous (concurrent) and consecutive administration in any order, and in any route of administration.

The invention relates to the use of new quinazoline derivates for the prevention and/or treatment of conditions, diseases or disorders which are mediated by the activity of the PI3K enzymes.

The invention includes methods of treating conditions, diseases or disorders in which one or more of the inflammatory functions of B cells such as antibody production, antigen presentation, cytokine production or lymphoid organogenesis are abnormal or are undesirable including rheumatoid arthritis, pemphigus vulgaris, Idiopathic thrombocytopenia purpura, systemic lupus erythematodus, multiple sclerosis, myasthenia gravis, Sjögren's syndrome, autoimmune hemolytic anemia, ANCA-associated vasculitides, cryoglobulinemia, thrombotic thrombocytopenic purpura, chronic autoimmune urticaria, allergy (atopic dermatitis, contact dermatitis, allergic rhinitis), goodpasture's syndrome, and cancers of haematopoietic origin.

The invention includes methods of treating conditions, diseases or disorders in which one or more of the inflammatory functions of neutrophils, such as superoxide release, stimulated exocytosis, or chemoatractic migration are abnormal or are undesirable including rheumatoid arthritis, sepsis, pulmonary or respiratory disorders such as asthma, inflammatory dermatoses such as psoriasis and others.

The invention includes methods of treating conditions, diseases or disorders in which one or more of the inflammatory functions of basophil and mast cells such as chemoatractic migration or allergen-IgE-mediated degranulation are abnormal or are undesirable including allergic diseases (atopic dermatitis, contact dermatitis, allergic rhinitis) as well as other disorders such as COPD, asthma or emphysema.

The invention includes methods of treating conditions, diseases or disorders in which one or more of the inflammatory functions of T cells such as cytokine production or cell-mediated cytotoxicity abnormal or are undesirable including rheumatoid arthritis, multiple sclerosis, acute or chronic rejection of cell tissue or organ grafts or cancers of haematopoietic origin.

Further, the invention includes methods of treating neurodegenerative diseases, cardiovascular diseases and platelet aggregation.

In a further embodiment, the invention relates to a process or a method for the treatment of one of the disorders or diseases mentioned hereinabove, especially a disease which responds to the inhibition of the PI3K enzymes. The compounds of formula I, or a pharmaceutically acceptable salt thereof, can be administered as such or in the form of pharmaceutical compositions, prophylactically or therapeutically, preferably in an amount effective against the said diseases, to a warm-blooded animal, for example a human, requiring such treatment, the compounds especially being used in the form of pharmaceutical compositions.

In a further embodiment, the invention relates to the use of a compound of formula I, or a pharmaceutically acceptable salt thereof, as such or in the form of a pharmaceutical composition with at least one pharmaceutically acceptable carrier, for the therapeutic and also prophylactic management of one or more of the diseases mentioned hereinabove, mediated by the PI3K enzymes.

In a further embodiment, the invention relates to the use of a compound of formula I, or a pharmaceutically acceptable salt thereof, especially a compound of formula I which is said to be preferred, or a pharmaceutically acceptable salt thereof, for the preparation of a pharmaceutical composition for the therapeutic and also prophylactic management of one or more of the diseases mentioned hereinabove, especially a disorder or disease selected from autoimmune disorders, inflammatory diseases, allergic diseases, airway diseases, such as asthma and COPD, transplant rejection, cancers eg of hematopoietic origin or solid tumors.

The invention relates in a fourth aspect to pharmaceutical compositions comprising a compound of the present invention. The invention thus provides
a pharmaceutical composition comprising (i.e. containing or consisting of) a compound as defined herein and one or more carriers/excipients;
a pharmaceutical composition comprising a therapeutically effective amount of a compound of formula I as defined herein, and one or more pharmaceutically acceptable carriers/excipients.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The present invention provides a pharmaceutical composition comprising a compound of the present invention and a pharmaceutically acceptable carrier. The pharmaceutical composition can be formulated for particular routes of administration such as oral administration, parenteral administration, and rectal administration, etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art. Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

The present invention further provides anhydrous pharmaceutical compositions and dosage forms comprising the compounds of the present invention as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

Suitable excipients/carriers may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like.

Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of the formula (I) in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The dosage of the active ingredient depends upon the disease to be treated and upon the species, its age, weight, and individual condition, the individual pharmacokinetic data, and the mode of administration. The amount of the compound in a formulation can vary within the full range employed by those skilled in the art. Typically, the formulation will contain, on a weight percent (wt %) basis, from about 0.01-99.99 wt % of a compound of formula (I) based on the total formulation, with the balance being one or more suitable pharmaceutical excipients.

Pharmaceutical compositions comprising a compound of formula (I) as defined herein in association with at least one pharmaceutical acceptable carrier (such as excipient a and/or diluent) may be manufactured in conventional manner, e.g. by means of conventional mixing, granulating, coating, dissolving or lyophilising processes.

In a further embodiment, the invention relates to a pharmaceutical composition for administration to a warm-blooded animal, especially humans or commercially useful mammals suffering from a disease which responds to an inhibition of the PI3K enzymes, comprising an effective quantity of a compound of formula I for the inhibition of the PI3K enzymes, or a pharmaceutically acceptable salt thereof, together with at least one pharmaceutically acceptable carrier.

In a further embodiment, the invention relates to a pharmaceutical composition for the prophylactic or especially therapeutic management of a disorder or disease selected from autoimmune disorders, inflammatory diseases, allergic diseases, airway diseases, such as asthma and COPD, transplant rejection, cancers eg of hematopoietic origin or solid tumors; of a warm-blooded animal, especially a human or a commercially useful mammal requiring such treatment.

The invention relates in a fifth aspect to combinations comprising a compound of formula I and one or more additional active ingredients. The invention thus provides
a combination in particular a pharmaceutical combination, comprising a therapeutically effective amount of a compound of formula I and one or more therapeutically active agents, e.g. an immunosuppressant, immunomodulatory, anti-inflammatory or chemotherapeutic agent, e.g. as indicated below;
a combined pharmaceutical composition, adapted for simultaneous or sequential administration, comprising a therapeutically effective amount of a compound of formula (I) as defined herein; therapeutically effective amount(s) of one or more combination partners e.g. an immunosuppressant, immunomodulatory, anti-inflammatory or chemotherapeutic agent, e.g. as indicated below; one or more pharmaceutically acceptable excepients;
a combined pharmaceutical composition as defined herein (i) as pharmaceutical, (ii) for use in the treatment of a disease mediated by the PI3K enzymes, (iii) in a method of treatment of a disease mediated by the PI3K enzymes.

By “combination”, there is meant either a fixed combination in one dosage unit form, or a kit of parts for the combined administration where a compound of the formula (I) and a combination partner may be administered independently at the same time or separately within time intervals that especially allow that the combination partners show a cooperative, e.g. synergistic effect.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviating, inhibiting, preventing and/or ameliorating a condition, or a disorder or a disease (i) mediated by the dysregulation of PI3K delta, or (ii) associated with the dysregulation of PI3K delta, or (iii) characterized by the dysregulation of the PI3K delta; or (2) reducing or inhibiting the activity of the PI3K delta. In another non-limiting embodiment, the term “a therapeutically effective amount” refers to the amount of the compound of the present invention that, when administered to a cell, or a tissue, or a non-cellular biological material, or a medium, is effective to at least partially reducing or inhibiting PI3K delta.

The compounds of formula I may be administered as the sole active ingredient or in conjunction with, e.g. as an adjuvant to, other drugs e.g. immunosuppressive or immunomodulating agents or other anti-inflammatory agents, e.g. for the treatment or prevention of allo- or xenograft acute or chronic rejection or inflammatory or autoimmune disorders, or a chemotherapeutic agent, e.g a malignant cell anti-proliferative agent. For example, the compounds of formula I may be used in combination with a calcineurin inhibitor, e.g. cyclosporin A or FK 506; a mTOR inhibitor, e.g. rapamycin, 40-O-(2-hydroxyethyl)-rapamycin, CCI779, ABT578, AP23573, biolimus-7 or biolimus-9; an ascomycin having immuno-suppressive properties, e.g. ABT-281, ASM981, etc.; corticosteroids; cyclophosphamide; azathioprene; methotrexate; leflunomide; mizoribine; mycophenolic acid or salt; mycophenolate mofetil; 15-deoxyspergualine or an immunosuppressive homologue, analogue or derivative thereof; a PKC inhibitor, e.g. as disclosed in WO 02/38561 or WO 03/82859, e.g. the compound of Example 56 or 70; a JAK3 kinase inhibitor, e.g. N-benzyl-3,4-dihydroxy-benzylidene-cyanoacetamide α-cyano-(3,4-dihydroxy)-]N-benzylcinnamamide (Tyrphostin AG 490), prodigiosin 25-C(PNU156804), [4-(4′-hydroxyphenyl)-amino-6,7-dimethoxyquinazoline] (WHI-P131), [4-(3′-bromo-4′-hydroxylphenyl)-amino-6,7-dimethoxyquinazoline] (WHI-P154), [4-(3′,5′-dibromo-4′-hydroxylphenyl)-amino-6,7-dimethoxyquinazoline] WHI-P97, KRX-211, 3-{(3R,4R)-4-methyl-3-[methyl-(7H-pyrrolo[2,3-d]pyrimidin-4-yl)-amino]-piperidin-1-yl}-3-oxo-propionitrile, in free form or in a pharmaceutically acceptable salt form, e.g. mono-citrate (also called CP-690,550), or a compound as disclosed in WO 04/052359 or WO 05/066156; immunosuppressive monoclonal antibodies, e.g., monoclonal antibodies to leukocyte receptors, e.g., MHC, CD2, CD3, CD4, CD7, CD8, CD25, CD28, CD40, CD45, CD52, CD58, CD80, CD86 or their ligands; other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a mutant thereof, e.g. LEA29Y; adhesion molecule inhibitors, e.g. LFA-1 antagonists, ICAM-1 or -3 antagonists, VCAM-4 antagonists or VLA-4 antagonists; or a chemotherapeutic agent, e.g. paclitaxel, gemcitabine, cisplatinum, doxorubicin or 5-fluorouracil; or antihistamines; or antitussives, or a bronchodilatory agent; or an angiotensin receptor blockers; or an anti-infectious agent.

Where the compounds of formula I are administered in conjunction with other immunosuppressive/immunomodulatory, anti-inflammatory, chemotherapeutic or anti-infectious therapy, dosages of the co-administered immunosuppressant, immunomodulatory, anti-inflammatory, chemotherapeutic or anti-infectious compound will of course vary depending on the type of co-drug employed, e.g. whether it is a steroid or a calcineurin inhibitor, on the specific drug employed, on the condition being treated and so forth.

### EXPERIMENTAL DETAILS

Insofar as the production of the starting materials is not particularly described, the compounds are known or may be prepared analogously to methods known in the art or as described hereafter.

The following examples are illustrative of the invention without any limitation.

### ABBREVIATIONS

Ar aryl
BOC tert-Butyl-carbonate
br.s. broad singlet
CH₂Cl₂ Dichloromethane
CH₃CN Acetonitril
d doublet
dd doublet of doublets
DIPEA N-ethyldiisopropylamine
DME 1,4-dimethoxyethane
DMF N,N-dimethylformamide
DMSO dimethylsulfoxide
dt doublet of triplets
EtOAc Ethyl acetate
FCC flash column chromatography
h hour
HBTU (2-(1H-Benzotriazole-1-yl)-1,1,3,3-tetramethyluronium hexafluorophosphate
HPLC high pressure liquid chromatography
HT high throughput
H₂O water
K kelvin
LC liquid chromatography
M molar
MeCN acetonitril
MeOD methanol-d4
MeOH methanol
MgSO4 magnesium sulfate
MHz mega hertz
MS mass spectroscopy
m multiplet
min. minute
mw microwave
Na₂SO₄ sodium sulfate
NEt₃ triethylamine
NH₃ ammonia
NMR nuclear magnetic resonance
PL-HCO₃ MP polymer supported hydrogen carbonate macroporous polystyrene
PL-SO₃H MP polymer supported sulfonic acid macroporous polystyrene
rt room temperature
Rt Retention time
s singulet
SCx-2 polymer supported sulfonic acid macroporous polystyrene
t triplet
TFA trifluoroacetic acid
THF tetrahydrofuran
UPLC ultra performance liquid chromatography

All compounds are named using AutoNom.

LC Specificity:

LC methode 1 (Rt⁽¹⁾):

The retention times (Rt) were obtained on a Waters HPLC alliance-HT system with a XBridge MS column C18 30/3.0 2.5 m applying a gradient H₂O (+0.1% formic acid)/CH₃CN (+0.1% formic acid) 90/10 to 5/95 over 1.7 minutes and 1.2 mL/min. as solvent flow and 40° C. for the oven temperature.

LC methode 2 (Rt⁽²⁾):

The retention times (Rt) were obtained on a Waters HPLC alliance-HT system with a XBridge MS column C18 30/3.0 2.5 m applying a gradient H₂O (+0.1% TFA)/CH₃CN (+0.1% TFA) 90/10 to 5/95 over 1.7 minutes and 1.2 mL/min. as solvent flow and 40° C. for the oven temperature.

LC methode 3 (Rt⁽³⁾):

The retention times (Rt) were obtained on a Waters HPLC alliance-HT system with a XBridge MS column C18 30/3.0 2.5 m applying a gradient H₂O (+0.1% TFA)/CH₃CN (+0.1% TFA) 95/5 to 5/95 over 3.7 minutes and 1.2 mL/min. as solvent flow and 40° C. for the oven temperature.

LC methode 4 (Rt⁽⁴⁾):

The retention times (Rt) were obtained on a Waters HPLC alliance-HT system with a SunFire column C18 20×4.6 mmm applying a gradient H₂O (+0.1% TFA)/CH₃CN (+0.1% TFA) 95/5 to 0/100 over 4 minutes and 1 mL/min. as solvent flow and 45° C. for the oven temperature.

LC methode 5 (Rt⁽⁵⁾):

The retention times (Rt) were obtained on a Waters UPLC-MS system with a Acquity UPLC BEH C18 50×2.1 mm, 1.7 um column applying a gradient H₂O (+0.1% formic acid)/CH₃CN (+0.1% formic acid) 95/5 to 10/90 over 4 minutes and 0.7 mL/min. as solvent flow and 30° C. for the oven temperature.

LC methode 6 (Rt⁽⁶⁾):

The retention times (Rt) were obtained on a Waters UPLC-MS system with a Acquity UPLC BEH C18 50×2.1 mm, 1.7 um column applying a gradient H₂O (+0.1% formic acid)/CH₃CN (+0.1% formic acid) 80/20 to 5/95 over 4.2 minutes and 0.7 mL/min. as solvent flow and 30° C. for the oven temperature.

LC methode 7 (Rt⁽⁷⁾):

The retention times (Rt) were obtained on a Waters HPLC alliance-HT system with a XBridge MS column C18 30/3.0 2.5 m applying a gradient H₂O (+0.1% formic acid)/CH₃CN (+0.1% formic acid) 95/5 to 5/95 over 3.7 minutes and 1.2 mL/min. as solvent flow and 40° C. for the oven temperature.

LC methode 8 (Rt⁽⁸⁾):

The retention times (Rt) were obtained on a Waters HPLC alliance-HT system with a XBridge MS column C18 30/3.0 2.5 m applying a gradient H₂O (+0.1% formic acid)/CH₃CN (+0.1% formic acid) 99/1 to 5/95 over 2.2 minutes and 1.2 mL/min. as solvent flow and 40° C. for the oven temperature.

LC methode 9 (Rt⁽⁹⁾):

The retention times (Rt) were obtained on a Waters HPLC alliance-HT system with a XBridge MS column C18 30/3.0 2.5 m applying a gradient H₂O (+0.1% TFA)/CH₃CN (+0.1% TFA) 99/1 to 5/95 over 2.2 minutes and 1.2 mL/min. as solvent flow and 40° C. for the oven temperature.

LC methode 10 (Rt⁽¹⁰⁾): The FIA-MS (MS) were obtained on a Waters HPLC-MS instrument.

### Preparation of intermediate compounds

### Intermediate 1: 5-Bromo-2-methoxy-3-trifluoromethyl-pyridine

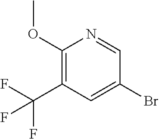

2-Methoxy-3-trifluoromethyl-pyridine (2.7 g, 14.79 mmol) and 1,3-dibromo-5,5-dimethylhydantoin (5.28 g, 18.48 mmol) were placed in a round-bottom flask. To this mixture was slowly added 40 ml TFA. The mixture was stirred overnight at ambient temperature (16 h). After completion of the reaction, TFA solvent was evaporated in vacuo and the resulting residue was neutralized to pH6-7 by the addition of saturated NaHCO₃. The aqueous layer was extracted with DCM two times and the combined extract was washed with brine, dried over magnesium sulfate and concentrated in vacuo to give a mixture of oil and white solid. The residue was redissolved into 20% Ethylacetate/Heptane (50 ml) and the insoluble white solid was filtered off. The filtrate was concentrated and then purified by Flash-chromatography on silica gel (EtOAc/Heptane 5/95) to give 5-Bromo-2-methoxy-3-trifluoromethyl-pyridine as a colorless liquid (2.08 g, 52% yield).

¹H-NMR (400 MHz, DMSO-d₆, 298 K): δ ppm 4.03 (s, 3H) 7.95 (d, 1H) 8.4 (d, 1H).

### 2-Methoxy-3-trifluoromethyl-pyridine

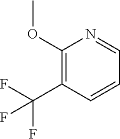

2-Chloro-3-trifluoromethyl-pyridine (3 g, 16.53 mmol) was dissolved in 30 ml of a solution of sodium methoxide (5.4M) in methanol. The mixture was stirred at ambient temperature for 2 days. After this period of time, the reaction was taken into ice and extracted with DCM three times. The combined extract was washed with brine, dried over magnesium sulfate and concentrated in vacuo to give 2-methoxy-3-trifluoromethyl-pyridine as a light liquid (2.7 g, 89% yield). ¹H-NMR (400 MHz, DMSO-d₆, 298 K): δ ppm 3.98 (s, 3H) 7.2 (dd, 1H) 8.11 (d, 1H) 8.45 (d, 1H). MS: 178.1 [M+1]⁺, Rt⁽¹⁾=1.29 min.

### Intermediate 2: 5-Bromo-2-ethoxy-3-trifluoromethyl-pyridine

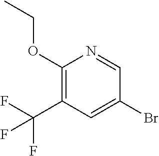

Intermediate 2 was prepared according the procedure described for intermediate 1 using a solution of sodium ethoxyde in ethanol instead of a solution of sodium methoxide. ¹H-NMR (400 MHz, DMSO-d₆, 298 K): δ ppm 1.33 (t, 4H) 4.45 (q, 3H) 8.31 (s, 1H) 8.58 (s, 1H).

### Intermediate 3: 1-[4-(5-Bromo-2-methyl-benzoyl)-piperazin-1-yl]-ethanone

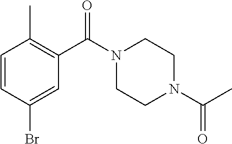 [SMILES: CC(=O)N1CCN(C(=O)c2cc(Br)ccc2C)CC1]

To a mixture of 5-bromo-2-methylbenzoic acid (2.0 g, 9.30 mmol) in DCM (25 mL) was added DIPEA (3.25 mL, 18.60 mmol) and HBTU (4.23 g, 11.16 mmol) at rt. The reaction mixture was stirred at rt for 20 min. To the mixture was then added 1-(piperazin-1-yl)ethanone (1.311 g, 10.23 mmol) and the reaction mixture was stirred at rt for 1 hour. The reaction was quenched with a saturated aqueous solution of NaHCO₃ and extracted with DCM. The organic layer was washed twice with brine, dried by passing through a phase separating cartridge and evaporated. Purification by Flash chromatography using Biotage Isolera system (amine functionalized silica KP-NH, eluting with Cyclohexane/EtOAc 0 to 100%) gave the title compound (2.475 g, 82% yield) as a white foam. MS: 325.4 [M+1]⁺, Rt⁽²⁾=0.94 min.

### Intermediate 4: 1-[4-(3-Bromo-5-trifluoromethyl-benzoyl)-piperazin-1-yl]-ethanone

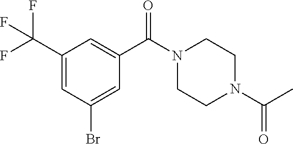 [SMILES: CC(=O)N1CCN(C(=O)c2cc(Br)cc(C(F)(F)F)c2)CC1]

Intermediate 4 was prepared according the procedure described for intermediate 3 using 3-bromo-5-trifluoromethylbenzoic acid instead of 5-bromo-2-methylbenzoic acid. MS: 379.3-381.3 [M+H]⁺, Rt⁽²⁾=1.129 min.

### Intermediate 5: 1-[4-(3-Bromo-5-methoxy-benzoyl)-piperazin-1-yl]-ethanone

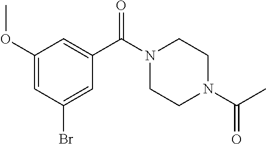 [SMILES: COc1cc(Br)cc(C(=O)N2CCN(C(C)=O)CC2)c1]

Intermediate 5 was prepared according the procedure described for intermediate 3 using 3-bromo-5-methoxybenzoic acid (intermediate 17) instead of 5-bromo-2-methylbenzoic acid. MS: 343.2 [M+H]⁺, Rt⁽²⁾=1.02 min.

### Intermediate 6: 1-[4-(3-Bromo-5-methyl-benzoyl)-piperazin-1-yl]-ethanone

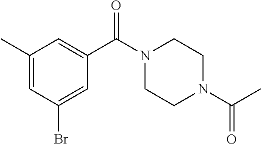 [SMILES: CC(=O)N1CCN(C(=O)c2cc(C)cc(Br)c2)CC1]

Intermediate 6 was prepared according the procedure described for intermediate 3 using 3-bromo-5-methoxybenzoic acid (intermediate 17) instead of 5-bromo-2-methylbenzoic acid. MS: 325.2-327.1 [M+H]⁺, Rt⁽²⁾=0.98 min.

### Intermediate 7: 1-[4-(3-Bromo-5-chloro-benzoyl)-piperazin-1-yl]-ethanone

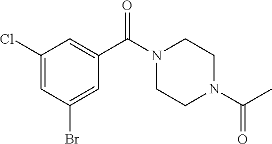 [SMILES: CC(=O)N1CCN(C(=O)c2cc(Cl)cc(Br)c2)CC1]

Intermediate 7 was prepared according the procedure described for intermediate 3 using 3-bromo-5-methoxybenzoic acid (intermediate 17) instead of 5-bromo-2-methylbenzoic acid. MS: 345.2-347.1-349.0 [M+H]⁺, Rt⁽²⁾=1.02 min.

### Intermediate 8: N-(4-bromo-2-(trifluoromethyl)phenyl)methanesulfonamide

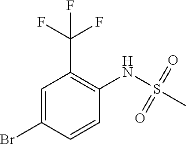 [SMILES: CS(=O)(=O)Nc1ccc(Br)cc1C(F)(F)F]

To a mixture of 2-amino-5-bromobenzotrifluoride (1.0 g, 4.17 mmol) in DCM (10 mL) at 0-5° C. was added NEt₃ (1.16 mL, 8.33 mmol), then methanesulfonyl chloride (0.389 mL, 5 mmol) dropwise. The reaction mixture was stirred at rt for 4 days. After 2 days, more NEt₃ was added (1.16 mL, 8.33 mmol). As there was no evolution after 3 days, more NEt₃ (0.580 mL, 4.17 mmol) and methanesulfonyl chloride (0.324 mL, 4.17 mmol) were added. The reaction was not completed, so the reaction mixture was then heated in a microwave oven at 110° C. for 20 min. There was no evolution, so the reaction was stopped. The reaction mixture was diluted with water and DCM. Layers were separated. The organic layer was washed with water, dried over MgSO₄ and evaporated. Purification by Flash chromatography using CombiFlash Companion ISCO system (Redisep silica 40 g column, eluting with Cyclohexane/EtOAc 100:0 to 70:30) did not give the pure compound. Purification by prep HPLC using Gilson system (SunFire C18 column, eluting with H₂O+0.1% TFA/CH₃CN 20% to 85%) gave the title compound (404 mg, 31% yield) as a white solid. ¹H-NMR (400 MHz, DMSO-d₆, 298 K): δ ppm 3.12 (s, 3H) 7.55 (d, 1H) 7.91 (d, 1H) 7.92 (s, 1H) 9.56 (s, 1H). MS⁽¹⁰⁾: 316.3-318.2 [M−1]⁻.

### Intermediate 9: N-(3-bromo-5-(trifluoromethyl)phenyl)methane sulfonamide

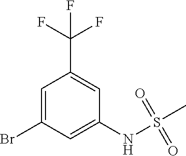 [SMILES: CS(=O)(=O)Nc1cc(Br)cc(C(F)(F)F)c1]

To a mixture of 3-amino-5-bromobenzotrifluoride (1.0 g, 4.17 mmol) in pyridine (10 mL) at 0-5° C. was added dropwise methanesulfonyl chloride (0.389 mL, 5 mmol). The reaction mixture was stirred at rt for 4 days. As the reaction was not completed, the reaction mixture was then heated in a microwave oven at 150° C. for 15 min. There was no evolution, so the reaction was stopped. The reaction mixture was concentrated until dryness, and the residue was co-evaporated with toluene. The residue was then diluted with a saturated aqueous solution of NaHCO₃ and extracted with DCM. The organic layer was dried over MgSO₄ and evaporated. Purification by Flash chromatography using CombiFlash Companion ISCO system (Redisep silica 12 g column, eluting with Cyclohexane/EtOAc 100:0 to 70:30) gave the title compound (1.05 g, 79% yield) as a white solid. ¹H-NMR (400 MHz, DMSO-d₆, 298 K): δ ppm 3.14 (s, 3H) 7.48 (s, 1H) 7.64 (s, 1H) 7.68 (s, 1H) 10.42 (s, 1H). MS⁽¹⁰⁾: 316.3-318.2 [M−1]⁻.

### Intermediate 10: 2-Difluoromethoxy-5-(4,4,5,5-tetramethyl-[1,3,2]dioxaborolan-2-yl)-pyridine

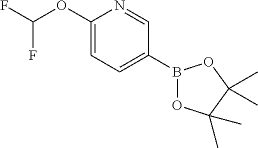 [SMILES: CC1(C)OB(c2ccc(OC(F)F)nc2)OC1(C)C]

In a sealed tube was added 2-hydroxy-5-(4,4,5,5-tetramethyl-1,3,2-dioxaborolan-2-yl)pyridine (300 mg, 1.357 mmol), sodium chlorodifluoroacetate (320 mg, 2.036 mmol) in acetonitrile (5 mL). This suspension was heated to 80° C. and stirred overnight. The reaction mixture was cooled down to rt, diluted with EtOAc, washed with an aqueous solution of NaHCO₃ and brine. The organic layer dried over MgSO₄, filtered and evaporated. Purification by flash chromatography on silica gel (CH2Cl2/MeOH, 95/5) gave the title compound (197 mg, 53% yield). MS: 272.8 [M+H]⁺, Rt⁽⁶⁾=3.12 min.

### Intermediate 11: 6,6-Difluoro-[1,4]diazepane

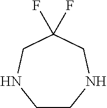

The compound was prepared following literature procedure: Wellner, E.; Sandin, H.; Synthesis; 2002; 2; 223-226. ¹H-NMR (400 MHz, DMSO-d₆, 298 K): δ ppm 3.47 (s, 4H) 3.89 (t, 4H)

The boronic acids or boronic esters described herein are prepared according the general procedure described below.

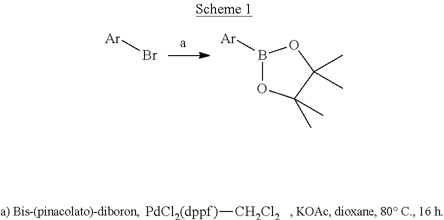

### Intermediate 12: 2-Methoxy-5-(4,4,5,5-tetramethyl-[1,3,2]dioxaborolan-2-yl)-nicotinonitrile

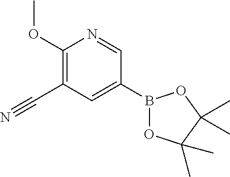 [SMILES: COc1ncc(B2OC(C)(C)C(C)(C)O2)cc1C#N]

Solution A: PdCl₂(dppf)-CH₂Cl₂ (0.958 g, 1.174 mmol), KOAc (6.91 g, 70.4 mmol) and Bis-(pinacolato)-diboron (7.15 g, 28.2 mmol) were placed into a 250 mL flask and degassed.

Solution B: In a separate vial, 5-bromo-2-methoxy nicotinitrile (5 g, 23.47 mmol) was dissolved in 100 mL of anhydrous dioxane. Solution B was added to solution A, and the reaction mixture heated to 80° C. for 16 h. The mixture was cooled down to rt, diluted with EtOAc and the remaining solid filtered off. The filtrate was evaporated under vacuum to yield a black oil. Purification by flash chromatography on silica gel (CH₂Cl₂/MeOH, 95/5) gave the title compound (5.7 g, 89% yield) as a beige powder. ¹H-NMR (400 MHz, DMSO-d₆, 298 K): δ ppm 1.31 (s, 12H) 4.03 (s, 3H) 8.31 (s, 1H) 8.62 (s, 1H). MS: 261.5 [M+1]⁺, Rt⁽²⁾=1.47 min.

Intermediate 13 to 22, were prepared using procedures analogous to those used for intermediate 12, using the corresponding Aryl bromide as starting materials.

| | | | MS(ES): |
|---|---|---|---|
| Intermediate | Structure | Rt (min.) | [M + H]⁺ |
| Intermediate 13 | 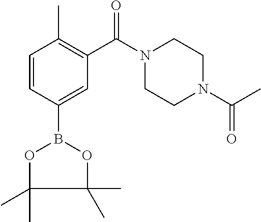 [SMILES: CC(=O)N1CCN(C(=O)c2cc(B3OC(C)(C)C(C)(C)O3)ccc2C)CC1] | 1.10 ⁽¹⁾ | 373.2 |
| Intermediate 14 | 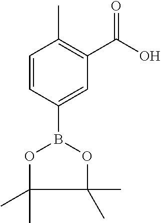 [SMILES: Cc1ccc(B2OC(C)(C)C(C)(C)O2)cc1C(=O)O] | 1.36 ⁽¹⁾ | 263.1 |
| Intermediate 15 | 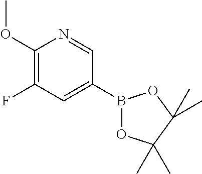 [SMILES: COc1ncc(B2OC(C)(C)C(C)(C)O2)cc1F] | 1.53 ⁽¹⁾ | 254.1 |
| Intermediate 16 | 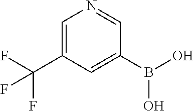 [SMILES: OB(O)c1cncc(C(F)(F)F)c1] | 0.64 ⁽¹⁾ | 191.9 |
| Intermediate 17 | 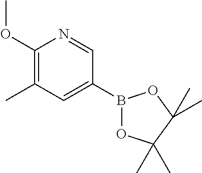 [SMILES: COc1ncc(B2OC(C)(C)C(C)(C)O2)cc1C] | 1.58 ⁽¹⁾ | 250.1 |
| Intermediate 18 | 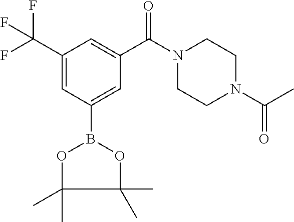 [SMILES: CC(=O)N1CCN(C(=O)c2cc(B3OC(C)(C)C(C)(C)O3)cc(C(F)(F)F)c2)CC1] | 1.29 ⁽¹⁾ | 427.3 |
| Intermediate 19 | 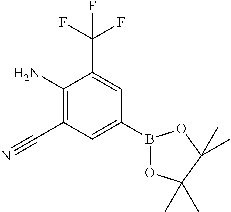 [SMILES: CC1(C)OB(c2cc(C#N)c(N)c(C(F)(F)F)c2)OC1(C)C] | 1.57 ⁽¹⁾ | No mass |
| Intermediate 20 | 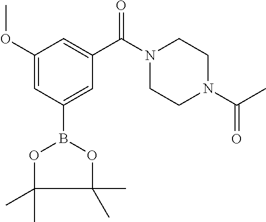 [SMILES: COc1cc(B2OC(C)(C)C(C)(C)O2)cc(C(=O)N2CCN(C(C)=O)CC2)c1] | 1.17 ⁽¹⁾ | 389.3 |
| Intermediate 21 | 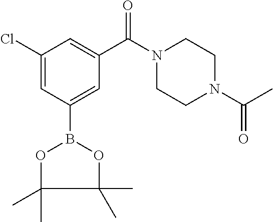 [SMILES: CC(=O)N1CCN(C(=O)c2cc(Cl)cc(B3OC(C)(C)C(C)(C)O3)c2)CC1] | 1.30 ⁽¹⁾ | 393.3 |
| Intermediate 22 | 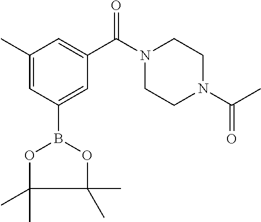 [SMILES: CC(=O)N1CCN(C(=O)c2cc(C)cc(B3OC(C)(C)C(C)(C)O3)c2)CC1] | 1.22 ⁽¹⁾ | 373.3 |

⁽¹⁾ LC methode 1,
⁽²⁾ LC methode 2

### Intermediate 23: 3-Bromo-5-methoxybenzoic acid

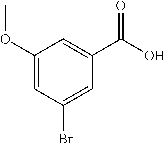

To a vigorously stirred mixture of 1-Bromo-3-methoxy-5-methylbenzene (1 g, 4.97 mmol), Pyridine (3.22 mL, 39.8 mmol) and Water (8 ml) was added in small portions KMnO₄ (3.14 g, 19.89 mmol) at 105° C. The mixture which turned to a black suspension was stirred 24 hours at 105° C., then cooled down to RT and filtered over Hyflo. The black residue was washed several times with EtOAc. The filtrate was then diluted in EtAOc and washed with a 2M solution of HCl. The organic layer was dried over sodium sulfate, filtered and concentrated to afford the title compound (281 mg, 24% yield) as a white solid. MS: 229.1 [M+H]⁺, Rt⁽¹⁾=1.18 min.

### Preparation of Final Compounds

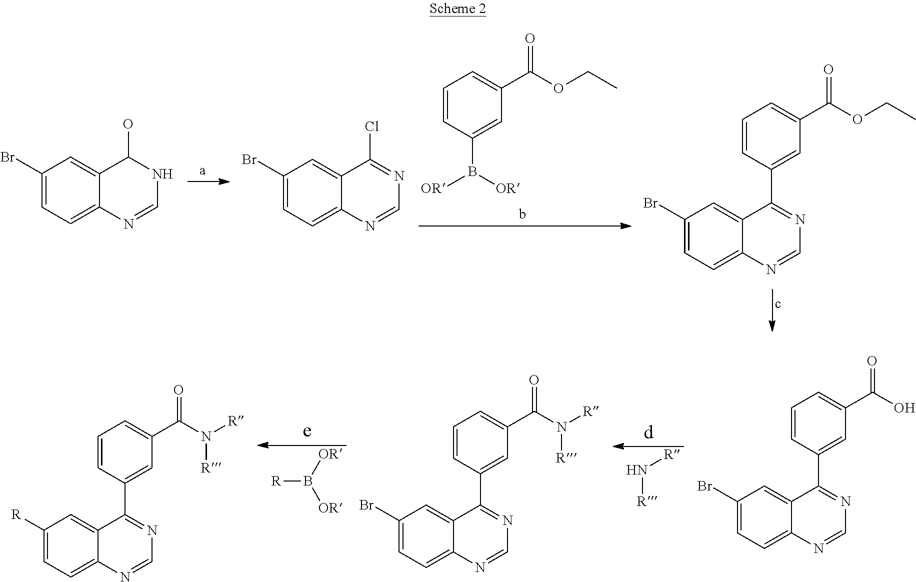

The final compounds described herein were according the general procedure described in scheme 2.

### Example 1

### 5-{4-[3-(4-Acetyl-piperazine-1-carbonyl)-phenyl]-quinazolin-6-yl}-2-methoxy-nicotinonitrile

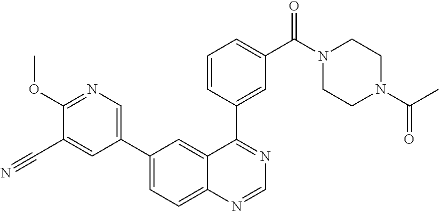 [SMILES: COc1ncc(-c2ccc3ncnc(-c4cccc(C(=O)N5CCN(C(C)=O)CC5)c4)c3c2)cc1C#N]

To a mixture of 1-{4-[3-(6-Bromo-quinazolin-4-yl)-benzoyl]-piperazin-1-yl}-ethanone (100 mg, 0.228 mmol), 2-Methoxy-5-(4,4,5,5-tetramethyl-[1,3,2]dioxaborolan-2-yl)-nicotinonitrile (89 mg, 0.273 mmol) and Pd(PPh₃)₄ (13.14 mg, 0.011 mmol) was added 3 mL of DME. The reaction mixture was flushed with argon and a 1M aqueous solution of Na₂CO₃ (0.455 mL, 0.455 mmol) was added and the vial was capped. The reaction mixture was heated to 140° C. for 10 min using a microwave oven then cooled down to rt, diluted with EtOAc, filtered through a Celite pad and portioned between H₂O/EtOAc. The organic layer was washed with brine, dried over MgSO₄, filtered and evaporated. Purification by preparative reverse phase Gilson HPLC and subsequent neutralization of the combined fractions over PL-HCO₃ MP gave the title compound (47 mg, 41% yield) as a white powder. ¹H-NMR (400 MHz, DMSO-d₆, 298 K): δ ppm 1.98 (br.s., 3H) 3.37-3.70 (m, 8H) 4.07 (s, 3H) 7.71 (dt, 1H) 7.75 (t, 1H) 7.91 (br.s., 1H) 8.04 (dt, 1H) 8.25 (d, 1H) 8.35 (br.s., 1H) 8.43 (dd, 1H) 8.80 (br.s., 1H) 8.92 (br.s., 1H) 9.41 (s, 1H). MS: 493.2 [M+1]⁺, Rt⁽¹⁾=1.14 min.

### 1-{4-[3-(6-Bromo-quinazolin-4-yl)-benzoyl]-piperazin-1-yl}-ethanone

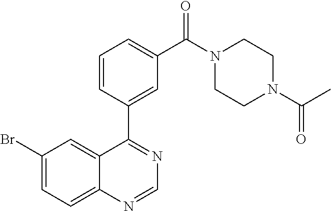 [SMILES: CC(=O)N1CCN(C(=O)c2cccc(-c3ncnc4ccc(Br)cc34)c2)CC1]

To a solution of 3-(6-Bromo-quinazolin-4-yl)-benzoic acid (2 g, 6.08 mmol) in 60 mL of CH₂Cl₂ was added HBTU (2.53 g, 6.68 mmol) and DIPEA (2.122 mL, 12.15 mmol). The reaction mixture was stirred at rt for 10 min, 1-piperazin-1-yl-ethanone (0.935 g, 7.29 mmol) was added at rt and the reaction mixture was stirred at rt for a further 2 h. The reaction was quenched with a saturated aqueous solution of NaHCO₃, extracted with CH₂Cl₂. The organic layer was washed with brine, dried over MgSO₄, filtered and evaporated under vacuum. Purification by flash chromatography on silica gel (CH₂Cl₂/MeOH, 95/5) gave the title compound (3.03 mg, 91% purity (HPLC), quantitative yield). ¹H-NMR (400 MHz, DMSO-d₆, 298 K): δ ppm 2.03 (br.s., 3H) 3.52 (br.s., 8H) 7.69-7.76 (m, 2H) 7.84 (s, 1H) 7.91 (d, 1H) 8.09 (d, 1H) 8.19-8.22 (m, 2H) 9.43 (s, 1H). MS: 439.6-441.8 [M+1]⁺, Rt⁽²⁾=1.02 min.

### 3-(6-Bromo-quinazolin-4-yl)-benzoic acid

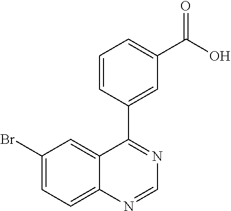 [SMILES: O=C(O)c1cccc(-c2ncnc3ccc(Br)cc23)c1]

To a solution of 3-(6-Bromo-quinazolin-4-yl)-benzoic acid ethyl ester (1.41 g, 4.11 mmol) in dioxane (45 mL) was added at rt a 1M aqueous solution of LiOH.H₂O (8.22 ml, 8.22 mmol) and the reaction mixture was stirred 3 h at rt. The reaction was quenched with a 1M aqueous solution of HCl (5 mL), the formed precipitate was filtered and dried under vacuum to gave the title compound (880 mg, 65% yield) as a light yellow solid. The filtrate was extracted with EtOAc, the organic layer was washed with brine, dried over MgSO₄, filtered and evaporated to give the title compound (555 mg, 35% yield) as a light yellow solid. The two isolated solids were combined to gave the title compound as a light yellow solid (880+550 mg=1.43 g, quantitative yield). MS: 331.0 [M+1]⁺, Rt⁽¹⁾=1.14 min.

### 3-(6-Bromo-quinazolin-4-yl)-benzoicacid ethyl ester

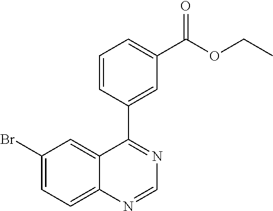 [SMILES: CCOC(=O)c1cccc(-c2ncnc3ccc(Br)cc23)c1]

To a mixture of 6-Bromo-4-chloro-quinazoline (2 g, 8.21 mmol), 3-(ethoxycarbonyl)phenyl-boronic acid (1.673 g, 8.62 mmol), Pd(PPh₃)₂Cl₂ (0.288 g, 0.411 mmol) and K₃PO₄ (2.62 g, 12.32 mmol) was added 16 mL of acetonitrile. The reaction mixture was flushed with argon, 2 mL of water was added, the tube was capped, heated to 100° C. for 15 min using a microwave oven and then cooled down to rt. The formed yellow solid was filtered, washed with ether and dried under vacuum to gave the title compound (1.54 g) as a yellow solid. The filtrate was diluted with EtOAc, the organic layer washed with brine, dried over MgSO₄, filtered and evaporated. The obtained residue was triturated in MeOH to afford the title compound as a yellow solid (580 mg). The two solids were combined to gave 2.12 g of the title compound as a yellow solid. ¹H-NMR (400 MHz, MeOD, 298 K): δ ppm 1.42 (t, 3H) 4.43 (q, 2H) 7.77 (t, 1H) 7.97-8.07 (m, 2H) 8.16 (dd, 1H) 8.22 (d, 1H) 8.29 (d, 1H) 8.41 (s, 1H) 9.34 (s, 1H). MS: 357.0-359.0 [M+1]⁺, Rt⁽¹⁾=1.52 min.

### 6-Bromo-4-chloro-quinazoline

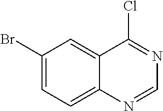 [SMILES: Clc1ncnc2ccc(Br)cc12]

6-Bromo-3H-quinazolin-4-one (20 g, 89 mmol) was suspended in 140 mL of POCl₃ and stirred 3 h at 140° C. The reaction mixture was concentrated under vacuum, the residue was dissolved in 500 mL of dry CH₂Cl₂ and neutralized with 200 g of solid NaHCO₃. The mixture was filtered and the filtrate evaporated under vacuum to gave the title compound (21 g, 95% yield) as a beige solid. ¹H-NMR (400 MHz, CDCl₃, 298 K): δ ppm 7.98 (d, 1H) 8.09 (d, 1H) 8.5 (s, 1H) 9.1 (s, 1H). MS: 243.0-244.9 [M+1]⁺, Rt⁽¹⁾=1.24 min.

### Example 2

### {3-[7-(2-Methoxy-pyrimidin-5-yl)-naphthalen-1-yl]-phenyl}-(4-methyl-piperazin-1-yl)-methanone

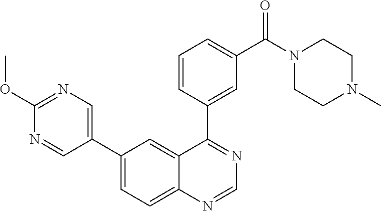 [SMILES: COc1ncc(-c2ccc3ncnc(-c4cccc(C(=O)N5CCN(C)CC5)c4)c3c2)cn1]

To a mixture of [3-(6-Bromo-quinazolin-4-yl)-phenyl]-(4-methyl-piperazin-1-yl)-methanone (50 mg, 0.122 mmol), 2-Methoxy-pyrimidine-5-boronic acid (22 mg, 0.146 mmol) and Pd(PPh₃)₄ (7 mg, 0.006 mmol) was added 2 mL of DME. The reaction mixture was flushed with argon and a 1M aqueous solution of Na₂CO₃ (0.243 mL, 0.243 mmol) was added and the vial was capped. The reaction mixture was heated to 140° C. for 10 min using a microwave oven then cooled down to rt, diluted with CH₂Cl₂, filtered through a Celite pad and portioned between H₂O/CH₂Cl₂. The organic layer was washed with brine, dried over MgSO₄, filtered and evaporated. Purification by preparative reverse phase Gilson HPLC and subsequent neutralization of the combined fractions over PL-HCO₃ MP gave the title compound (38 mg, 71% yield). ¹H-NMR (400 MHz, DMSO-d₆, 298 K): δ ppm 2.15 (s, 3H) 2.20-2.38 (m, 4H) 3.37-3.70 (m, 4H) 3.99 (s, 3H) 7.65 (d, 1H) 7.73 (t, 1H) 7.86 (s, 1H) 8.02 (d, 1H) 8.24 (d, 1H) 8.33 (s, 1H) 8.43 (d, 1H) 9.05 (s, 2H) 9.41 (s, 1H) MS: 441.1 [M+1]⁺, Rt⁽²⁾=0.75 min.

### [3-(6-Bromo-quinazolin-4-yl)-phenyl]-(4-methyl-piperazin-1-yl)-methanone

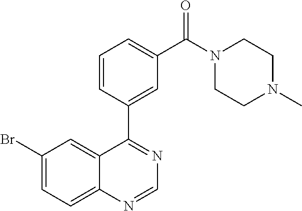 [SMILES: CN1CCN(C(=O)c2cccc(-c3ncnc4ccc(Br)cc34)c2)CC1]

To a mixture of 3-(6-Bromo-quinazolin-4-yl)-benzoic acid (2 g, 6.16 mmol) and HBTU (2.57 g, 6.78 mmol) was added DMF (15 mL) and DIPEA (2.26 mL, 12.95 mmol). The reaction mixture was stirred at rt for 10 min, 1-Methyl-piperazine (1.23 g, 12.33 mmol) was added at rt, followed by DIPEA (2.26 mL, 12.95 mmol) and the reaction mixture was stirred at rt for a further 5 min. The reaction was quenched with a saturated aqueous solution of NaHCO₃, extracted with AcOEt. The organic layer was washed with NaHCO₃, brine, dried over Na₂SO₄, filtered and evaporated under vacuum. Purification by flash chromatography on silica gel (CH₂Cl₂/MeOH, 99/1 to 90/10) gave the title compound (2.26 g, 90% purity (HPLC), 80% yield). ¹H-NMR (400 MHz, MeOD-d₄, 298 K): δ ppm 2.21 (s, 3H) 2.25-2.44 (m, 4H) 3.37-3.70 (m, 4H) 7.62-7.81 (m, 3H) 7.86-7.96 (m, 1H) 8.08 (d, 1H) 8.17-8.24 (m, 2H) 9.41 (s, 1H). MS: 411.4 [M+1]⁺, Rt⁽³⁾=1.38 min.

Examples 3 to 29, were prepared or can be prepared using procedures analogous to those used in example 1, using appropriate starting materials.

Examples 20, 21 and 22 were not neutralized after purification and were obtained as TFA salt.

| | | | MS(ES): | |
|---|---|---|---|---|
| Example | Structure/Name | Rt (min.) | [M + H]+ | 1H-NMR |
| 3 | 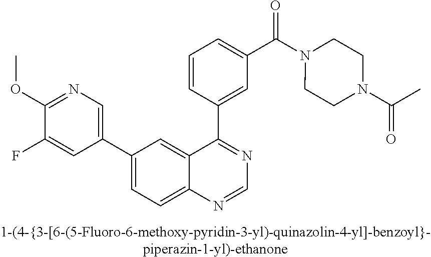 | 1.14 ⁽¹⁾ | 486.2 | ¹H-NMR (400 MHz, DMSO-d_{6,} 298 K): δ ppm 1.91-2.06 (br.s., 3 H) 3.38-3.73 (m, 8 H) 4.01 (s, 3 H) 7.69 (dt, 1 H) 7.75 (t, 1 H) 7.91 (br.s., 1 H) 8.02 (dt, 1 H) 8.22 (d, 2 H) 8.29 (d, 1 H) 8.42 (dd, 1 H) 8.45 (d, 1 H) 9.40 (s, 1 H) |
| 4 | 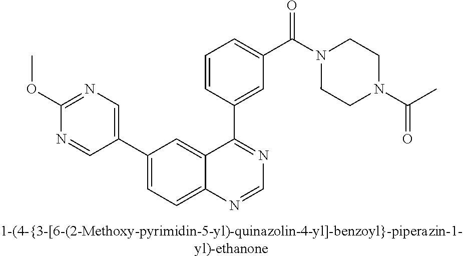 | 1.34 ⁽³⁾ | 469.5 | ¹H-NMR (400 MHz, DMSO-d_{6,} 298 K): δ ppm 2.15 (s, 3 H) 3.36-3.89 (m, 8 H) 4.09 (s, 3 H) 7.61-7.75 (m, 2 H) 7.90 (dd, 1 H) 7.92 (s, 1 H) 8.13 (dd, 1 H) 8.19 (d, 1 H) 8.29 (d, 1 H) 8.79 (s, 2 H) 9.44 (s, 1 H) |
| 5 | 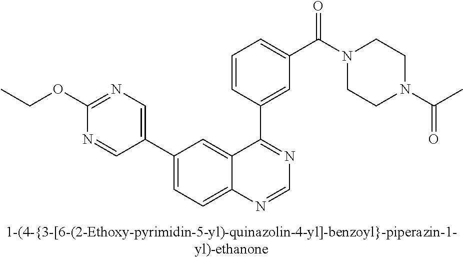 | 0.96 ⁽²⁾ | 483.6 | ¹H-NMR (400 MHz, DMSO-d_{6,} 298 K): δ ppm 1.37 (t, 3 H) 2.00 (br.s., 3 H) 3.35-3.76 (m, 8 H) 4.42 (q, 2 H) 7.69 (dt, 1 H) 7.74 (t, 1 H) 7.91 (br.s., 1 H) 8.02 (dt, 1 H) 8.23 (d, 1 H) 8.32 (d, 1 H) 8.43 (dd, 1 H) 9.04 (s, 2 H) 9.40 (s, 1 H) |
| 6 | 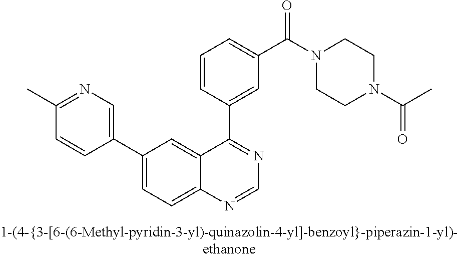 | 0.61 ⁽¹⁾ | 481.4 | ¹H-NMR (400 MHz, DMSO-d_{6,} 298 K): δ ppm 2.00 (br.s., 3 H) 2.66 (br.s., 3 H) 3.29-3.73 (m, 8 H) 7.67-7.79 (m, 3 H) 7.92 (br.s., 1 H) 8.03 (d, 1 H) 8.28 (d, 1 H) 8.38 (d, 1 H) 8.47 (dd, 1 H) 8.51 (br.s., 1 H) 9.08 (br.s., 1 H) 9.44 (s, 1 H) |
| 7 | 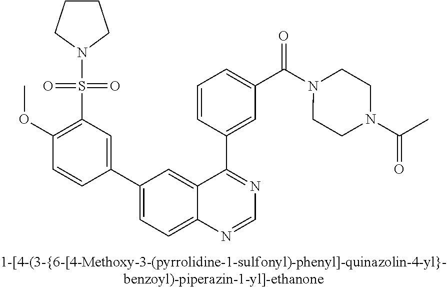 | 1.72 ⁽³⁾ | 600.3 | ¹H-NMR (400 MHz, DMSO-d_{6,} 298 K): δ ppm 1.83-1.91 (m, 4 H) 2.13 (br. s., 3 H) 3.36-3.45 (m, 4 H) 3.47-3.93 (m, 8 H) 4.01 (s, 3 H) 7.15 (d, 1 H) 7.66 (dt, 1 H) 7.70 (t, 1 H) 7.77 (dd, 1 H) 7.90 (br. s., 1 H) 7.93 (dt, 1 H) 8.11-8.30 (m, 4 H) 9.40 (s, 1 H) |
| 8 | 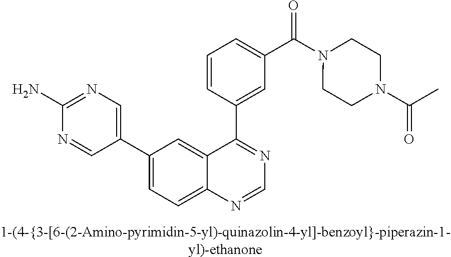 | 0.71 ⁽²⁾ | 454.5 | ¹H-NMR (400 MHz, DMSO-d_{6,} 298 K): δ ppm 2.00 (br.s., 3 H) 3.39-3.73 (m, 8 H) 6.94 (br.s., 2 H) 7.68 (d, 1 H) 7.74 (t, 1 H) 7.90 (br.s., 1 H) 8.01 (d, 1 H) 8.21 (d, 2 H) 8.37 (dd, 1 H) 8.70 (s, 2 H) 9.36 (s, 1 H) |
| 9 | 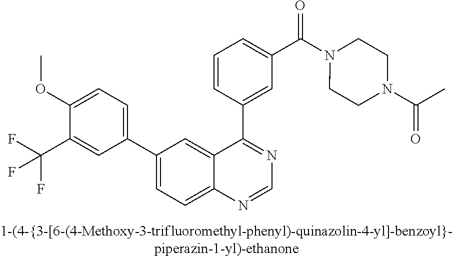 | 2.05 ⁽⁴⁾ | 544 | ¹H-NMR (400 MHz, DMSO-d_{6,} 298 K): δ ppm 1.80-2.10 (m, 3 H); 3.30-3.80 (m, 8 H), 3.96 (s, 3 H), 7.42 (d, 1 H), 7.70 (dd, 1 H), 7.75 (dd, 1 H), 7.92 (s, 1 H), 7.97 (d, 1 H), 8.02 (d, 1 H), 8.06 (d, 1 H), 8.21 (d, 1 H), 8.25 (d, 1 H), 8.44 (dd, 1 H), 9.39 (s, 1 H) |
| 10 | 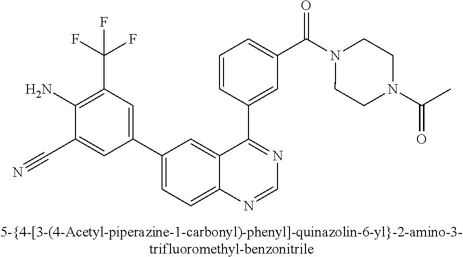 | 1.16 ⁽²⁾ | 545.7 | ¹H-NMR (400 MHz, DMSO-d_{6,} 298 K): δ ppm 1.97 (br.s., 3 H) 3.36-3.76 (m, 8 H) 6.55 (br.s., 2 H) 7.6-7.78 (m, 2 H) 7.91 (br.s., 1 H) 7.99-8.07 (m, 2 H) 8.16 (d, 1 H) 8.24 (d, 1 H) 8.29 (br.s., 1 H) 8.41 (dd, 1 H) 9.37 (s, 1 H) |
| 11 | 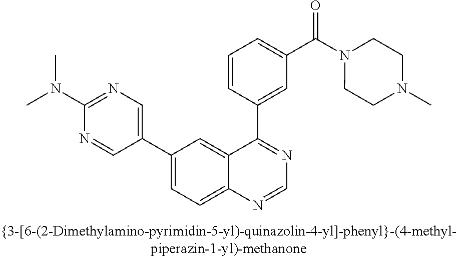 | 0.81 ⁽¹⁾ | 454.4 | ¹H-NMR (400 MHz, DMSO-d_{6,} 298 K): δ ppm 2.16 (s, 3 H) 2.20-2.45 (m, 4 H) 3.18 (s, 6 H) 3.38-3.70 (m, 4 H) 7.65 (d, 1 H) 7.73 (t, 1 H) 7.84 (s, 1 H) 8.00 (d, 1 H) 8.16-8.20 (m, 2 H) 8.37 (d, 1 H) 8.78 (s, 2 H) 9.35 (s, 1 H) |
| 12 | 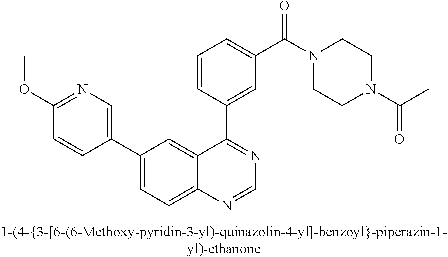 | 2.82 ⁽⁵⁾ | 468.2 | ¹H-NMR (400 MHz, MeOD-d_{4,} 298 K): δ ppm 2.13 (m, 3 H) 3.49-3.90 (m, 8 H) 3.97 (s, 3 H) 6.92 (d, 1 H) 7.75 (dt., 1 H) 7.78 (m, 1 H) 7.93 (s, 1 H) 7.98-8.04 (m, 2 H) 8.19 (d, 1 H) 8.23 (d, 1 H) 8.32 (dd, 1 H) 8.46 (d, 1 H) 9.31 (s, 1 H) |
| 13 | 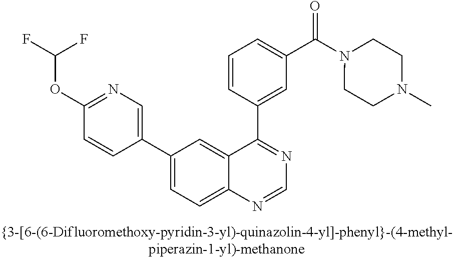 | 1.00 ⁽²⁾ | 476.1 | ¹H-NMR (400 MHz, DMSO-d_{6,} 298 K): δ ppm 2.15 (s, 3 H) 2.19-2.39 (m, 4 H) 3.39-3.68 (m, 4 H) 7.25 (d, 1 H) 7.65 (dt, 1 H) 7.73 (t, 1 H) 7.78 (t, 1 H) 7.84 (br.s., 1 H) 8.01 (dt, 1 H) 8.24 (d, 1 H) 8.30 (d, 1 H) 8.34 (dd, 1 H) 8.42 (dd, 1 H) 8.69 (d, 1 H) 9.41 (s, 1 H) |
| 14 | 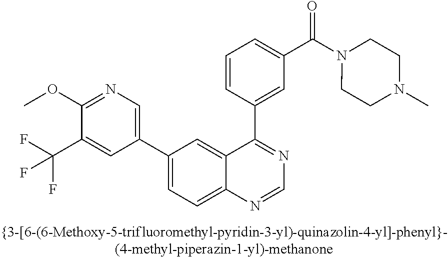 | 1.03 ⁽¹⁾ | 508.3 | ¹H-NMR (400 MHz, DMSO-d_{6,} 298 K): δ ppm 2.13 (s, 3 H) 2.15-2.41 (m, 4 H) 3.38-3.71 (m, 4 H) 4.05 (s, 3 H) 7.65 (dt, 1 H) 7.74 (t, 1 H) 7.85 (t, 1 H) 8.03 (dt, 1 H) 8.23 (d, 1 H) 8.33 (d, 1 H) 8.45-8.47 (dd, 2 H) 8.86 (d, 1 H) 9.41 (s, 1 H) |
| 15 | 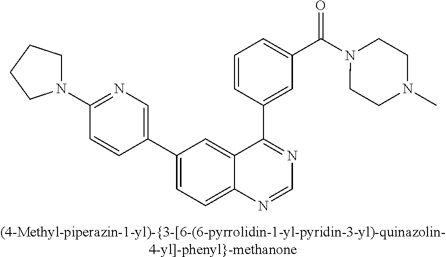 | 0.52 ⁽¹⁾ | 479.3 | ¹H-NMR (400 MHz, DMSO-d_{6,} 298 K): δ ppm 1.97 (m, 4 H) 2.20-2.65 (m, 7 H) 3.38-3.85 (m, 8 H) 6.56 (d, 1 H) 7.65 (d, 1 H) 7.74 (t, 1 H) 7.85 (s, 1 H) 7.89 (dd, 1 H) 7.99 (dt, 1 H) 8.14-8.16 (m, 2 H) 8.35 (dd, 1 H) 8.50 (d, 1 H) 9.33 (s, 1 H) |
| 16 | 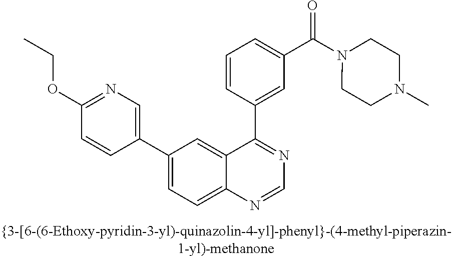 | 0.92 ⁽¹⁾ | 454.3 | ¹H-NMR (400 MHz, DMSO-d_{6,} 298 K): δ ppm 1.35 (t, 4 H) 2.21 (br.s., 3 H) 2.25-2.49 (m, 4 H) 3.40-3.75 (m, 4 H) 4.36 (q, 3 H) 6.93 (d, 1 H) 7.65 (dt, 1 H) 7.74 (t, 1 H) 7.84 (s, 1 H) 8.00 (dt, 1 H) 8.10 (dd, 1 H) 8.20-8.24 (m, 2 H) 8.39 (dd, 1 H) 8.57 (d, 1 H) 9.38 (s, 1 H) |
| 17 | 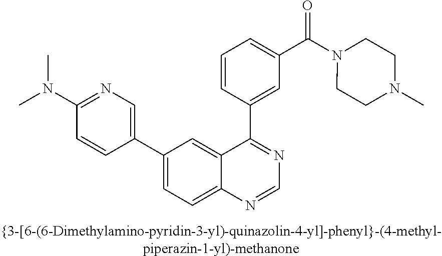 | 0.55 ⁽²⁾ | 453.2 | ¹H-NMR (400 MHz, DMSO-d_{6,} 298 K): δ ppm 2.16-2.44 (m, 7 H) 3.09 (s, 6 H) 3.40-3.70 (m, 4 H) 6.76 (d, 1 H) 7.66 (d, 1 H) 7.74 (t, 1 H) 7.84 (br.s., 1 H) 7.91 (dd, 1 H) 7.99 (d, 1 H) 8.15-8.17 (m, 2 H) 8.36 (dd, 1 H) 8.52 (d, 1 H) 9.34 (s, 1 H) |
| 18 | 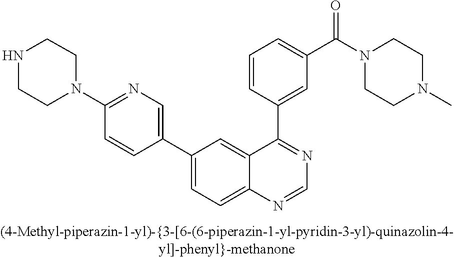 | 0.61 ⁽¹⁾ | 494.3 | ¹H-NMR (400 MHz, DMSO-d_{6,} 298 K): δ ppm 2.15 (s, 3 H) 2.18-2.42 (m, 4 H) 2.83 (t, 4 H) 3.40-3.72 (m, 8 H) 6.93 (d, 1 H) 7.65 (d, 1 H) 7.73 (t, 1 H) 7.82 (s, 1 H) 7.95 (dd, 1 H) 7.98 (dt, 1 H) 8.15-8.17 (m, 2 H) 8.37 (dd, 1 H) 8.53 (d, 1 H) 9.34 (s, 1 H) |
| 19 | 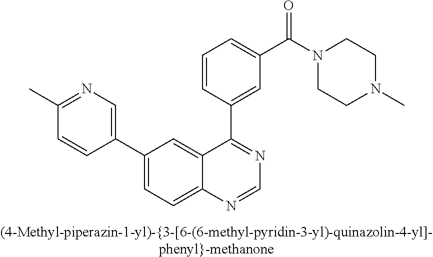 | 0.7 ⁽³⁾ | 242.2 | ¹H-NMR (400 MHz, DMSO-d_{6,} 298 K): δ ppm 2.17 (br.s., 3 H) 2.20-2.38 (m, 4 H) 2.53 (s, 3 H) 3.35-3.70 (m, 4 H) 7.39 (d, 1 H) 7.59 (d, 1 H) 7.73 (t, 1 H) 7.84 (s, 1 H) 8.00 (d, 1 H) 8.07 (d, 1 H) 8.23 (d, 1 H) 8.27 (s, 1 H) 8.40 (d, 1 H) 8.84 (s, 1 H) 9.40 (s, 1 H) |
| 20 | 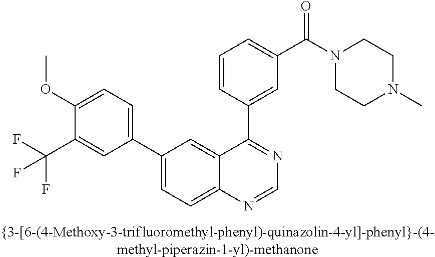 | 1.85 ⁽³⁾ | 507.1 | ¹H-NMR (400 MHz, DMSO-d_{6,} 298 K): δ ppm 2.08-2.43 (m, 7 H) 3.42 (br. s., 2 H) 3.62 (br. s., 2 H) 3.95 (s, 3 H) 7.41 (d, 1 H) 7.60-7.67 (m, 1 H) 7.73 (t, 1 H) 7.83 (s, 1 H) 7.94 (d, 1 H) 7.97-8.07 (m, 2 H) 8.16-8.25 (m, 2 H) 8.41 (dd, 1 H) 9.38 (s, 1 H) |
| 21 | 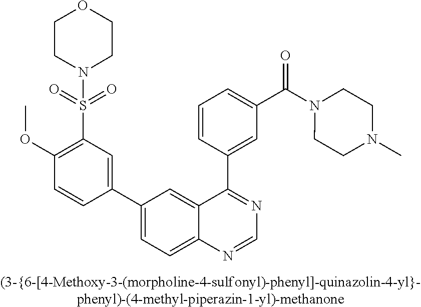 | 1.49 ⁽¹⁾ | 588.4 | ¹H-NMR (400 MHz, DMSO-d_{6,} 298 K): δ ppm 2.73 (d., 3 H) 3.02-3.18 (br.s., 6 H) 3.45-3.65 (m, 10 H) 3.99 (s, 3 H) 7.42 (d, 1 H) 7.75 (m, 2 H) 7.95 (s, 1 H) 8.05 (m, 3 H) 8.23 (m, 2 H) 8.40 (m, 1 H) 9.40 (s, 1 H) 11.00 (br.s., 1 H) |
| 22 | 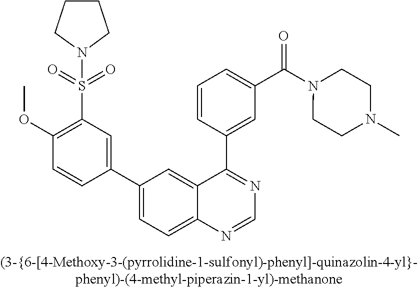 | 1.54 ⁽¹⁾ | 472.3 | ¹H-NMR (400 MHz, DMSO-d_{6,} 298 K): δ ppm 1.75 (m, 4 H) 2.73 (d, 3 H) 3.02-3.18 (br.s., 2 H) 3.25 (m, 4 H) 3.55-3.70 (br.s., 6 H) 3.99 (s, 3 H) 7.42 (d, 1 H) 7.75 (m, 2 H) 7.95 (s, 1 H) 8.05 (m, 3 H) 8.23 (m, 2 H) 8.40 (m, 1 H) 9.40 (s, 1 H) 10.95 (br.s., 1 H) |
| 23 | 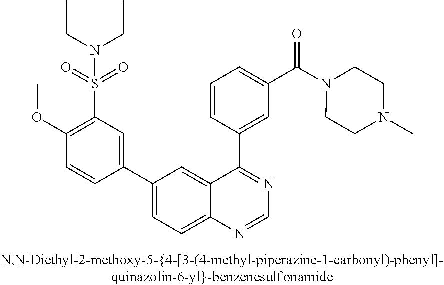 | 1.59 ⁽¹⁾ | 574.3 | ¹H-NMR (400 MHz, DMSO-d_{6,} 298 K): δ ppm 1.05 (t, 6 H) 2.75 (d, 3 H) 3.02-3.18 (br.s., 2 H) 3.30 (q, 4 H) 3.45-3.60 (br.s., 6 H) 3.95 (s, 3 H) 7.42 (d, 1 H) 7.75 (m, 2 H) 7.95 (s, 1 H) 8.05 (m, 3 H) 8.23 (m, 2 H) 8.40 (m., 1 H) 9.40 (s, 1 H) 11.35 (br.s., 1 H) |
| 24 | 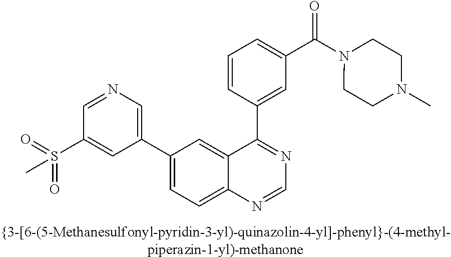 | 1.07 ⁽³⁾ | 488.2 | ¹H-NMR (400 MHz, DMSO-d_{6,} 298 K): δ ppm 2.17-2.47 (m, 7 H) 3.42 (s, 3 H) 3.45-3.81 (m, 4 H) 7.67 (d, 1 H) 7.74 (t, 1 H) 7.89 (s, 1 H) 8.05 (d, 1 H) 8.30 (d, 1 H) 8.44 (s, 1 H) 8.54 (d, 1 H) 8.67 (s, 1 H) 9.12 (s, 1 H) 9.33 (s, 1 H) 9.45 (s, 1 H) |
| 25 | 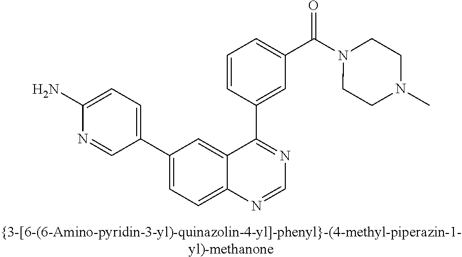 | 0.43 ⁽¹⁾ | 425.3 | ¹H-NMR (400 MHz, DMSO-d_{6,} 298 K): δ ppm 2.11 (s, 3 H) 2.13-2.34 (m, 4 H) 3.33-3.65 (m, 4 H) 6.22 (s, 2 H) 6.50 (d, 1 H) 7.59 (d, 1 H) 7.65-7.78 (m, 3 H) 7.92 (d, 1 H) 8.05-8.11 (m, 2 H) 8-25-8.30 (m, 2 H) 9.28 (s, 1 H) |
| 26 | 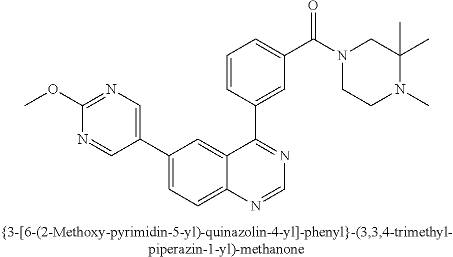 | 0.78 ⁽²⁾ | 469.2 | ¹H-NMR (400 MHz, DMSO-d_{6,} 298 K): δ ppm 0.82 (br.s., 3 H) 0.98 (br.s., 3 H) 2.10 (s, 3 H) 2.35 (br.s., 1 H) 2.49 (br.s., 1 H) 3.17 (br.s., 1 H) 3.35-3.65 (m, 3 H) 3.99 (s, 3 H) 7.65 (br.s., 1 H) 7.73 (t, 1 H) 7.85 (br.s., 1 H) 8.01 (d, 1 H) 8.24 (d, 1 H) 8.32 (br.s., 1 H) 8.43 (dd, 1 H) 9.04 (s, 2 H) 9.41 (s, 1 H) |
| 27 | 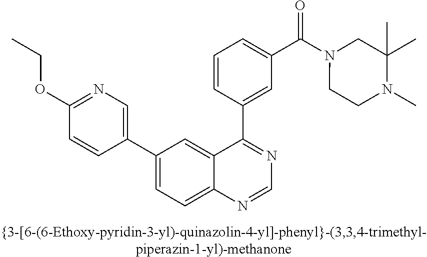 | 0.98 ⁽²⁾ | 482.2 | ¹H-NMR (400 MHz, DMSO-d_{6,} 298 K): δ ppm 0.82 (br.s., 3 H) 0.98 (br.s., 3 H) 1.34 (t, 3 H) 2.09 (s, 3 H) 2.34 (br.s., 2 H) 3.18 (br.s., 1 H) 3.41-3.65 (m, 3 H) 4.36 (q, 2 H) 6.92 (d, 1 H) 7.64 (br.s., 1 H) 7.73 (t, 1 H) 7.83 (br.s., 1 H) 7.99 (d, 1 H) 8.09 (dd, 1 H) 8.19-8.22 (m, 2 H) 8.38 (dd, 1 H) 8.56 (d, 1 H) 9.38 (s, 1 H) |
| 28 | 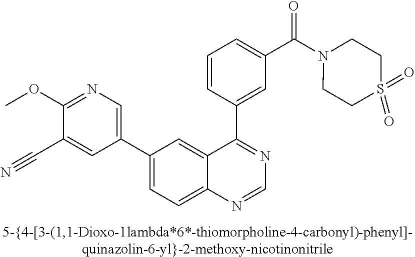 | 1.12 ⁽²⁾ | 500.5 | ¹H-NMR (400 MHz, DMSO-d_{6,} 298 K): δ ppm 3.12-3.29 (br.s., 4 H) 3.73-4.05 (br.s., 4 H) 4.07 (s, 3 H) 7.75 (d, 2 H) 8.02-8.06 (m, 2 H) 8.24 (d, 1 H) 8.35 (d, 1 H) 8.44 (dd, 1 H) 8.78 (d, 1 H) 8.91 (d, 1 H) 9.41 (s, 1 H) |
| 29 | 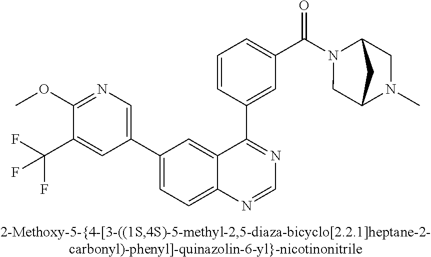 | 1.06 ⁽¹⁾ | 520.3 | ¹H-NMR (400 MHz, DMSO-d_{6,} 298 K): δ ppm 1.67 (m, 1 H) 1.78 (dd, 1 H) 2.22-2.26 (d, 3 H) 2.45-2.82 (m, 2 H) 3.24-3.48 (m, 3 H) 4.05 (s, 3 H) 4.30-4.61 (d, 1 H) 7.71-7.81 (m, 2 H) 7.90-8.00 (d, 1 H) 8.04 (m, 1 H) 8.23 (d, 1 H) 8.32 (dd, 1 H) 8.445 (d, 1 H) 8.47 (m, 1 H) 8.86 (dd, 1 H) 9.41 (s, 1 H) |
| 30 | 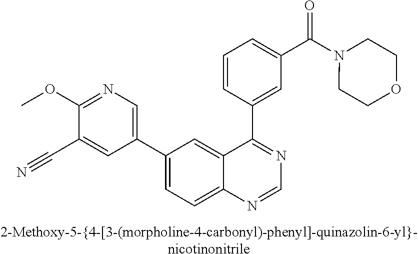 | 1.14 ⁽²⁾ | 452.5 | ¹H-NMR (400 MHz, DMSO-d_{6,} 298 K): δ ppm 3.40-3.73 (m, 8 H) 4.07 (s, 3 H) 7.68 (dt, 1 H) 7.74 (t, 1 H) 7.89 (br.s., 1 H) 8.02 (dt, 1 H) 8.23 (d, 1 H) 8.34 (d, 1 H) 8.44 (dd, 1 H) 8.79 (d, 1 H) 8.91 (d, 1 H) 9.41 (s, 1 H) |
| 31 | 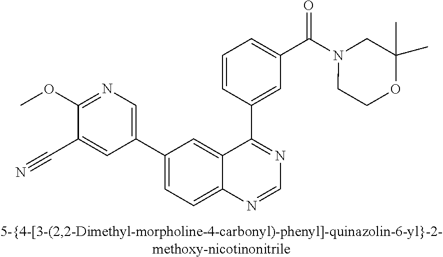 | 1.21 ⁽²⁾ | 480.6 | ¹H-NMR (400 MHz, DMSO-d_{6,} 298 K): δ ppm 1.06 (br.s., 3 H) 1.18 (br.s., 3 H) 3.37-3.73 (m, 6 H) 3.91 (s, 3 H) 6.95 (d, 1 H) 7.67 (br.s., 1 H) 7.74 (t, 1 H) 7.87 (br.s., 1 H) 8.00 (d, 1 H) 8.12 (dd, 1 H) 8.21 (d, 1 H) 8.24 (d, 1 H) 8.39 (dd, 1 H) 8.59 (d, 1 H) 9.39 (s, 1 H) |
| 32 | 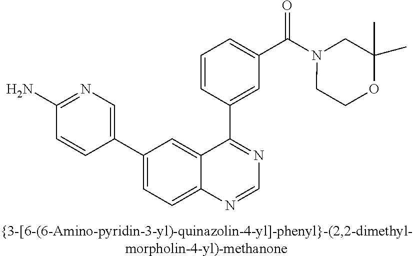 | 0.82 ⁽¹⁾ | 440.4 | ¹H-NMR (400 MHz, DMSO-d_{6,} 298 K): δ ppm 1.05-1.23 (m, 6 H) 3.40-3.72 (m, 6 H) 6.24 (s, 2 H) 6.55 (d, J = 8.56 Hz, 1 H) 7.67 (br. s., 1 H) 7.71-7.75 (m, 1 H) 7.75-7.81 (m, 1 H) 7.86 (br. s., 1 H) 7.98 (d, J = 7.34 Hz, 1 H) 8.10-8.16 (m, 2 H) 8.30-8.37 (m, 2 H) 9.33 (s, 1 H) |
| 33 | 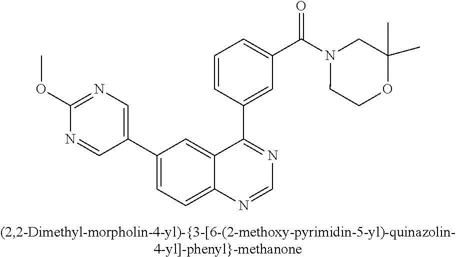 | 1.09 ⁽¹⁾ | 456.3 | ¹H-NMR (400 MHz, DMSO-d_{6,} 298 K): δ ppm 1.00-1.23 (m, 6 H) 3.35-3.72 (m, 6 H) 3.99 (s, 3 H) 7.68 (br. s., 1 H) 7.74 (t, J = 7.58 Hz, 1 H) 7.89 (br. s., 1 H) 8.02 (d, J = 7.34 Hz, 1 H) 8.24 (d, J = 8.80 Hz, 1 H) 8.33 (d, J = 1.71 Hz, 1 H) 8.44 (dd, J = 8.68, 1.83 Hz, 1 H) 9.05 (s, 2 H) 9.41 (s, 1 H) |

⁽¹⁾ LC methode 1,
⁽²⁾ LC methode 2,
⁽³⁾ LC methode 3,
⁽⁴⁾ LC methode 4,
⁽⁵⁾ LC methode 5

### Example 34

### 2-Methoxy-5-{4-[3-((R)-3-methyl-piperazine-1-carbonyl)-phenyl]-quinazolin-6-yl}-nicotinonitrile

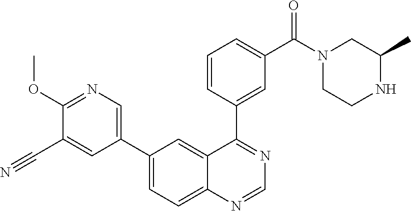 [SMILES: COc1ncc(-c2ccc3ncnc(-c4cccc(C(=O)N5CCN[C@H](C)C5)c4)c3c2)cc1C#N]

To a mixture (R)-4-[3-(6-bromo-quinazolin-4-yl)-benzoyl]-2-methyl-piperazine-1-carboxylic acid tert-butyl ester (100 mg, 0.196 mmol), 2-methoxy-5-(4,4,5,5-tetramethyl-[1,3,2]dioxaborolan-2-yl)-nicotinonitrile (76 mg, 0.235 mmol, 80% purity) and Pd(PPh₃)₄ (11.30 mg, 0.009 mmol) was added 3 mL of DME. The reaction mixture was flushed with argon and a 1M aqueous solution of Na₂CO₃ (0.391 mL, 0.391 mmol) was added and the vial capped. The reaction mixture was heated to 140° C. for 10 min using a microwave oven then cooled down to room temperature, diluted with EtOAc, filtered through a Celite pad and portioned between H₂O/EtOAc. The organic layer was washed with brine, dried over MgSO₄, filtered and evaporated. The residue was dissolved in 2 ml of CH₂Cl₂ and TFA (0.301 mL, 3.91 mmol) was added. The reaction mixture was stirred at room temperature for 2 h. After this period of time, the mixture was concentrated and purified by preparative reverse phase Gilson HPLC and subsequent neutralization of the combined fractions over PL-HCO₃ MP gave the title compound (36 mg, 39% yield) as a white powder. ¹H-NMR (400 MHz, DMSO-d₆, 298 K): δ ppm 0.74-1.05 (br.s., 3H), 2.35-3.10 (m, 5H) 3.47-3.65 (m, 1H) 4.06 (s, 3H) 4.33 (br.s., 1H) 7.64 (dt, 1H) 7.73 (t, 1H) 7.84 (t, 1H) 8.00 (dt, 1H) 8.23 (d, 1H) 8.33 (d, 1 H) 8.43 (dd, 1H) 8.78 (br.s., 1H) 8.90 (d, 1H) 9.40 (s, 1H). MS: 464.6 [M+1]⁺, Rt⁽¹⁾=0.98 min.

### (R)-4-[3-(6-bromo-quinazolin-4-yl)-benzoyl]-2-methyl-piperazine-1-carboxylic acid tert-butyl ester

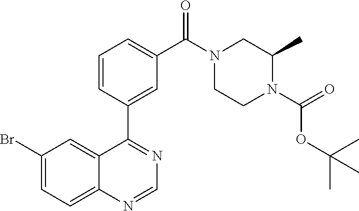 [SMILES: C[C@@H]1CN(C(=O)c2cccc(-c3ncnc4ccc(Br)cc34)c2)CCN1C(=O)OC(C)(C)C]

To a solution of 3-(6-bromo-quinazolin-4-yl)-benzoic acid (0.5 g, 1.519 mmol) in 15 mL of CH₂Cl₂ was added HBTU (0.634 g, 1.671 mmol) and DIPEA (0.796 mL, 4.56 mmol). The reaction mixture was stirred at room temperature for 30 minutes, (R)-2-methyl-piperazine-1-carboxylic acid tert-butyl ester (0.365 g, 1.823 mmol) was added and the reaction mixture was stirred at ambient temperature for a further 2 h. The reaction was quenched with H₂O and extracted with CH₂Cl₂. The organic layer was washed with brine, dried over MgSO₄, filtered and evaporated under vacuum. Purification by flash chromatography on silica gel (CH₂Cl₂/MeOH, 95/5) gave the title compound (1 g, 89% purity, quantitative yield). MS: 511.2-513.1 [M+1]⁺, Rt⁽¹⁾=1.51 min.

Examples 35 was prepared using procedures analogous to those used in example 34, using appropriate starting materials.

| | | | MS(ES): | |
|---|---|---|---|---|
| Example | Structure/Name | Rt (min.) | [M + H]⁺ | 1H-NMR |
| 35 | 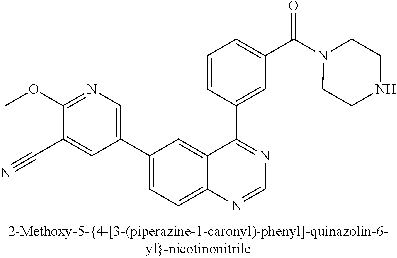 [SMILES: COc1ncc(-c2ccc3ncnc(-c4cccc(C(=O)N5CCNCC5)c4)c3c2)cc1C#N] | 0.98 ⁽²⁾ | 451.6 | ¹H-NMR (400 MHz, DMSO-d_{6,} 298 K): δ ppm 2.57-2.78 (m, 4 H) 3.35-3.62 (m, 4 H) 4.07 (s, 3 H) 7.65 (d, 1 H) 7.73 (t, 1 H) 7.85 (br.s., 1 H) 8.00 (d, 1 H) 8.23 (d, 1 H) 8.34 (d, 1 H) 8.43 (dd, 1 H) 8.79 (d, 1 H) 8.91 (d, 1 H) 9.41 (s, 1 H) |

⁽²⁾ LC methode 2

### Example 36

### 1-(4-{3-[6-(6-Methoxy-pyridin-3-yl)-quinazolin-4-yl]-benzoyl}-2,2-dimethyl-piperazin-1-yl)-ethanone

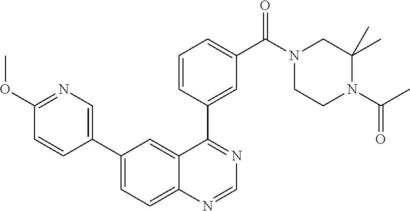 [SMILES: COc1ccc(-c2ccc3ncnc(-c4cccc(C(=O)N5CCN(C(C)=O)C(C)(C)C5)c4)c3c2)cn1]

To (3,3-dimethyl-piperazin-1-yl)-{3-[6-(6-methoxy-pyridin-3-yl)-quinazolin-4-yl]-phenyl}methanone (117.7 mg, 0.213 mmol) was added 4 mL of THF. Triethylamine (0.188 mL, 0.851 mmol) followed by acetyl chloride (0.023 mL, 0.319 mmol) were added. The reaction mixture was stirred for 5 min at room temperature. To the reaction mixture, addition of EtOAc. The organic layer was washed with NaHCO₃ sat. and brine, dried over Na₂SO₄, filtered and evaporated under vacuum. Purification by preparative reverse phase Gilson HPLC and subsequent neutralization of the combined fractions over PL-HCO₃ MP gave the title compound (82.7 mg, 78% yield) as a white powder. ¹H-NMR (400 MHz, DMSO-d₆, 298 K): δ ppm 1.16-1.53 (m, 6H) 1.86-2.05 (m, 3H) 3.46-3.75 (m, 6H) 3.90 (s, 3H) 6.88-7.00 (m, 1

H) 7.60-7.80 (m, 2H) 7.82-8.05 (m, 2H) 8.11 (dd, 1H) 8.18-8.27 (m, 2H) 8.38 (d, 1H) 8.58 (d, 1H) 9.38 (s, 1H). MS: 496.5 [M+1]⁺, Rt⁽³⁾=1.70 min.

### (3,3-Dimethyl-piperazin-1-yl)-{3-[6-(6-methoxy-pyridin-3-yl)-quinazolin-4-yl]-phenyl}-methanone

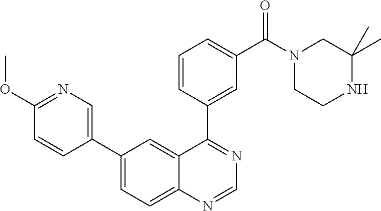 [SMILES: COc1ccc(-c2ccc3ncnc(-c4cccc(C(=O)N5CCNC(C)(C)C5)c4)c3c2)cn1]

To a mixture of [3-(6-bromo-quinazolin-4-yl)-phenyl]-(3,3-dimethyl-piperazin-1-yl)-methanone (111.9 mg, 0.263 mmol), 6-methoxypyridin-3-ylboronic acid (42.4 mg, 0.263 mmol) and Pd(PPh₃)₄ (30.4 mg, 0.026 mmol) was added 2.5 mL of acetonitrile. The reaction mixture was flushed with argon and a 1M aqueous solution of Na₂CO₃ (0.789 mL, 0.789 mmol) was added and the vial capped. The reaction mixture was heated to 130° C. for 20 min using a microwave oven then cooled down to rt, diluted with EtOAc, filtered through a Celite pad and portioned between aqueous NaHCO₃ sat./EtOAc. The organic layer was washed with brine, dried over Na₂SO₄, filtered and evaporated to give the crude compound (117.7 mg, 81% yield). MS: 454.5 [M+1]⁺, Rt⁽³⁾=1.40 min.

### [3-(6-Bromo-quinazolin-4-yl)-phenyl]-(3,3-dimethyl-piperazin-1-yl)-methanone

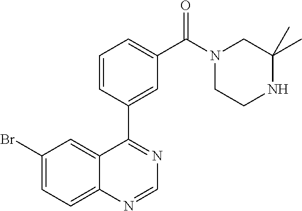 [SMILES: CC1(C)CN(C(=O)c2cccc(-c3ncnc4ccc(Br)cc34)c2)CCN1]

To a solution of 3-(6-bromo-quinazolin-4-yl)-benzoic acid (428.1 mg, 1.301 mmol) in 8 mL of DMF was added HBTU (543 mg, 1.431 mmol) and DIPEA (0.477 mL, 2.73 mmol). The reaction mixture was stirred at rt for 20 min, 2,2-dimethyl-piperazine (163 mg, 1.431 mmol) and DIPEA (0.477 mL, 2.73 mmol) were added at rt and the reaction mixture was stirred at rt overnight. The reaction was quenched with a saturated aqueous solution of NaHCO₃, extracted with EtOAc. The organic layer was washed with brine, dried over Na₂SO₄, filtered and evaporated under vacuum. Purification by flash chromatography on silica gel (CH₂Cl₂/MeOH, 99/1 to 90/10) gave the title compound (234.9 mg, >99% purity, 42.5% yield). MS: 427.1 [M+1]⁺, Rt⁽⁷⁾=1.17 min.

Examples 37 was prepared using procedures analogous to those used in example 36, using appropriate starting materials.

| | | Rt | MS(ES): | |
|---|---|---|---|---|
| Example | Structure/Name | (min.) | [M + H]+ | 1H-NMR |
| 37 | 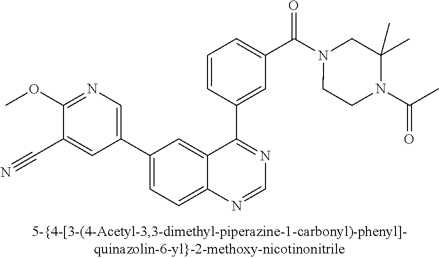 [SMILES: COc1ncc(-c2ccc3ncnc(-c4cccc(C(=O)N5CCN(C(C)=O)C(C)(C)C5)c4)c3c2)cc1C#N] | 1.782 ⁽³⁾ | 521.6 | ¹H-NMR (400 MHz, DMSO-d_{6,} 298 K): δ ppm 1.14-1.53 (m, 6 H) 1.81-2.05 (m, 3 H) 3.43-3.81 (m, 6 H) 4.06 (s, 3 H) 7.60-7.79 (m, 2 H) 7.81-7.96 (m, 1 H) 8.03 (d, 1 H) 8.23 (d, 1 H) 8.28-8.34 (m, 1 H) 8.41 (d, 1 H) 8.77 (d, 1 H) 8.88 (d, 1 H) 9.40 (s, 1 H) |

⁽³⁾ LC methode 3

### Example 38

### (2,5-Diaza-bicyclo[2.2.1]hept-2-yl)-{3-[6-(6-methoxy-pyridin-3-yl)-quinazolin-4-yl]-phenyl}-methanone

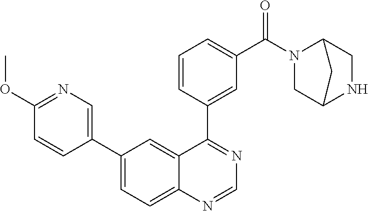 [SMILES: COc1ccc(-c2ccc3ncnc(-c4cccc(C(=O)N5CC6CC5CN6)c4)c3c2)cn1]

To a mixture of [3-(7-bromo-naphthalen-1-yl)-phenyl]-(2,5-diaza-bicyclo[2.2.1]hept-2-yl)-methanone (56.8 mg, 0.139 mmol), 6-methoxypyridin-3-ylboronic acid (23.35 mg, 0.153 mmol) and Pd(PPh₃)₄ (16.04 mg, 0.014 mmol) was added 1.5 mL of acetonitrile. The reaction mixture was flushed with argon and a 1M aqueous solution of Na₂CO₃ (0.416 mL, 0.416 mmol) was added and the vial capped. The reaction mixture was heated to 130° C. for 20 min using a microwave oven then cooled down to rt. After filtration, the mixture was directly purified by preparative reverse phase Gilson HPLC and subsequent neutralization of the combined fractions over PL-HCO₃ MP gave the title compound (26.7 mg, 44% yield) as a white powder. ¹H-NMR (400 MHz, DMSO-d₆, 298 K): δ ppm 1.50-1.85 (m, 2H) 2.73-3.05 (m, 2H) 3.35-3.75 (m, 3H) 3.91 (s, 3H) 4.35-4.70 (d, 1H) 6.95 (d, 1H) 7.69-7.78 (m, 2H) 7.91-8.01 (m, 2H) 8.10 (t, 1H) 8.19-8.24 (m, 2H) 8.39 (d, 1H) 8.59 (d, 1H) 9.38 (s, 1H). MS: 438.2 [M+1]⁺, Rt⁽³⁾=1.35 min.

### [3-(7-Bromo-naphthalen-1-yl)-phenyl]-(2,5-diaza-bicyclo[2.2.1]hept-2-yl)-methanone

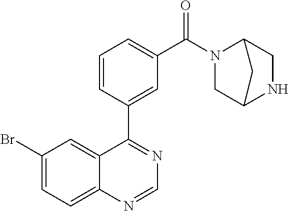 [SMILES: O=C(c1cccc(-c2ncnc3ccc(Br)cc23)c1)N1CC2CC1CN2]

To tert-butyl-5-(3-(6-bromoquinazolin-4-yl)benzoyl)-2,5-diazabicyclo[2.2.1]heptane-2-carboxylate (400.4 mg, 0.786 mmol) diluted in 10 mL of CH₂Cl₂, TFA (4 mL, 51.9 mmol) was added. The reaction mixture was stirred for 30 min at room temperature. The volatiles were evaporated and EtOAc was added. The organic layer was washed with an aqueous solution of NaHCO₃ and brine, dried over Na₂SO₄, filtered and evaporated to give the crude compound (158 mg, >99% purity, 49.1% yield). MS:409.0-410.9 [M+1]⁺, Rt⁽³⁾=1.22 min. tert-butyl 5-(3-(6-bromoquinazolin-4-yl)benzoyl)-2,5-diazabicyclo[2.2.1]heptane-2-carboxylate

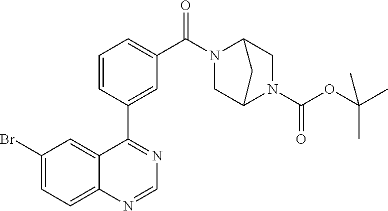 [SMILES: CC(C)(C)OC(=O)N1CC2CC1CN2C(=O)c1cccc(-c2ncnc3ccc(Br)cc23)c1]

To a solution of 3-(6-bromo-quinazolin-4-yl)-benzoic acid (310 mg, 0.942 mmol) in 8 mL of DMF was added HBTU (429 mg, 1.130 mmol) and DIPEA (0.3455 mL, 1.98 mmol). The reaction mixture was stirred at rt for 20 min. Tert-butyl 2,5-diazabicyclo[2.2.1]heptane-2-carboxylate (373 mg, 1.884 mmol) and DIPEA (0.3455 mL, 1.98 mmol) were added at rt and the reaction mixture was stirred for 10 min at rt. The reaction was quenched with a saturated aqueous solution of NaHCO₃, extracted with EtOAc. The organic layer was washed with brine, dried over Na₂SO₄, filtered and evaporated under vacuum. Purification by flash chromatography on silica gel (Heptane/EtOAc, 80/20 to 0/100) gave the title compound (400.4 mg, >99% purity, 83% yield). MS: 511.3 [M+1]⁺, Rt⁽³⁾=2.19 min.

Examples 39 was prepared using procedures analogous to those used for example 38, using appropriate starting materials.

| | | | MS(ES): | |
|---|---|---|---|---|
| Example | Structure/Name | Rt (min.) | [M + H]+ | 1H-NMR |
| 39 | 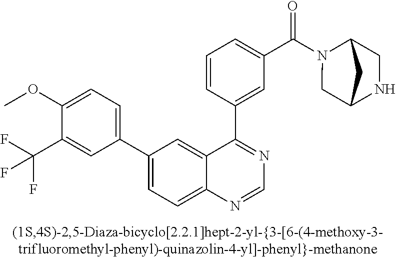 [SMILES: COc1ccc(-c2ccc3ncnc(-c4cccc(C(=O)N5C[C@@H]6C[C@H]5CN6)c4)c3c2)cc1C(F)(F)F] | 1.82 ⁽³⁾ | 505.2 | ¹H-NMR (400 MHz, DMSO-d_{6,} 298 K): δ ppm 1.41-1.62 (m, 1 H) 1.66-1.80 (m, 1 H) 2.65-2.87 (m, 1 H) 2.88-2.98 (m, 1 H) 3.19-3.27 (m, 1 H) 3.43-3.67 (m, 2 H) 3.95 (s, 3 H) 4.25-4.69 (m, 1 H) 7.40 (dd, 1 H) 7.61-7.80 (m, 2 H) 7.87-8.06 (m, 4 H) 8.15-8.27 (m, 2 H) 8.41 (dd, 1 H) 9.37 (d, 1 H) |

⁽³⁾ LC methode 3

### Example 40

### {3-[6-(5-Methyl-6-methylamino-pyridin-3-yl)-quinazolin-4-yl]-phenyl}-(4-methyl-piperazin-1-yl)-methanone

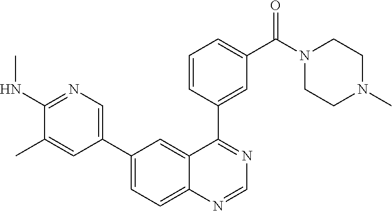 [SMILES: CNc1ncc(-c2ccc3ncnc(-c4cccc(C(=O)N5CCN(C)CC5)c4)c3c2)cc1C]

To a mixture of [3-(6-bromo-quinazolin-4-yl)-phenyl]-(4-methyl-piperazin-1-yl)-methanone (100 mg, 0.243 mmol), methyl-[3-methyl-5-(4,4,5,5-tetramethyl-[1,3,2]dioxaborolan-2-yl)-pyridin-2-yl]-carbamic acid tert-butylester (102 mg, 0.292 mmol) and Pd(PPh₃)₄ (14.05 mg, 0.012 mmol) was added 3 mL of DME. The reaction mixture was flushed with argon and a 1M aqueous solution of Na₂CO₃ (0.486 mL, 0.486 mmol) was added and the vial capped. The reaction mixture was heated to 140° C. for 10 min using a microwave oven then cooled down to rt, diluted with EtOAc, filtered through a Celite pad and portioned between H₂O/EtOAc. The organic layer was washed with brine, dried over MgSO₄, filtered and evaporated. The residue was dissolved in 3 ml of CH₂Cl₂ and TFA (0.562 mL, 7.29 mmol) was added. The reaction mixture was stirred at ambient temperature for 16 h. The reaction mixture was then concentrated and purified by preparative reverse phase Gilson HPLC and subsequent neutralization of the combined fractions over PL-HCO₃ MP gave the title compound (70 mg, 64% yield) as a white powder. ¹H-NMR (400 MHz, DMSO-d₆, 298 K): δ ppm 2.12 (s, 3H) 2.15 (s, 3H) 2.17-2.40 (m, 4H) 2.90 (d, 3H) 3.39-3.70 (m, 4H) 6.28 (q, 1H) 7.65 (br.s., 2H) 7.74 (t, 1H) 7.82 (s, 1H) 7.98 (d, 1H) 8.13 (d, 2H) 8.33 (d, 2H) 9.32 (s, 1H). MS: 453.3 [M+1]⁺, Rt⁽⁸⁾=1.25 min.

Examples 41 was prepared using procedures analogous to those used for example 40, using appropriate starting materials.

| | | | MS(ES): | |
|---|---|---|---|---|
| Example | Structure/Name | Rt (min.) | [M + H]+ | 1H-NMR |
| 41 | 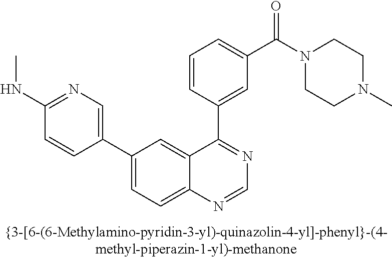 [SMILES: CNc1ccc(-c2ccc3ncnc(-c4cccc(C(=O)N5CCN(C)CC5)c4)c3c2)cn1] | 1.21 ⁽⁸⁾ | 439.3 | ¹H-NMR (400 MHz, DMSO-d_{6,} 298 K): δ ppm 2.16 (s, 3 H) 2.25 (br.s., 2 H) 2, 36 (br.s., 2 H) 2.82 (d, 3 H) 3.43 (br.s., 2 H) 3.64 (br.s., 2 H) 6.56 (d, 1 H) 6.82 (q, 1 H) 7.65 (dt, 1 H) 7.73 (t, 1 H) 7.80 (dd, 1 H) 7.82 (br.s., 1 H) 7.97 (dt, 1 H) 8.12 (d, 1 H) 8.14 (d, 1 H) 8.33 (dd, 1 H) 8.42 (d, 1 H) 9.32 (s, 1 H) |

⁽⁸⁾ LC methode 8

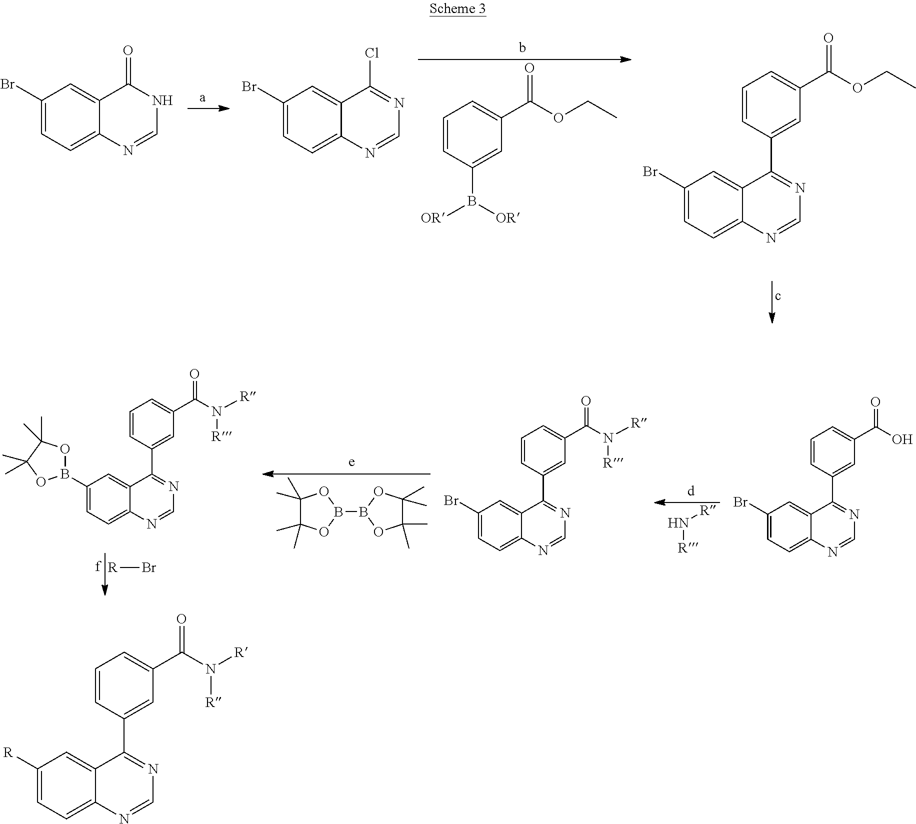

a) Chloronation of 6-Bromo-3H-quinazolin-4-one is performed under customary phophorus oxychoride condition by heating at reflux or 130° C. in diluted (such as in CH2Cl2) or neat phophorus oxychoride. b) Suzuki cross-coupling between 6-Bromo-4-chloro-quinazoline and 3-(ethoxycarbonyl)phenyl-boronic acid or 3-(ethoxycarbonyl)phenyl-boronate is performed under customary Suzuki conditions using palladium catalyst such as preferably Dichlorodiphenylphosphine palladium (PdCl₂(PPh₃)₂), aqueous base and organic solvent such as preferably acetonitrile. The reaction is preferably stirred at a temperature of approximately 100-120° C. in preferably a microwaves oven. The reaction may preferably carried out under an inert gas such as nitrogen or atrgon. c) Saponification of the carboxylic ester was performed under customary saponification conditions, using amoung the possible aqueous bases lithium hydroxyide is preferred and organic solvent such a preferably dioxane. The reation may preferably be carried out at room temperature. d) Condenation of a carboxylic acid with amines of the formula R″″NHR″ preferably takes place under customary condensation conditions. The reaction can be carried on by dissolving the carboxylic acid and the amine of formula R″′NHR″ in a suitable solvent , for example halogenated hydrocarbon, such as methylene chloride, N,N-dimethylformamide, N, N-dimethylacetamide, N-2-methyl-pyrrolidone, methylene chloride, or a mixture of two or more such solvents, and by the addition of a suitable base, for example triethylamine, diisopropylethylamine (DIPEA) or N-methylmorpholine and a suitable coupling agent that forms a reactive derivative of the carboxylic acid in situ, for example and preferably (2-(1H-Benzotriazole-1-yl)-1,1,3,3-tetramethyluronium hexafluorophosphate (HBTU). The reaction mixture is preferably stirred at a temperature of from approximately −20 to 50° C., especially from −5° C., e.g at 0° C. to room temperature. The reaction my preferably be carried out under an inert gas, e.g. nitrogenor argon. e) Formation of the boronate ester was performed using palladium catalyst such as preferably 1,1-Bis(diphenylphosphino)ferrocene[dichloropalladium (PdCl2(dppf)-CH₂Cl₂), aqueous base such as preferably potassium acetate organic solvent such as preferably dioxane and Bis-(pinacolato)-diboron. The reaction is preferably stirred at approximately 80° C. for several hours. f) Suzuki cross-coupling between aryl bromide (R—Br) and boronate is performed under customary Suzuki conditions using palladium catalyst such as preferably palladium tetrakis(triphenylphosphine) palladium (Pd(PPh₃)₄), aqueous base and organic solvent such as preferably acetonitrile. The reaction is preferably stirred at a temperature of approximately 100-120° C. in preferably a microwaves oven. The reaction may preferably carried out under an inert gas such as nitrogen or argon.

The final compounds described herein were according the general procedure described in scheme 3.

### Example 42

### {3-[6-(6-Ethoxy-5-trifluoromethyl-pyridin-3-yl)-quinazolin-4-yl]-phenyl}-(4-methyl-piperazin-1-yl)-methanone

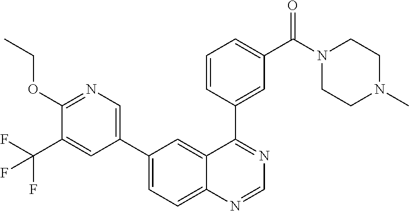 [SMILES: CCOc1ncc(-c2ccc3ncnc(-c4cccc(C(=O)N5CCN(C)CC5)c4)c3c2)cc1C(F)(F)F]

To a mixture of (4-methyl-piperazin-1-yl)-{3-[6-(4,4,5,5-tetramethyl-[1,3,2]dioxaborolan-2-yl)-quinazolin-4-yl]-phenyl}-methanone (100 mg, 0.218 mmol), 5-bromo-2-ethoxy-3-(trifluoromethyl)pyridine (70.7 mg, 0.262 mmol) and Pd(PPh₃)₄ (12.61 mg, 0.011 mmol) was added 2 mL of DME. The reaction mixture was flushed with argon and a 1M aqueous solution of Na₂CO₃ (0.436 mL, 0.436 mmol) was added and the vial capped. The reaction mixture was heated to 140° C. for 10 min using a microwave oven then cooled down to rt, diluted with EtOAc, filtered through a Celite pad and portioned between H₂O/EtOAc. The organic layer was washed with brine, dried over MgSO₄, filtered and evaporated. Purification by preparative reverse phase Gilson HPLC and subsequent neutralization of the combined fractions over PL-HCO₃ MP gave the title compound (70 mg, 61% yield) as a white powder.

¹H-NMR (400 MHz, DMSO-d₆, 298 K): δ ppm 1.37 (t, 3H) 2.13 (s, 3H) 2.17 (br.s., 2H) 2.34 (br.s., 2H) 3.43 (br.s., 2H) 3.62 (br.s., 2H) 4.53 (q, 2H) 7.65 (dt, 1H) 7.74 (t, 1H) 7.85 (t, 1 H) 8.02 (dt, 1H) 8.23 (d, 1H) 8.32 (d, 1H) 8.44-8.47 (m, 2H) 8.84 (d, 1H) 9.41 (s, 1H). MS⁽²⁾: 522.6 [M+1]⁺, Rt⁽²⁾=1.16 min.

### (4-Methyl-piperazin-1-yl)-{3-[6-(4,4,5,5-tetramethyl-[1,3,2]dioxaborolan-2-yl)-quinazolin-4-yl]-phenyl}-methanone

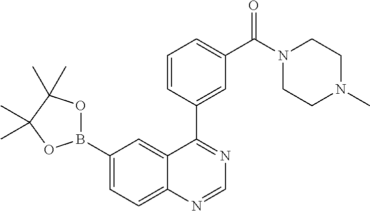

Bis-(pinacolato)-diboron (463 mg, 1.824 mmol), PdCl₂(dppf)-CH₂Cl₂ adduct (99 mg, 0.122 mmol) and KOAc (477 mg, 4.86 mmol) were placed into a a vial and degassed with stream of argon for 2 min. In a separate vial, [3-(6-Bromo-quinazolin-4-yl)-phenyl]-(4-methyl-piperazin-1-yl)-methanone (500 mg, 1.216 mmol) was dissolved in 10 mL of anhydrous dioxane. The dioxane solution of [3-(6-bromo-quinazolin-4-yl)-phenyl]-(4-methyl-piperazin-1-yl)-methanone was added to the “catalyst” vial and then heated at 80° C. for 2 h. After cooling to rt, 30 ml ethylacetate was added and the mixture was filtered trough a Celite pad. The dark filtrate was concentrated and then diluted in 30 ml heptane. A dark precipitate was formed and the mixture was filtered and the filtrate concentrated and then dried over high vacuum to give the title compound as a brown solid (710 mg, 50% purity, 55% yield). ¹H-NMR (400 MHz, CDCl3, 298 K): δ ppm 1.37 (s, 12H) 2.35 (s, 3H) 2.45 (br.s., 2H) 2.53 (br.s., 2H) 3.62 (br.s., 2H) 3.85 (br.s., 2H) 7.69 (d, 2H) 7.84 (br.s., 1H) 7.87 (m, 1H) 8.12 (d, 1H) 8.32 (dd, 1H) 8.52 (br.s., 1H) 9.41 (s, 1H). MS: 459.3 [M+1]⁺, Rt⁽¹⁾=1.0 min.

Examples 43 to 48, were prepared using procedures analogous to those used for example 42, using appropriate starting materials.

| | | | MS(ES): | |
|---|---|---|---|---|
| Example | Structure | Rt (min.) | [M + H]+ | 1H-NMR |
| 43 | 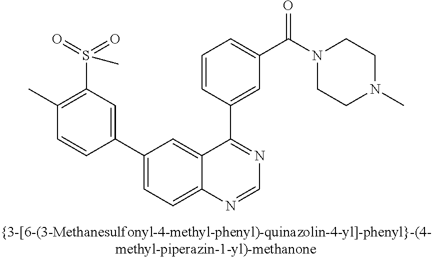 | 0.89 ⁽²⁾ | 501.5 | ¹H-NMR (400 MHz, DMSO-d_{6,} 298 K): δ ppm 2.13 (s, 3 H) 2.18 (m, 2 H) 2.32 (m, 2 H) 2.70 (s, 3 H) 3.29 (s, 3 H) 3.42 (m, 2 H) 3.60 (m, 2 H) 7.62 (d, 1 H) 7.66 (dt, 1 H) 7.73 (t, 1 H) 7.83 (br.s., 1 H) 7.97-8.04 (m, 2 H) 8.18 (d, 1 H) 8.22-8.29 (m, 2 H) 8.42 (dd, 1 H) 9.41 (s, 1 H) |
| 44 | 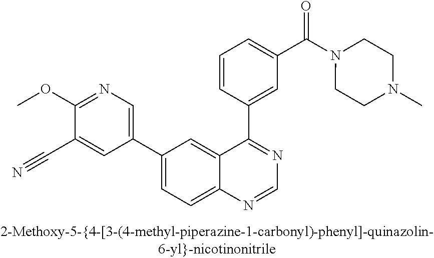 | 0.94 ⁽²⁾ | 465.1 | ¹H-NMR (400 MHz, DMSO-d_{6,} 298 K): δ ppm 2.15 (s, 3 H) 2.22 (br.s., 2 H) 2.35 (br.s., 2 H) 3.46 (br.s., 2 H) 3.62 (br.s., 2 H) 4.07 (s, 3 H) 7.65 (dt, H) 7.74 (t, 1 H) 7.83 (br.s., 1 H) 8.01 (dt, 1 H) 8.23 (d, 1 H) 8.34 (d, 1 H) 8.43 (dd, 1 H) 8.79 (d, 1 H) 8.90 (d, 1 H) 9.41 (s, 1 H) |
| 45 | 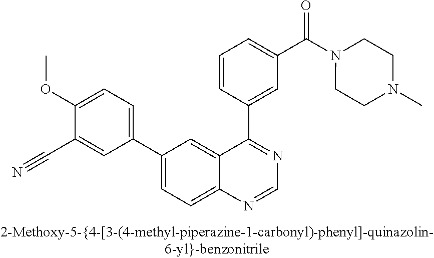 [SMILES: COc1ccc(-c2ccc3ncnc(-c4cccc(C(=O)N5CCN(C)CC5)c4)c3c2)cc1C#N] | 0.88 ⁽¹⁾ | 464.3 | ¹H-NMR (400 MHz, DMSO-d_{6,} 298 K): δ ppm 2.14 (s, 3 H) 2.19 (br.s., 2 H) 2.34 (br.s., 2 H) 3.44 (br.s., 2 H) 3.62 (br.s., 2 H) 3.98 (s, 3 H) 7.39 (d, 1 H) 7.65 (d, 1 H) 7.74 (t, 1 H) 7.82 (br.s., 1 H) 8.01 (d, 1 H) 8.08 (dd, 1 H) 8.20 (d, 1 H) 8.23 (d, 1 H) 8.26 (d, 1 H) 8.40 (dd, 1 H) 9.39 (s, 1 H) |
| 46 | 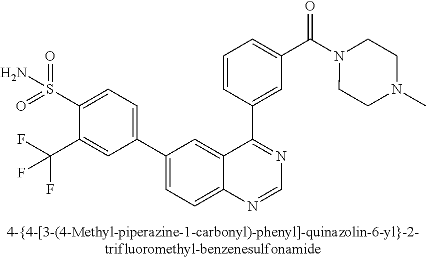 [SMILES: CN1CCN(C(=O)c2cccc(-c3ncnc4ccc(-c5ccc(S(N)(=O)=O)c(C(F)(F)F)c5)cc34)c2)CC1] | 1.49 ⁽¹⁾ | 556.2 | ¹H-NMR (400 MHz, DMSO-d_{6,} 298 K): δ ppm 2.09-2.41 (m, 7 H) 3.35-3.70 (m, 4 H) 7.68 (d, 1 H) 7.75 (t, 1 H) 7.85 (m, 2 H) 8.03 (d, 1 H) 8.21-8.30 (m, 4 H) 8.40 (s, 1 H) 8.50 (d, 1 H) 9.41 (s, 1 H) |
| 47 | 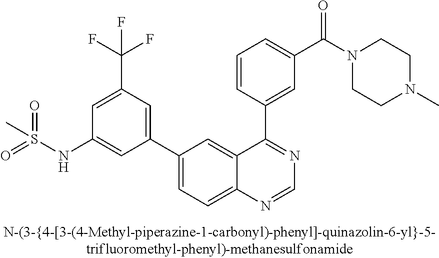 | 1.01 ⁽¹⁾ | 570.3 | ¹H-NMR (400 MHz, DMSO-d_{6,} 298 K): δ ppm 2.12 (m, 5 H) 2.32 (m, 2 H) 3.13 (s, 3 H) 3.35-3.68 (m, 4 H) 7.56 (s, 1 H) 7.66 (d, 1 H) 7.72-7.76 (m, 2 H) 7.83 (s, 3 H) 8.01 (d, 1 H) 8.26 (d, 2 H) 8.40 (d, 1 H) 9.43 (s, 1 H) |
| 48 | 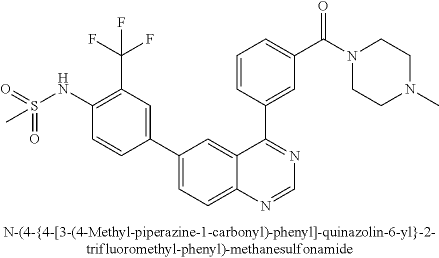 | 0.92 ⁽¹⁾ | 570.3 | ¹H-NMR (400 MHz, DMSO-d_{6,} 298 K): δ ppm 2.12 (s, 3 H) 2.13 (br.s., 2 H) 2.35 (br.s., 2 H) 3.13 (s, 3 H) 3.39 (br.s., 2 H) 3.61 (br.s., 2 H) 7.56 (br.s., 1 H) 7.66 (dt, 1 H) 7.73 (d, 1 H) 7.76 (br.s., 1 H) 7.83 (d, 2 H) 8.01 (dt, 1 H) 8.26 (d, 1 H) 8.27 (s, 1 H) 8.40 (dd, 1 H) 9.43 (s, 1 H) |

⁽¹⁾ LC methode 1,
⁽²⁾ LC methode 2

### Example 49

### 2-Methoxy-N,N-dimethyl-5-{4-[3-(4-methyl-piperazine-1-carbonyl)-phenyl]-quinazolin-6-yl}-benzamide

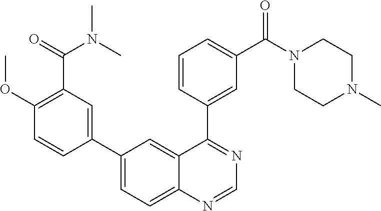 [SMILES: COc1ccc(-c2ccc3ncnc(-c4cccc(C(=O)N5CCN(C)CC5)c4)c3c2)cc1C(=O)N(C)C]

To a solution of 2-methoxy-5-{4-[3-(4-methyl-piperazine-1-carbonyl)-phenyl]-quinazolin-6-yl}-benzoic acid (50 mg, 0.084 mmol) in 2 mL of CH₂Cl₂ were added HBTU (38.1 mg, 0.101 mmol) and DIPEA (0.044 mL, 0.251 mmol). The reaction mixture was stirred at rt for 10 min, a solution of dimethyl amine in THF (2M) (0.210 mL, 0.419 mmol) was added at rt and the reaction mixture was stirred at rt for a further 30 min. The reaction was quenched with H₂O, extracted with CH₂Cl₂. The organic layer was washed with brine, dried over MgSO₄, filtered and evaporated under vacuum. Purification by reverse phase Gilson HPLC and subsequent neutralization of the combined fractions over PL-HCO₃ MP gave the title compound (25 mg, 58% yield) as a white powder. ¹H-NMR (400 MHz, DMSO-d₆, 298 K): δ ppm 2.15 (s, 3H) 2.22 (br.s., 2H) 2.36 (br.s., 2H) 2.79 (s, 3H), 2.99 (s, 3H) 3.41 (br.s., 2H) 3.62 (br.s., 2H) 3.86 (s, 3H) 7.22 (d, 1H) 7.56 (d, 1H) 7.65 (dt, 1H) 7.73 (t, 1H) 7.79 (dd, 1H) 7.82 (t, 1H) 7.98 (dt, 1H) 8.17 (d, 1H) 8.19 (s, 1H) 8.38 (dd, 1H) 9.37 (s, 1H). MS: 510.6 [M+1]⁺, Rt⁽²⁾=0.85 min.

### 2-Methoxy-5-{4-[3-(4-methyl-piperazine-1-carbonyl)-phenyl]-quinazolin-6-yl}-benzoic acid

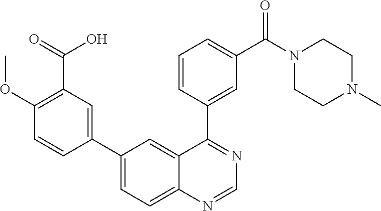 [SMILES: COc1ccc(-c2ccc3ncnc(-c4cccc(C(=O)N5CCN(C)CC5)c4)c3c2)cc1C(=O)O]

To a mixture of (4-methyl-piperazin-1-yl)-{3-[6-(4,4,5,5-tetramethyl-[1,3,2]dioxaborolan-2-yl)-quinazolin-4-yl]-phenyl}-methanone (300 mg, 0.655 mmol), 5-bromo-2-methoxy-benzoic acid (181 mg, 0.785 mmol) and Pd(PPh₃)₄ (37.8 mg, 0.033 mmol) was added 4 mL of DME. The reaction mixture was flushed with argon and a 1M aqueous solution of Na₂CO₃ (1.309 mL, 1.309 mmol) was added and the vial capped. The reaction mixture was heated to 140° C. for 10 min using a microwave oven then cooled down to rt, diluted with EtOAc, filtered through a Celite pad and concentrated. Purification by preparative reverse phase Gilson HPLC and the combined fractions gave the title compound (60 mg, 15% yield) as a white powder. ¹H-NMR (400 MHz, DMSO-d₆, 298 K): δ ppm 2.79 (s, 3H) 3.03-3.82 (m, 8H) 3.89 (s, 3H) 7.28 (d, 1 H) 7.72 (dt, 1H) 7.77 (t, 1H) 7.92 (dd, 2H) 7.98 (d, 1H) 8.05 (dt, 1H) 8.20-8.22 (m, 2H) 8.41 (dd, 1H) 9.39 (s, 1H). MS: 483.4 [M+1]⁺, Rt⁽¹⁾=0.75 min.

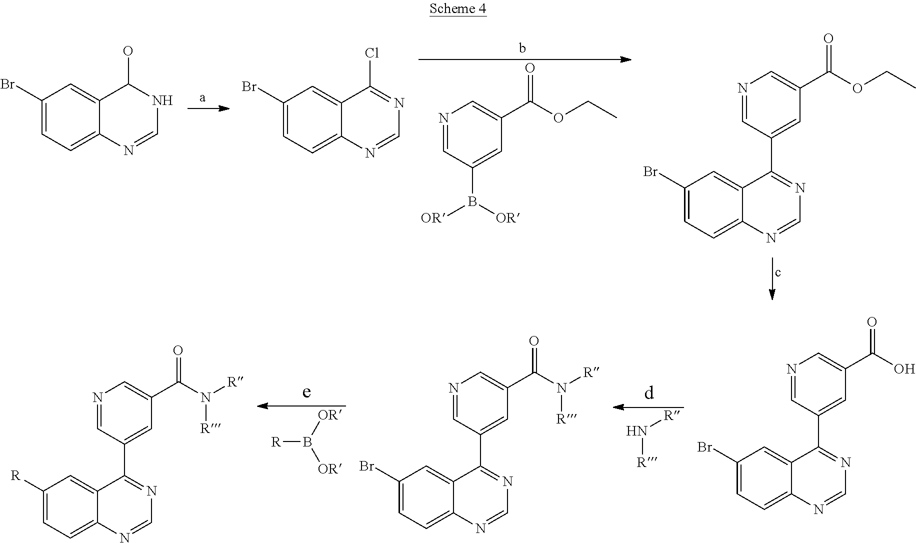

a) Chloronation of 6-Bromo-3H-quinazolin-4-one is performed under customary phophorus oxychoride condition by heating at reflux or 130° C. in diluted (such as in CH2Cl2) or neat phophorus oxychoride. b) Suzuki cross-coupling between 6-Bromo-4-chloro-quinazoline and 3-(ethoxycarbonyl)pyridyl-boronic acid or 3-(ethoxycarbonyl)pyridyl-boronate is performed under customary Suzuki conditions using palladium catalyst such as preferably Dichlorodiphenylphosphine palladium (PdCl₂(PPh₃)₂), aqueous base and organic solvent such as preferably acetonitrile. The reaction is preferably stirred at a temperature of approximately 100-120° C. in preferably a microwaves oven. The reaction may preferably carried out under an inert gas such as nitrogen or atrgon. c) Saponification of the carboxylic ester was performed under customary saponification conditions, using amoung the possible aqueous bases lithium hydroxyide is preferred and organic solvent such a preferably dioxane. The reaction may preferably be carried out at room temperature. d) Condenation of a carboxylic acid with amines of the formula R″′NHR″ preferably takes place under customary condensation conditions. The reaction can be carried on by dissolving the carboxylic acid and the amine of formula R″′NHR″ in a suitable solvent , for example halogenated hydrocarbon, such as methylene chloride, N,N-dimethylformamide, N,N-dimethylacetamide, N-2-methyl-pyrrolidone, methylene chloride, or a mixture of two or more such solvents, and by the addition of a suitable base, for example triethylamine, diisopropylethylamine (DIPEA) or N-methylmorpholine and a suitable coupling agent that forms a reactive derivative of the carboxylic acid in situ, for example and preferably (2-(1H-Benzotriazole-1-yl)-1,1,3,3-tetramethyluronium hexafluorophosphate (HBTU). The reaction mixture is preferably stirred at a temperature of from approximately −20 to 50° C., especially from −5° C. to 30° C., e.g at 0° C. to room temperature. The reaction my preferably be carried out under an inert gas, e.g. nitrogenor argon. e) Suzuki cross-coupling between aryl bromide and boronic acid or boronic acid derivatives such as boronate of formula R(OR′)₂ is performed under customary Suzuki conditions using palladium catalyst such as preferably palladium tetrakis(triphenylphosphine) palladium (Pd(PPh₃)₄), aqueous base and organic solvent such as preferably acetonitrile. The reaction is preferably stirred at a temperature of approximately 100-120° C. in preferably a microwaves oven. The reaction may preferably carried out under an inert gas such as nitrogen or argon.

The final compounds described herein were according the general procedure described in scheme 4.

### Example 50

### 1-(4-{5-[6-(5-Trifluoromethyl-pyridin-3-yl)-quinazolin-4-yl]-pyridine-3-carbonyl}-piperazin-1-yl)-ethanone

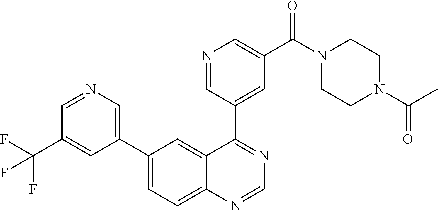 [SMILES: CC(=O)N1CCN(C(=O)c2cncc(-c3ncnc4ccc(-c5cncc(C(F)(F)F)c5)cc34)c2)CC1]

To a mixture of 1-{4-[5-(6-bromo-quinazolin-4-yl)-pyridine-3-carbonyl]-piperazin-1-yl}-ethanone (100 mg, 0.204 mmol, 90% purity (UPLC)), boronic acid 3-(4,4,5,5-tetramethyl-[1,3,2]dioxaborolan-2-yl)-5-trifluoromethyl-pyridine (80 mg, 0.204 mmol, 70% purity) and Pd(PPh₃)₄ (11.81 mg, 0.010 mmol) was added 2 mL of DME. The reaction mixture was flushed with argon and a 1M aqueous solution of Na₂CO₃ (0.409 mL, 0.409 mmol) was added and the vial capped. The reaction mixture was heated to 120° C. for 10 min using a microwave oven then cooled down to rt, diluted with EtOAc, filtered through a Celite pad and portioned between H₂O/EtOAc. The organic layer was washed with brine, dried over MgSO₄, filtered and evaporated. Purification by preparative reverse phase Gilson HPLC and subsequent neutralization of the combined fractions over PL-HCO₃ MP gave the title compound (55 mg, 53% yield) as a white powder. ¹H-NMR (400 MHz, DMSO-d₆, 298 K): δ ppm 1.96-2.1 (br.s., 3H) 3.41-3.70 (m, 8H) 8.31 (d, 1H) 8.40 (s, 1H) 8.50 (s, 1H) 8.56 (d, 1H) 8.69 (br.s., 1H) 8.90 (s, 1H) 9.04 (s, 1H) 9.20 (s., 1H) 9.35 (br.s., 1H) 9.49 (s, 1 H). MS: 507.6 [M+1]⁺, Rt⁽²⁾=0.93 min.

### 1-{4-[5-(6-Bromo-quinazolin-4-yl)-pyridine-3-carbonyl]-piperazin-1-yl}-ethanone

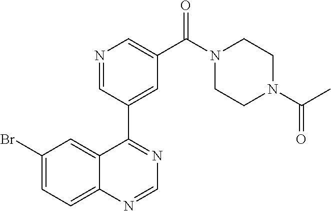 [SMILES: CC(=O)N1CCN(C(=O)c2cncc(-c3ncnc4ccc(Br)cc34)c2)CC1]

To a solution of 5-(6-bromo-quinazolin-4-yl)-nicotinic acid (1 g, 3.03 mmol) in 10 mL of CH₂Cl₂ was added HBTU (1.38 g, 3.63 mmol) and DIPEA (1.06 mL, 6.06 mmol). The reaction mixture was stirred at rt for 10 min, 1-piperazin-1-yl-ethanone (0.466 g, 3.63 mmol) was added at rt and the reaction mixture was stirred at rt for a further 3 h. The reaction was quenched with a saturated aqueous solution of NaHCO₃, extracted with CH₂Cl₂. The organic layer was washed with brine, dried over MgSO₄, filtered and evaporated under vacuum. Purification by flash chromatography on silica gel (CH₂Cl₂/MeOH, 95/5) gave the title compound (1.13 g, 90% purity, 76% yield). ¹H-NMR (400 MHz, DMSO-d₆, 298 K): δ ppm 2.04 (br.s., 3H) 3.41-3.70 (m, 8H) 8.12 (d, 1H) 8.24 (br.s., 2H) 8.31 (br.s., 1H) 8.89 (s, 1 H) 9.07 (s, 1H) 9.47 (s, 1H). MS: 440.4-442.4 [M+1]⁺, Rt⁽⁹⁾=1.48 min.

### 5-(6-Bromo-quinazolin-4-yl)-nicotinic acid

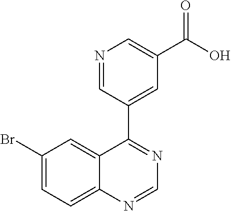 [SMILES: O=C(O)c1cncc(-c2ncnc3ccc(Br)cc23)c1]

To a solution of 5-(6-bromo-quinazolin-4-yl)-nicotinic acid ethyl ester (1.34 g, 3.74 mmol) in dioxane (45 mL) was added at rt a 1M aqueous solution of LiOH.H₂O (7.48 ml, 7.48 mmol) and the reaction mixture was stirred 1.5 h at rt. The reaction was quenched with a 1M aqueous solution of HCl (5 mL), the formed precipitate was filtered and dried under vacuum to gave the title compound as a light yellow solid. The filtrate was extracted with EtOAc, the organic layer was washed with brine, dried over MgSO₄, filtered and evaporated to give the title compound as a light yellow solid. The two isolated solids were combined to gave the title compound as a light yellow solid (1.1 g, 81% yield). MS: 330.5-332.5 [M+1]⁺, Rt⁽²⁾=0.97 min.

### 5-(6-Bromo-quinazolin-4-yl)-nicotinic acid ethyl ester

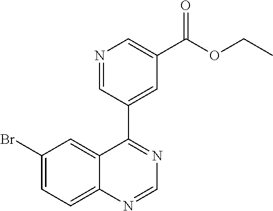 [SMILES: CCOC(=O)c1cncc(-c2ncnc3ccc(Br)cc23)c1]

To a mixture of 6-bromo-4-chloro-quinazoline (6 g, 23.41 mmol), boronic acid 5-(4,4,5,5-tetramethyl-[1,3,2]dioxaborolan-2-yl)-nicotinic acid ethyl ester (6.81 g, 24.58 mmol), Pd(PPh₃)₂Cl₂ (0.822 g, 1.17 mmol) and K₃PO₄ (7.45 g, 35.1 mmol) was added 96 mL of acetonitril. The reaction mixture was flushed with argon and 12 ml water was added and the vial capped. The reaction mixture was heated to 100° C. for 12 min using a microwave oven and then cooled down to rt. The mixture was quenched with water, extracted with dichloromethane. The organic layer was washed with brine, dried over MgSO₄, filtered through a Celite pad and evaporated. The obtained residue was triturated in MeOH to afford the title compound as a light orange solid (5.3 g, 95% purity, 60% yield). ¹H-NMR (400 MHz, DMSO-d₆, 298 K): δ ppm 1.38 (t, 3H) 4.41 (q, 2H) 8.1 (d, 1H) 8.25 (d, 2H) 8.65 (s, 1H) 9.22 (s, 1H) 9.32 (s, 1H) 9.48 (s, 1H). MS: 358.1-360.1 [M+1]⁺, Rt⁽¹⁾=1.28 min.

Examples 51 to 74, were prepared using procedures analogous to those used for example 50, using appropriate starting materials.

| | | | MS(ES): | |
|---|---|---|---|---|
| Example | Structure/Name | Rt (min.) | [M + H]+ | 1H-NMR |
| 51 | 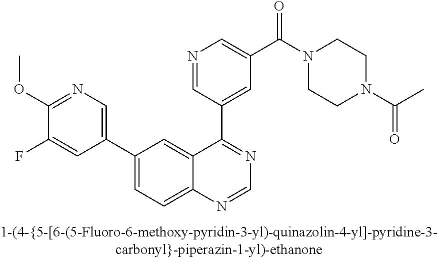 [SMILES: COc1ncc(-c2ccc3ncnc(-c4cncc(C(=O)N5CCN(C(C)=O)CC5)c4)c3c2)cc1F] | 0.95 ⁽¹⁾ | 487.3 | ¹H-NMR (400 MHz, DMSO-d_{6,} 298 K): δ ppm 1.85-2.03 (br.s., 3 H) 3.33-3.72 (m, 8 H) 3.96 (s, 3 H) 8.16-8.23 (m, 2 H) 8.27 (s, 1 H) 8.31 (s, 1 H) 8.41 (d, 1 H) 8.44 (s, 1 H) 8.84 (s, 1 H) 9.10 (s, 1 H) 9.39 (s, 1 H) |
| 52 | 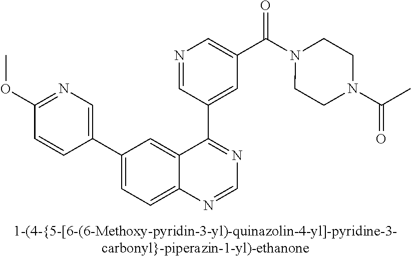 [SMILES: COc1ccc(-c2ccc3ncnc(-c4cncc(C(=O)N5CCN(C(C)=O)CC5)c4)c3c2)cn1] | 0.86 ⁽¹⁾ | 469.3 | ¹H-NMR (400 MHz, DMSO-d_{6,} 298 K): δ ppm 1.89-2.08 (br.s., 3 H) 3.37-3.75 (m, 8 H) 3.92 (s, 3 H) 6.97 (d, 1 H) 8.15-8.20 (d, 1 H) 8.23 (d, 1 H) 8.26 (d, 1 H) 8.37 (t, 1 H) 8.44 (dd, 1 H) 8.64 (d, 1 H) 8.88 (d, 1 H) 9.15 (d, 1 H) 9.43 (s, 1 H) |
| 53 | 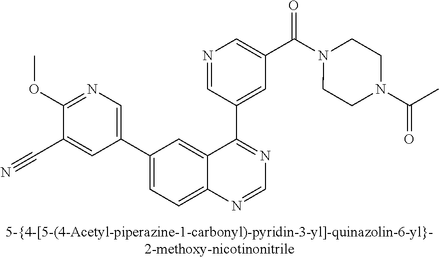 [SMILES: COc1ncc(-c2ccc3ncnc(-c4cncc(C(=O)N5CCN(C(C)=O)CC5)c4)c3c2)cc1C#N] | 0.93 ⁽¹⁾ | 494.3 | ¹H-NMR (400 MHz, DMSO-d_{6,} 298 K): δ ppm 1.82-2.02 (br.s., 3 H) 3.31-3.70 (m, 8 H) 4.03 (s, 3 H) 8.16-8.25 (m, 1 H) 8.32 (m, 2 H) 8.44 (m, 1 H) 8.80 (br.s., 1 H) 8.85 (d, 1 H) 8.92 (br.s., 1 H) 9.12 (d, 1 H) 9.41 (s, 1 H) |
| 54 | 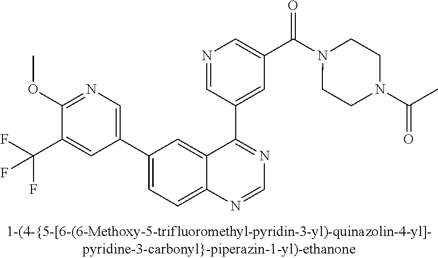 [SMILES: COc1ncc(-c2ccc3ncnc(-c4cncc(C(=O)N5CCN(C(C)=O)CC5)c4)c3c2)cc1C(F)(F)F] | 1.15 ⁽¹⁾ | 537.3 | ¹H-NMR (400 MHz, DMSO-d_{6,} 298 K): δ ppm 1.90-2.08 (br.s., 3 H) 3.36-3.74 (m, 8 H) 4.06 (s, 3 H) 8.26 (d, 1 H) 8.37-8.39 (m, 2 H) 8.49-8.52 (m, 2 H) 8.89 (d, 1 H) 8.91 (d, 1 H) 9.17 (d, 1 H) 9.45 (s, 1 H) |
| 55 | 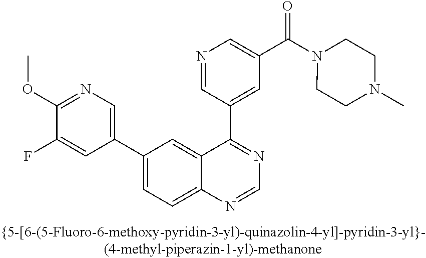 [SMILES: COc1ncc(-c2ccc3ncnc(-c4cncc(C(=O)N5CCN(C)CC5)c4)c3c2)cc1F] | 0.84 ⁽²⁾ | 459.1 | ¹H-NMR (400 MHz, DMSO-d_{6,} 298 K): δ ppm 2.16 (s, 3 H) 2.25 (br.s., 2 H) 2.37 (br.s., 2 H) 3.49 (br.s., 2 H) 3.65 (br.s., 2 H) 4.01 (s, 3 H) 8.21-8.26 (m, 2 H) 8.30-8.32 (m, 2 H) 8.44 (dd, 1 H) 8.47 (d, 1 H) 8.84 (d, 1 H) 9.13 (d, 1 H) 9.43 (s, 1 H) |
| 56 | 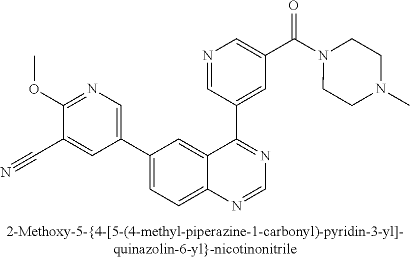 [SMILES: COc1ncc(-c2ccc3ncnc(-c4cncc(C(=O)N5CCN(C)CC5)c4)c3c2)cc1C#N] | 0.79 ⁽¹⁾ | 466.3 | ¹H-NMR (400 MHz, DMSO-d_{6,} 298 K): δ ppm 2.16 (s, 3 H) 2.25 (br.s., 2 H) 2.37 (br.s., 2 H) 3.49 (br.s., 2 H) 3.65 (br.s., 2 H) 4.07 (s, 3 H) 8.25 (d, 1 H) 8.30 (t, 1 H) 8.37 (d, 1 H) 8.47 (dd, 1 H) 8.83 (d, 1 H) 8.85 (d, 1 H) 8.95 (d, 1 H) 9.15 (d, 1 H) 9.45 (s, 1 H) |
| 57 | 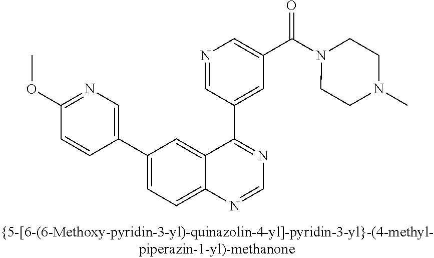 [SMILES: COc1ccc(-c2ccc3ncnc(-c4cncc(C(=O)N5CCN(C)CC5)c4)c3c2)cn1] | 1.10 ⁽¹⁾ | 441.4 | ¹H-NMR (400 MHz, DMSO-d_{6,} δ ppm 2.20 (br. s., 3 H) 2.26-2.48 (m, 4 H) 3.48 (br. s., 2 H) 3.66 (br. s., 2 H) 3.91 (s, 3 H) 6.96 (d, 1 H) 8.16 (dd, 1 H) 8.20-8.27 (m, 2 H) 8.31 (t, 1 H) 8.42 (dd, 1 H) 8.63 (d, 1 H) 8.84 (d, 1 H) 9.13 (d, 1 H) 9.42 (s, 1 H) |
| 58 | 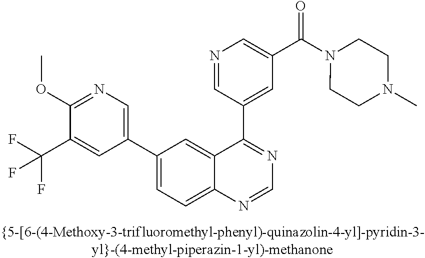 [SMILES: COc1ncc(-c2ccc3ncnc(-c4cncc(C(=O)N5CCN(C)CC5)c4)c3c2)cc1C(F)(F)F] | 1.57 ⁽¹⁾ | 508.4 | ¹H-NMR (400 MHz, DMSO-d_{6,} 298 K): δ ppm 2.01-2.44 (m, 7 H) 3.45 (br. s., 2 H) 3.64 (br. s., 2 H) 3.95 (s, 3 H) 7.41 (d, 1 H) 7.99 (s, 1 H) 8.07 (dd, 1 H) 8.18-8.26 (m, 2 H) 8.30 (s, 1 H) 8.44 (dd, 1 H) 8.84 (d, 1 H) 9.15 (d, 1 H) 9.42 (s, 1 H) |
| 59 | 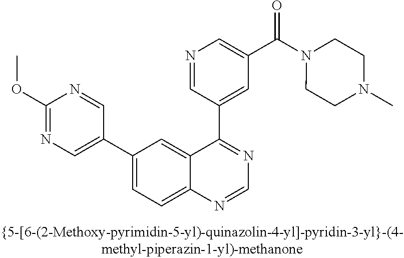 [SMILES: COc1ncc(-c2ccc3ncnc(-c4cncc(C(=O)N5CCN(C)CC5)c4)c3c2)cn1] | 0.56 ⁽¹⁾ | 442.3 | ¹H-NMR (400 MHz, DMSO-d_{6,} 298 K): δ ppm 2.17 (s, 3 H) 2.26 (br.s., 2 H) 2.38 (br.s., 2 H) 3.48 (br.s., 2 H) 3.66 (br.s., 2 H) 3.99 (s, 3 H) 8.27 (d, 1 H) 8.33 (t, 1 H) 8.36 (d, 1 H) 8.47 (dd, 1 H) 8.84 (d, 1 H) 9.09 (s, 2 H) 9.14 (d, 1 H) 9.45 (s, 1 H) |
| 60 | 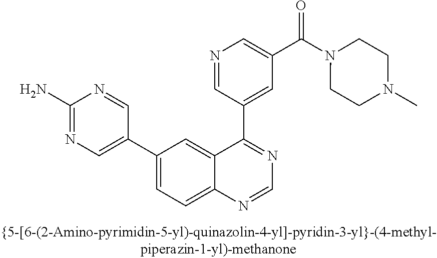 [SMILES: CN1CCN(C(=O)c2cncc(-c3ncnc4ccc(-c5cnc(N)nc5)cc34)c2)CC1] | 1.13 ⁽¹⁾ | 427.3 | ¹H-NMR (400 MHz, DMSO-d_{6,} 298 K): δ ppm 2.17 (s, 3 H) 2.27 (br.s., 2 H) 2.37 (br.s., 2 H) 3.48 (br.s., 2 H) 3.66 (br.s., 2 H) 6.97 (br.s., 2 H) 8.19 (d, 1 H) 8.22 (d, 1 H) 8.30 (t, 1 H) 8.40 (dd, 1 H) 8.72 (s, 2 H) 8.83 (d, 1 H) 9.13 (d, 1 H) 9.39 (s, 1 H) |
| 61 | 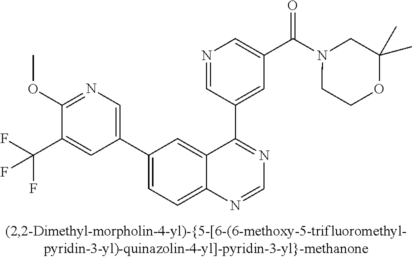 [SMILES: COc1ncc(-c2ccc3ncnc(-c4cncc(C(=O)N5CCOC(C)(C)C5)c4)c3c2)cc1C(F)(F)F] | 1.28 ⁽²⁾ | 524.5 | ¹H-NMR (400 MHz, DMSO-d_{6,} 298 K): δ ppm 1.04 (br.s., 3 H) 1.21 (br.s., 3 H) 3.37-3.75 (m, 6 H) 4.05 (s, 3 H) 8.24 (d, 1 H) 8.29-8.42 (m, 2 H) 8.43-8.54 (m, 2 H) 8.78-8.94 (m, 2 H) 9.16 (s, 1 H) 9.44 (s, 1 H) |
| 62 | 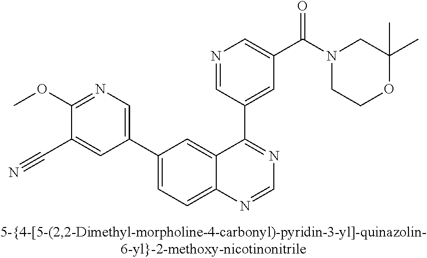 | 1.11 ⁽²⁾ | 481.6 | ¹H-NMR (400 MHz, DMSO-d_{6,} 298 K): δ ppm 1.06 (br.s., 3 H) 1.21 (br.s., 3 H) 3.43-3.73 (m, 6 H) 4.07 (s, 3 H) 8.25 (d, 1 H) 8.30-8.36 (m, 2 H) 8.46 (br.s., 1 H) 8.82-8.90 (m, 2 H) 8.94 (d, 1 H) 9.15 (d, 1 H) 9.45 (s, 1 H) |
| 63 | 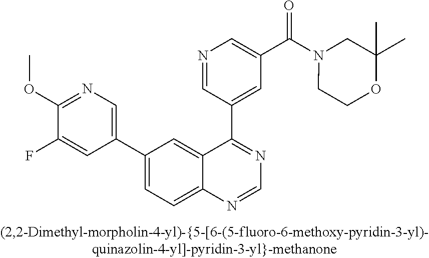 [SMILES: COc1ncc(-c2ccc3ncnc(-c4cncc(C(=O)N5CCOC(C)(C)C5)c4)c3c2)cc1F] | 1.12 ⁽²⁾ | 474.5 | ¹H-NMR (400 MHz, DMSO-d_{6,} 298 K): δ ppm 1.06 (br.s., 3 H) 1.21 (br.s., 3 H) 3.34-3.75 (m, 6 H) 4.01 (s, 3 H) 8.20-8.26 (m, 2 H) 8.30-8.40 (m, 2 H) 8.45 (d, 1 H) 8.47 (d, 1 H) 8.79-8.93 (m, 1 H) 9.14 (d, 1 H) 9.43 (s, 1 H) |
| 64 | 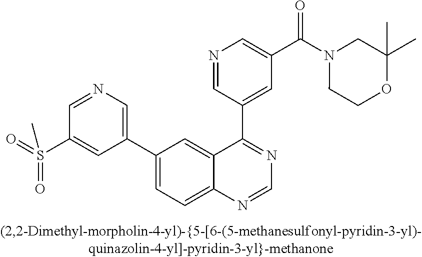 [SMILES: CC1(C)CN(C(=O)c2cncc(-c3ncnc4ccc(-c5cncc(S(C)(=O)=O)c5)cc34)c2)CCO1] | 0.94 ⁽²⁾ | 504.8 | ¹H-NMR (400 MHz, DMSO-d_{6,} 298 K): δ ppm 1.04 (br.s., 3 H) 1.21 (br.s., 3 H) 3.42 (s, 3 H) 3.43-3.74 (m, 6 H) 8.32 (d, 1 H) 8.34-8.43 (m, 1 H) 8.48 (d, 1 H) 8.53-8.60 (m, 1 H) 8.70 (br.s., 1 H) 8.81-8.92 (m, 1 H) 9.12 (d, 1 H) 9.18 (d, 1 H) 9.35 (d, 1 H) 9.49 (s, 1 H) |
| 65 | 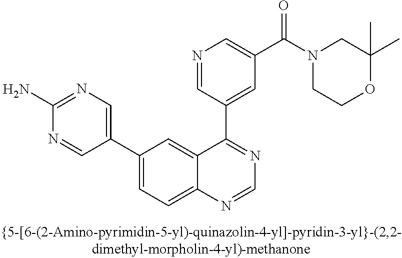 [SMILES: CC1(C)CN(C(=O)c2cncc(-c3ncnc4ccc(-c5cnc(N)nc5)cc34)c2)CCO1] | 0.82 ⁽²⁾ | 442.9 | ¹H-NMR (400 MHz, DMSO-d_{6,} 298 K): δ ppm 1.08 (br.s., 3 H) 1.21 (br.s., 3 H) 3.43-3.76 (m, 6 H) 6.95 (br.s., 2 H) 8.19 (d, 1 H) 8.22 (d, 1 H) 8.28-8.44 (m, 2 H) 8.72 (s, 2 H) 8.79-8.91 (m, 1 H) 9.13 (d, 1 H) 9.39 (s, 1 H) |
| 66 | 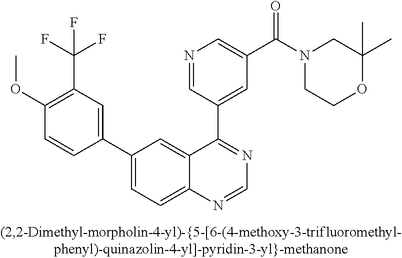 [SMILES: COc1ccc(-c2ccc3ncnc(-c4cncc(C(=O)N5CCOC(C)(C)C5)c4)c3c2)cc1C(F)(F)F] | 1.34 ⁽²⁾ | 523.7 | ¹H-NMR (400 MHz, DMSO-d_{6,} 298 K): δ ppm 1.04 (br.s., 3 H) 1.20 (br.s., 3 H) 3.39-3.74 (m, 6 H) 3.96 (s, 3 H) 7.40 (d, 1 H) 7.99 (br.s., 1 H) 8.06 (d, 1 H) 8.20-8.26 (m, 2 H) 8.28-8.47 (m, 2 H) 8.80-8.92 (m, 1 H) 9.16 (d, 1 H) 9.43 (s, 1 H) |
| 67 | 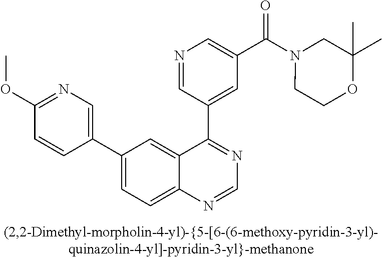 [SMILES: COc1ccc(-c2ccc3ncnc(-c4cncc(C(=O)N5CCOC(C)(C)C5)c4)c3c2)cn1] | 1.10 ⁽²⁾ | 456.8 | ¹H-NMR (400 MHz, DMSO-d_{6,} 298 K): δ ppm 1.06 (br.s., 3 H) 1.21 (br.s., 3 H) 3.41-3.74 (m, 6 H) 3.91 (s, 3 H) 6.96 (d, 1 H) 8.16 (d, 1 H) 8.21-8.26 (m, 2 H) 8.30-8.38 (m, 1 H) 8.43 (d, 1 H) 8.63 (d, 1 H) 8.80-8.92 (m, 1 H) 9.14 (d, 1 H) 9.43 (s, 1 H) |
| 68 | 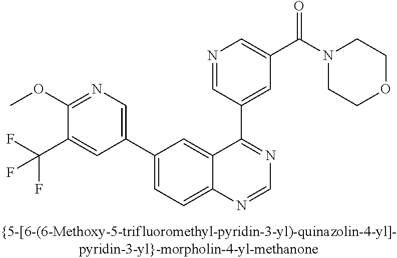 [SMILES: COc1ncc(-c2ccc3ncnc(-c4cncc(C(=O)N5CCOCC5)c4)c3c2)cc1C(F)(F)F] | 1.24 ⁽¹⁾ | 496.2 | ¹H-NMR (400 MHz, DMSO-d_{6,} 298 K): δ ppm 3.46-3.60 (m, 4 H) 3.60-3.73 (br.s., 4 H) 4.06 (s, 3 H) 8.25 (d, 1 H) 8.36-8.39 (m, 2 H) 8.49-8.53 (m, 2 H) 8.87 (d, 1 H) 8.91 (d, 1 H) 9.13 (d, 1 H) 9.45 (s, 1 H) |
| 69 | 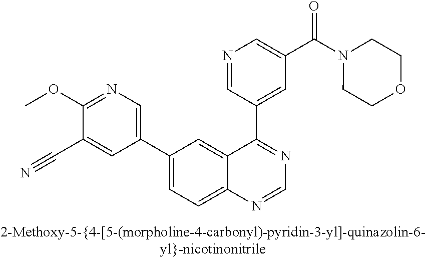 [SMILES: COc1ncc(-c2ccc3ncnc(-c4cncc(C(=O)N5CCOCC5)c4)c3c2)cc1C#N] | 1.00 ⁽¹⁾ | 453.3 | ¹H-NMR (400 MHz, DMSO-d_{6,} 298 K): δ ppm 3.46-3.75 (m, 8 H) 4.07 (s, 3 H) 8.25 (d, 1 H) 8.35 (t, 1 H) 8.37 (d, 1 H) 8.46-8.49 (dd, 1 H) 8.82 (d, 1 H) 8.88 (d, 1 H) 8.95 (d, 1 H) 9.15 (d, 1 H) 9.45 (s, 1 H) |
| 70 | 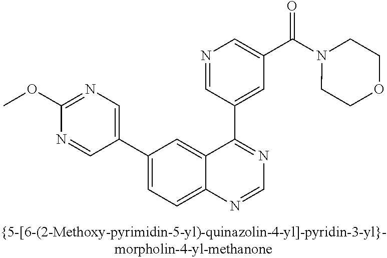 [SMILES: COc1ncc(-c2ccc3ncnc(-c4cncc(C(=O)N5CCOCC5)c4)c3c2)cn1] | 1.45 ⁽³⁾ | 429.3 | ¹H-NMR (400 MHz, DMSO-d_{6,} 298 K): δ ppm 3.37-3.90 (m, 8 H) 3.99 (s, 3 H) 8.26 (d, 1 H) 8.37 (m, 2 H) 8.48 (d, 1 H) 8.88 (s, 1 H) 9.09 (s, 2 H) 9.15 (s, 1 H) 9.45 (s, 1 H) |
| 71 | 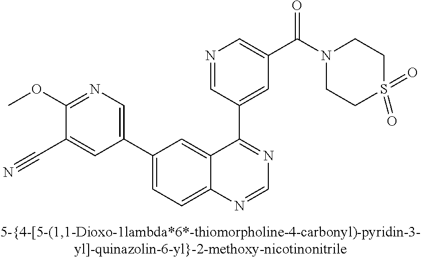 [SMILES: COc1ncc(-c2ccc3ncnc(-c4cncc(C(=O)N5CCS(=O)(=O)CC5)c4)c3c2)cc1C#N] | 1.02 ⁽²⁾ | 501.5 | ¹H-NMR (400 MHz, DMSO-d_{6,} 298 K): δ ppm 3.24 (br.s., 4 H) 3.87 (br.s., 2 H) 4.05 (br.s., 2 H) 4.07 (s, 3 H) 8.26 (d, 1 H) 8.39 (d, 1 H) 8.45 (t, 1 H) 8.49 (dd, 1 H) 8.83 (d, 1 H) 8.94 (d, 1 H) 8.96 (d, 1 H) 9.17 (d, 1 H) 9.46 (s, 1 H) |
| 72 | 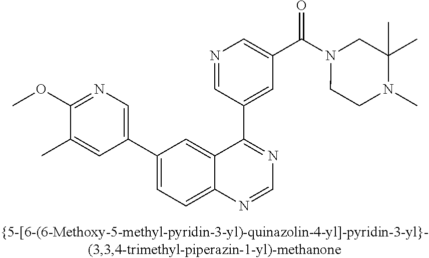 | 0.92 ⁽¹⁾ | 483.3 | ¹H-NMR (400 MHz, DMSO-d_{6,} 298 K): δ ppm 0.82 (br.s., 3 H) 0.99 (br.s., 3 H) 2.09 (m, 3 H) 2.22 (s, 3 H) 2.35 (br.s., 1 H) 2.50 (br.s., 1 H) 3.20 (br.s., 1 H) 3.39 (br.s., 1 H) 3.47 (br.s., 1 H) 3.67 (br.s., 1 H) 3.94 (s, 3 H) 8.01 (br.s., 1 H) 8.21-8.23 (d, 2 H) 8.29-8.31 (d, 1 H) 8.40 (d, 1 H) 8.43 (d, 1 H) 8.83 (d, 1 H) 9.13 (d, 1 H) 9.42 (s, 1 H) |
| 73 | 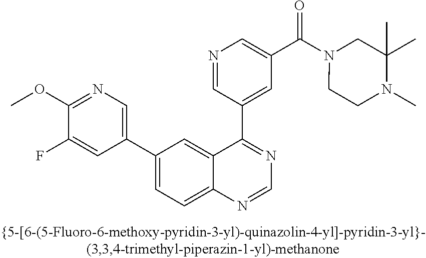 | 0.88 ⁽¹⁾ | 487.3 | ¹H-NMR (400 MHz, DMSO-d_{6,} 298 K): δ ppm 0.82 (br.s., 3 H) 0.99 (br.s., 3 H) 2.10 (br.s., 3 H) 2.37 (br.s., 1 H) 2.50 (br.s., 1 H) 3.21 (br.s., 1 H) 3.40 (br.s., 1 H) 3.49 (br.s., 1 H) 3.67 (br.s., 1 H) 4.01 (s, 3 H) 8.22-8.26 (m, 2 H) 8.30 (br.s., 2 H) 8.44 (d, 1 H) 8.47 (d, 1 H) 8.83 (d, 1 H) 9.13 (br.s., 1 H) 9.43 (s, 1 H) |
| 74 | 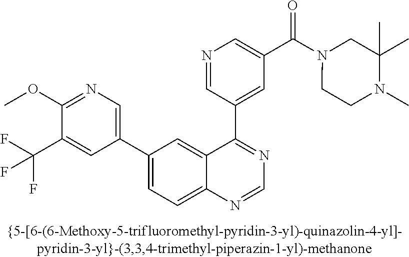 | 1.02 ⁽¹⁾ | 537.3 | ¹H-NMR (400 MHz, DMSO-d_{6,} 298 K): δ ppm 0.81 (br.s., 3 H) 0.99 (br.s., 3 H) 2.08 (br.s., 3 H) 2.33 (br.s., 1 H) 2.50 (br.s., 1 H) 3.19 (br.s., 1 H) 3.39 (br.s., 1 H) 3.47 (br.s., 1 H) 3.66 (br.s., 1 H) 4.05 (s, 3 H) 8.25 (d, 1 H) 8.27-8.38 (m, 2 H) 8.44-8.53 (m, 2 H) 8.78-8.85 (d, 1 H) 8.89 (br.s., 1 H) 9.15 (d, 1 H) 9.45 (s, 1 H) |

### Example 75

### {5-[6-(5-Methyl-6-methylamino-pyridin-3-yl)-quinazolin-4-yl]-pyridin-3-yl}-(4-methyl-piperazin-1-yl)-methanone

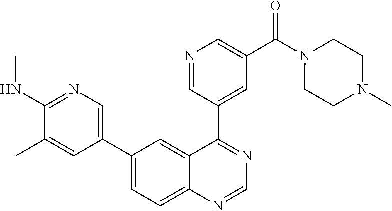

To a mixture of [5-(6-bromo-quinazolin-4-yl)-pyridin-3-yl]-(4-methyl-piperazin-1-yl)-methanone (100 mg, 0.243 mmol), tert-butyl methyl(3-methyl-5-(4,4,5,5-tetramethyl-1,3,2-dioxaborolan-2-yl)pyridin-2-yl) (107 mg, 0.291 mmol) and Pd(PPh3)₄ (14.01 mg, 0.012 mmol) was added 2 mL of DME. The reaction mixture was flushed with argon and a 1M aqueous solution of Na₂CO₃ (0.485 mL, 0.485 mmol) was added and the vial capped. The reaction mixture was heated to 120° C. for 10 min using a microwave oven then cooled down to rt, diluted with EtOAc, filtered through a Celite pad and portioned between H₂O/EtOAc. The organic layer was washed with brine, dried over MgSO₄, filtered and evaporated. The residue was dissolved in 2 ml of CH₂Cl₂ and TFA (0.374 mL, 4.85 mmol) was added. The reaction mixture was stirred at room temperature for 3 h. After this period of time, the mixture was concentrated and purified by preparative reverse phase Gilson HPLC and subsequent neutralization of the combined fractions over PL-HCO₃ MP gave the title compound (32 mg, 29% yield) as a white powder. ¹H-NMR (400 MHz, DMSO-d₆, 298 K): δ ppm 2.12 (s, 3H) 2.16 (s, 3H) 2.25 (br.s., 2H) 2.37 (br.s., 2H) 2.89 (d, 3H) 3.49 (br.s., 2H) 3.66 (br.s., 2H) 6.29 (q, 1H) 7.69 (d, 1H) 8.12 (d, 1H) 8.16 (d, 1H) 8.30 (t, 1H) 8.36-8.38 (m, 2H) 8.84 (d, 1H) 9.12 (d, 1H) 9.36 (s, 1H). MS: 454.2 [M+1]⁺, Rt⁽⁹⁾=1.21 min.

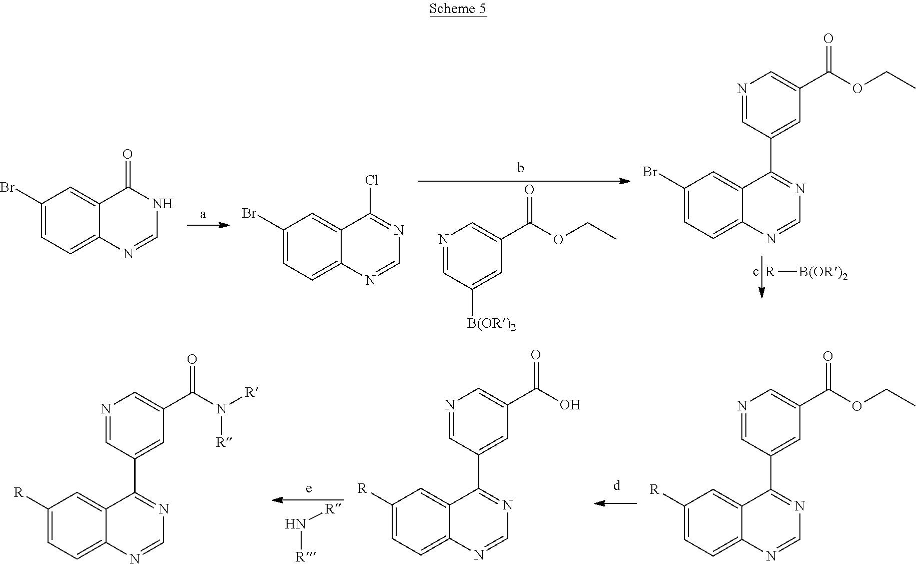

a) Chloronation of 6-Bromo-3H-quinazolin-4-one is performed under customary phophorus oxychoride condition by heating at reflux or 130° C. in diluted (such as in CH2Cl2) or neat phophorus oxychoride. b) Suzuki cross-coupling between 6-Bromo-4-chloro-quinazoline and 3-(ethoxycarbonyl)pyridyl-boronic acid or 3-(ethoxycarbonyl)pyridyl-boronate is performed under customary Suzuki conditions using palladium catalyst such as preferably Dichlorodiphenylphosphine palladium (PdCl₂(PPh₃)₂), aqueous base and organic solvent such as preferably acetonitrile. The reaction is preferably stirred at a temperature of approximately 100-120° C. in preferably a microwaves oven. The reaction may preferably carried out under an inert gas such as nitrogen or atrgon. c) Suzuki cross-coupling between aryl bromide and boronic acid or boronic acid derivatives such as boronate of formula R—B(OR′)₂ is performed under customary Suzuki conditions using palladium catalyst such as preferably palladium tetrakis(triphenylphosphine) palladium (Pd(PPh₃)₄), aqueous base and organic solvent such as preferably acetonitrile. The reaction is preferably stirred at a temperature of approximately 100-120° C. in preferably a microwaves oven. The reaction may preferably carried out under an inert gas such as nitrogen or argon. d) Saponification of the carboxylic ester was performed under customary saponification conditions, using among the possible aqueous bases lithium hydroxyide is preferred and organic solvent such a preferably dioxane. The reation may preferably be carried out at room temperature. e) Condenation of a carboxylic acid with amines of the formula R″′NHR″ preferably takes place under customary condensation conditions. The reaction can be carried on by dissolving the carboxylic acid and the amine of formula R″′NHR″ in a suitable solvent, for example halogenated hydrocarbon, such as methylene chloride, N,N-dimethylformamide, N,Ndimethylacetamide, N-2-methyl-pyrrolidone, methylene chloride, or a mixture of two or more such solvents, and by the addition of a suitable base, for example triethylamine, diisopropylethylamine (DIPEA) or N-methylmorpholine and a suitable coupling agent that forms a reactive derivative of the carboxylic acid in situ, for example and preferably (2-(1H-Benzotriazole-1-yl)-1,1,3,3-tetramethyluronium hexafluorophosphate (HBTU). The reaction mixture is preferably stirred at a temperature of from approximately −20 to 50° C., especially from −5° C. to 30° C., e.g at 0° C. to room temperature. The reaction my preferably be carried out under an inert gas, e.g. nitrogenor argon.

The final compounds described herein were according the general procedure described in scheme 5.

### Example 76

### {5-[6-(6-Methoxy-pyridin-3-yl)-quinazolin-4-yl]-pyridine-3-yl}-(4-methyl-[1,4]-diazepan-1-yl)-methanone

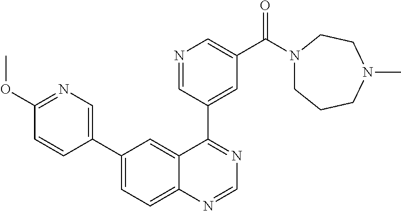 [SMILES: COc1ccc(-c2ccc3ncnc(-c4cncc(C(=O)N5CCCN(C)CC5)c4)c3c2)cn1]

To a solution of 5-[6-(6-methoxy-pyridin-3-yl)-quinazolin-4-yl]-nicotinic acid (100 mg, 0.279 mmol) in 2 mL of CH₂Cl₂ were added DIPEA (0.097 mL, 0.558 mmol) and propylphosphonic anhydride (solution on DMF, 50%) (0.244 mL, 0.419 mmol). The reaction mixture was stirred at rt for 30 min, 1-methyl-[1,4]-diazepane (65.7 mg, 0.557 mmol) was added and the reaction mixture was stirred at ambient temperature for a further 2 h. More 1-methyl-[1,4]-diazepane (49.27 mg, 0.418 mmol) and DIPEA (0.097 mL, 0.558 mmol) were added and the reaction mixture was stirred at ambient temperature for 16 h. The reaction was quenched with water and extracted with CH₂Cl₂. The organic layer was washed with brine, dried over MgSO₄, filtered and evaporated under vacuum. Purification by preparative reverse phase Gilson HPLC and subsequent neutralization of the combined fractions over PL-HCO₃ MP gave the title compound (54 mg, 43% yield) as a white powder. ¹H-NMR (400 MHz, DMSO-d₆, 298 K): δ ppm 1.77 (m, 1H) 1.86 (m, 1H) 2.17-2.28 (d, 3H) 2.44-2.56 (m, 3H) 2.66 (m, 1H) 3.50-3.59 (m, 2H) 3.63-3.72 (m, 2H) 3.92 (s, 3H) 6.96 (d, 1H) 8.14-8.18 (m, 1H) 8.22-8.24 (dd, 2H) 8.33 (dt, 1H) 8.43 (dd, 1H), 8.63 (t, 1H) 8.84 (dd, 1H) 9.12 (dd, 1H) 9.42 (s, 1H). MS: 455.2 [M+1]⁺, Rt⁽²⁾=0.79 min.

### 5-[6-(6-Methoxy-pyridin-3-yl)-quinazolin-4-yl]-nicotinic acid

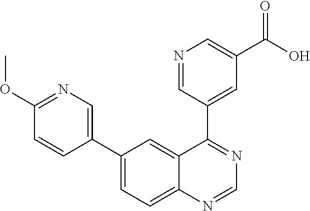 [SMILES: COc1ccc(-c2ccc3ncnc(-c4cncc(C(=O)O)c4)c3c2)cn1]

To a solution of 5-[6-(6-methoxy-pyridin-3-yl)-quinazolin-4-yl]-nicotinic acid ethyl ester (0.91 g, 2.19 mmol, 93% purity (HPLC)) in dioxane (30 mL) was added at rt a 1M aqueous solution of LiOH.H₂O (4.38 mL, 4.38 mmol) and the reaction mixture was stirred 3 hours at ambient temperature. The reaction was quenched with a 1M aqueous solution of HCl, the formed precipitate was filtered and dried under vacuum to gave the title compound (570 mg, 72% yield) as a light yellow solid. The filtrate was extracted with EtOAc, the organic layer was washed with brine, dried over MgSO₄, filtered and evaporated to give the title compound (205 mg, 27% yield) as a yellow solid. The two isolated solids were combined to gave the title compound (570+205 mg=775 mg, 99% yield). MS: 359.2 [M+1]⁺, Rt⁽¹⁾=0.96 min.

### 5-[6-(6-Methoxy-pyridin-3-yl)-quinazolin-4-yl]-nicotinic acid ethyl ester

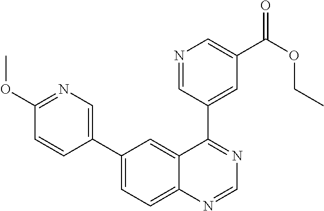 [SMILES: CCOC(=O)c1cncc(-c2ncnc3ccc(-c4ccc(OC)nc4)cc23)c1]

To a mixture of [5-(6-bromo-quinazolin-4-yl)-nicotinic acid ethyl ester (1 g, 2.79 mmol), 2-methoxy-5-pyridin boronic acid (0.448 g, 2.93 mmol) and Pd(PPh₃)₄ (0.161 mg, 0.140 mmol) was added 15 mL of DME. The reaction mixture was flushed with argon and a 1M aqueous solution of Na₂CO₃ (5.58 mL, 5.58 mmol) was added and the vial capped. The reaction mixture was heated to 120° C. for 20 min using a microwave oven then cooled down to rt, diluted with EtOAc, filtered through a Celite pad and portioned between H₂O/EtOAc. The organic layer was washed with brine, dried over MgSO₄, filtered and evaporated. The residue gave the title compound (910 mg, 93% purity, 78% yield). ¹H-NMR (400 MHz, DMSO-d₆, 298 K): δ ppm 1.37 (t, 3H) 3.91 (s, 3H) 4.42 (q, 2H) 6.96 (d, 1H) 8.14 (dd, 1H) 8.23-8.25 (m, 2 H) 8.43 (dd, 1H) 8.62 (d, 1H) 8.72 (t, 1H) 9.31 (dd, 2H) 9.43 (s, 1H). MS: 387.1 [M+1]⁺, Rt⁽²⁾=1.24 min.

Examples 77 to 83, were prepared using procedures analogous to those used for example 76, using appropriate starting materials.

| | | | MS (ES): | |
|---|---|---|---|---|
| Example | Structure/Name | Rt (min.) | [M + H]⁺ | 1H-NMR |
| 77 | 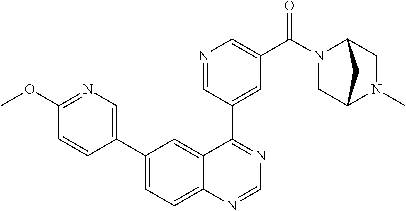 {5-[6-(6-Methoxy-pyridin-3-yl)-quinazolin-4-yl]- pyridin-3-yl}-((1S,4S)-5-methyl-2,5-diaza- bicyclo[2.2.1]hept-2-yl)-methanone | 0.78 ⁽²⁾ | 453.2 | ¹H-NMR (400 MHz, DMSO-d₆, 298 K): δ ppm 1.68-1.85 (m, 2 H) 2.25-2.27 (d, 3 H) 2.45-2.84 (m, 2 H) 3.28-3.56 (m, 3 H) 3.92 (s, 3 H) 4.35-4.64 (d, 1 H) 6.96 (d, 1 H) 8.14-8.19 (m, 1 H) 8.22-8.26 (m, 2 H) 8.33-8.47 (m, 2 H) 8.62- 8.64 (dd, 1 H) 8.90-8.97 (dd, 1 H) 9.15 (m, 1 H) 9.42 (s, 1 H) |
| 78 | 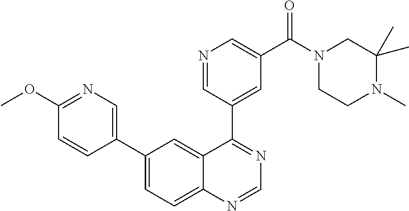 {5-[6-(6-Methoxy-pyridin-3-yl)-quinazolin-4-yl]- pyridin-3-yl}-(3,3,4-trimethyl-piperazin-1-yl)- methanone | 0.83 ⁽²⁾ | 469.2 | ¹H-NMR (400 MHz, DMSO-d₆, 298 K): δ ppm 0.84 (br.s., 3 H) 0.99 (br.s., 3 H) 2.10 (br.s., 3 H) 2.37 (br.s., 1 H) 2.50 (br.s., 1 H) 3.20 (br.s., 1 H) 3.40 (br.s., 1 H) 3.47 (br.s., 1 H) 3.68 (br.s., 1 H) 3.91 (s, 3 H) 6.92- 6.99 (m, 1 H) 8.16 (dd, 1 H) 8.22- 8.25 (m, 2 H) 8.29-8.32 (m, 1 H) 8.42 (dd, 1 H) 8.62 (d, 1 H) 8.78-8.87 (d, 1 H) 9.13 (d, 1 H) 9.42 (s, 1 H) |
| 79 | 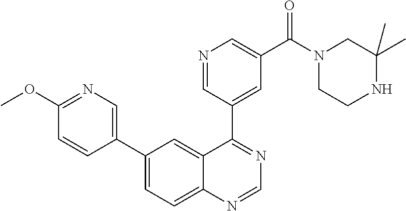 (3,3-Dimethyl-piperazin-1-yl)-{5-[6-(6-methoxy- pyridin-3-yl)-quinazolin-4-yl]-pyridin-3-yl}- methanone | 0.81 ⁽²⁾ | 455.2 | ¹H-NMR (400 MHz, DMSO-d₆, 298 K): δ ppm 0.91 (br.s., 3 H) 1.07 (br.s., 3 H) 2.69 (br.s., 1 H) 2.79 (br.s., 1 H) 3.18 (br.s., 1 H) 3.38 (m, 2 H) 3.55 (br.s., 1 H) 3.92 (s, 3 H) 6.95 (br.s., 1 H) 8.16 (dd, 1 H) 8.22- 8.25 (m, 2 H) 8.26-8.34 (m, 1 H) 8.40-8.46 (m, 1 H) 8.64 (br.s., 1 H) 8.78-8.85 (m, 1 H) 9.12 (d, 1 H) 9.43 (s, 1 H) |
| 80 | 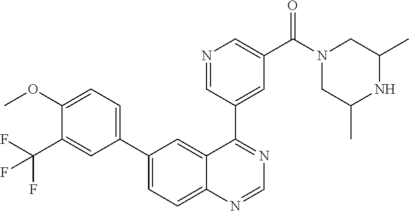 (3,5-Dimethyl-piperazin-1-yl)-{5-[6-(4-methoxy- 3-trifluoromethyl-phenyl)-quinazolin-4-yl]- pyridin-3-yl}-methanone | 0.99 ⁽¹⁾ | 522.3 | ¹H-NMR (400 MHz, DMSO-d₆, 298 K): δ ppm 0.82 (br.s., 3 H) 1.03 (br.s., 3 H) 2.34 (br.s., 1 H) 2.73 (br.s., 3 H) 3.52 (br.s., 1 H) 3.95 (s, 3 H) 4.41 (br.s., 1 H) 7.40 (d, 1 H) 7.98 (br.s., 1 H) 8.06 (d, 1 H) 8.21-8.23 (m, 2 H) 8.31 (t, 1 H) 8.43 (dd, 1 H) 8.84 (d, 1 H) 9.14 (d, 1 H) 9.43 (s, 1 H) |
| 81 | 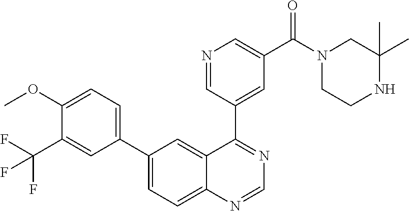 (3,3-Dimethyl-piperazin-1-yl)-{5-[6-(4-methoxy- 3-trifluoromethyl-phenyl)-quinazolin-4-yl]- pyridin-3-yl}-methanone | 0.99 ⁽¹⁾ | 522.3 | ¹H-NMR (400 MHz, DMSO-d₆, 298 K): δ ppm 0.90 (br.s., 3 H) 1.08 (br.s., 3 H) 2.69 (br.s., 1 H) 2.80 (br.s., 1 H) 3.17 (br.s., 1 H) 3.39 (m, 2 H) 3.56 (br.s., 1 H) 3.96 (s, 3 H) 7.40 (d, 1 H) 7.98 (br.s., 1 H) 8.07 (d, 1 H) 8.22 (d, 2 H) 8.30 (d, 1 H) 8.43 (m, 1 H) 8.83 (d, 1 H) 9.14 (d, 1 H) 9.42 (s, 1 H) |
| 82 | 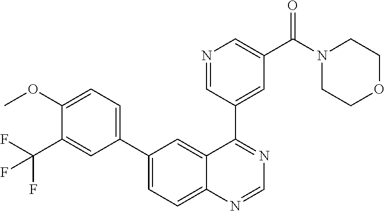 [SMILES: COc1ccc(-c2ccc3ncnc(-c4cncc(C(=O)N5CCOCC5)c4)c3c2)cc1C(F)(F)F] {5-[6-(4-Methoxy-3-trifluoromethyl-phenyl)- quinazolin-4-yl]-pyridin-3-yl}-morpholin-4-yl- methanone | 1.19 ⁽¹⁾ | 495.2 | ¹H-NMR (400 MHz, DMSO-d₆, 298 K): δ ppm 3.43-3.73 (m, 8 H) 3.95 (s, 3 H) 7.41 (d, 1 H) 8.00 (br.s., 1 H) 8.07 (d, 1 H) 8.21-8.25 (m, 2 H) 8.36 (t, 1 H) 8.44 (dd, 1 H) 8.87 (d, 1 H) 9.15 (d, 1 H) 9.42 (s, 1 H) |
| 83 | 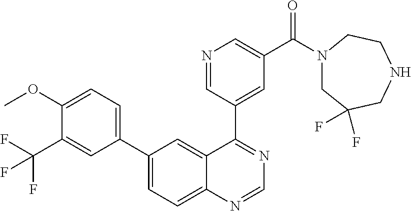 [SMILES: COc1ccc(-c2ccc3ncnc(-c4cncc(C(=O)N5CCNCC(F)(F)C5)c4)c3c2)cc1C(F)(F)F] (6,6-Difluoro-[1,4]diazepan-1-yl)-{5-[6-(4- methoxy-3-trifluoromethyl-phenyl)-quinazolin-4- yl]-pyridin-3-yl}-methanone | 1.01 ⁽¹⁾ | 544.2 | ¹H-NMR (400 MHz, DMSO-d₆, 298 K): δ ppm 2.81 (br.s., 1 H) 2.95-3.23 (m, 3 H) 3.49 (br.s., 1 H) 3.72-3.96 (m, 5 H) 4.16 (m, 1 H) 7.41 (d, 1 H) 7.97 (m, 1 H) 8.06 (m, 1 H) 8.18-8.24 (m, 2 H) 8.40 (br.s., 1 H) 8.45 (m, 1 H) 8.88 (m, 1 H) 9.15 (br.s., 1 H) 9.43 (s, 1 H) |

⁽¹⁾ LC methode 1,
⁽²⁾ LC methode 2

### Example 84

### {5-[6-(4-Methoxy-3-trifluoromethyl-phenyl)-quinazolin-4-yl]-pyridin-3-yl}-((S)-2-methyl-piperazin-1-yl)-methanone

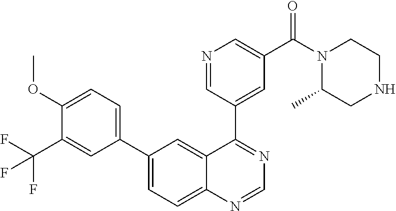 [SMILES: COc1ccc(-c2ccc3ncnc(-c4cncc(C(=O)N5CCNC[C@@H]5C)c4)c3c2)cc1C(F)(F)F]

To a solution of 5-[6-(4-methoxy-3-trifluoromethyl-phenyl)-quinazolin-4-yl)-nicotinic acid (70 mg, 0.165 mmol) in 3 mL of CH₂Cl₂ was added HBTU (68.7 mg, 0.181 mmol) and DIPEA (0.057 mL, 0.329 mmol). The reaction mixture was stirred at rt for 20 min, (S)-3-methyl-piperazine-1-carboxylic acid tert-butyl ester (49.4 mg, 0.247 mmol) and DIPEA (0.057 mL, 0.329 mmol) were added and the reaction mixture was stirred at ambient temperature for another 1 h. The reaction mixture was concentrated. The residue was dissolved in 2 ml of CH₂Cl₂ and TFA (0.120 mL, 1.646 mmol) was added. The reaction mixture was stirred at room temperature for 3 h. After this period of time, the mixture was concentrated and purified by preparative reverse phase Gilson HPLC and subsequent neutralization of the combined fractions over PL-HCO3 MP gave the title compound (60 mg, 68% yield) as a white powder. ¹H-NMR (400 MHz, DMSO-d₆, 298 K): δ ppm 1.24 (br.s., 3H) 2.53-2.89 (m, 7H) 3.96 (s, 3H) 7.41 (d, 1H) 7.99 (d, 1H) 8.07 (dd, 1H) 8.21-8.23 (dd, 2H) 8.30 (t, 1H), 8.44 (dd, 1H) 8.82 (d, 1H) 9.13 (d, 1H) 9.43 (s, 1H). MS: 508.3 [M+1]⁺, Rt⁽¹⁾=0.99 min.

Examples 85 was prepared using procedures analogous to those used for example 84, using appropriate starting materials.

| | | | MS (ES): | |
|---|---|---|---|---|
| Example | Structure/Name | Rt (min.) | [M + H]⁺ | 1H-NMR |
| 85 | 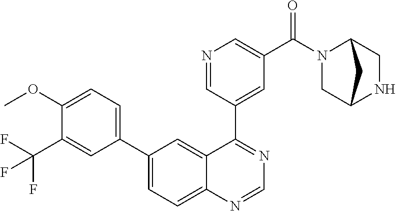 [SMILES: COc1ccc(-c2ccc3ncnc(-c4cncc(C(=O)N5C[C@@H]6C[C@H]5CN6)c4)c3c2)cc1C(F)(F)F] (1S,4S)-2,5-Diaza-bicyclo[2.2.1]hept-2-yl-{5-[6- (4-methoxy-3-trifluoromethyl-phenyl)- quinazolin-4-yl]-pyridin-3-yl}-methanone | 0.97 ⁽¹⁾ | 506.2 | ¹H-NMR (400 MHz, DMSO-d₆, 298 K): δ ppm 1.48-1.83 (m, 2 H) 2.62-3.07 (m, 2 H) 3.47-3.71 (m, 3 H) 3.96 (s, 3 H) 4.39-4.71 (d, 1 H) 7.39-7.44 (m, 1 H) 7.99 (d, 1 H) 8.8 (m, 1 H) 8.19- 8.28 (m, 2 H) 8.33-8.43 (dt, 1 H) 8.45 (d, 1 H) 8.93 (dd, 1 H) 9.16 (dd, 1 H) 9.43 (s, 1 H) |

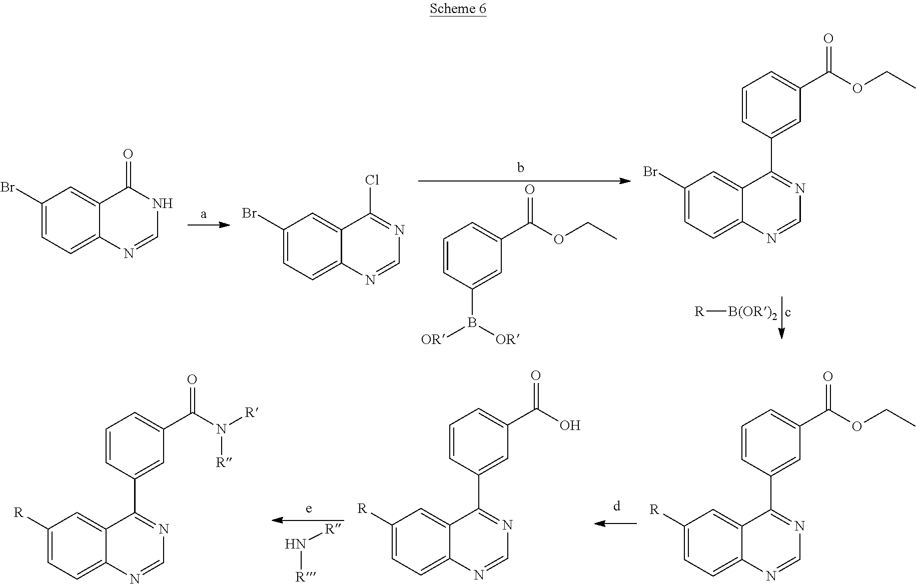

a) Choronation of 6-Bromo-3H-quinazolin-4-one is performed under customary phophorus oxychoride condition by heating at reflux or 130° C. in diluted (such as in CH₂Cl₂) or neat phophorus oxychoride. b) Suzuki cross-coupling between 6-Bromo-4-chloro-quinazoline and 3-(ethoxycarbonyl)phenyl-boronic acid or 3-(ethoxycarbonyl)phenyl-boronate is performed under customary Suzuki conditions using palladium catalyst such as preferably Dichlorodiphenylphosphine palladium (PdCl₂(PPh₃)₂), aqueous base and organic solvent such as preferably acetonitrile. The reaction is preferably stirred at a temperature of approximately 100-120° C. in preferably a microwaves oven. The reaction may preferably carried out under an inert gas such as nitrogen or atrgon. c) Suzuki cross-coupling between aryl bromide and boronic acid or boronic acid derivatives such as boronate of formula R—B(OR′)₂ is performed under customary Suzuki conditions using palladium catalyst such as preferably palladium tetrakis(triphenylphosphine) palladium (Pd(PPh₃)₄), aqueous base and organic solvent such as preferably acetonitrile. The reaction is preferably stirred at a temperature of approximately 100-120° C. in preferably a microwaves oven. The reaction may preferably carried out under an inert gas such as nitrogen or argon. d) Saponification of the carboxylic ester was performed under customary saponification conditions, using among the possible aqueous bases lithium hydroxyide is preferred and organic solvent such a preferably dioxane. The reation may preferably be carried out at room temperature. e) Condenation of a carboxylic acid with amines of the formula R″′NHR″ preferably takes place under customary condensation conditions. The reaction can be carried on by dissolving the carboxylic acid and the amine of formula R″′NHR″ in a suitable solvent, for example halogenated hydrocarbon, such as methylene chloride, N,N-dimethylformamide, N,Ndimethylacetamide, N-2-methyl-pyrrolidone, methylene chloride, or a mixture of two or more such solvents, and by the addition of a suitable base, for example triethylamine, diisopropylethylamine (DIPEA) or N-methylmorpholine and a suitable coupling agent that forms a reactive derivative of the carboxylic acid in situ, for example and preferably (2-(1H-Benzotriazole-1-yl)-1,1,3,3-tetramethyluronium hexafluorophosphate (HBTU). The reaction mixture is preferably stirred at a temperature of from approximately −20 to 50° C., especially from −5° C. to 30° C., e.g at 0° C. to room temperature. The reaction my preferably be carried out under an inert gas, e.g. nitrogenor argon.

The final compounds described herein were according the general procedure described in scheme 6.

### Example 86

### (4-Ethyl-piperazin-1-yl)-{3-[6-(6-methoxy-pyridin-3-yl)-quinazolin-4-yl]-phenyl}-methanone

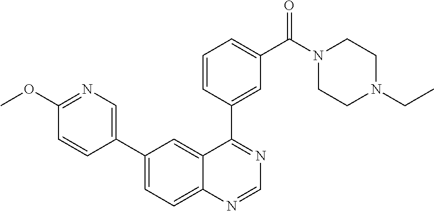 [SMILES: CCN1CCN(C(=O)c2cccc(-c3ncnc4ccc(-c5ccc(OC)nc5)cc34)c2)CC1]

To a stirred solution of 3-[6-(6-methoxy-pyridin-3-yl)-quinazolin-4-yl]-benzoic acid (100 mg, 0.280 mmol) in 2 mL of CH₂Cl₂, was added HBTU (127 mg, 0.336 mmol) and DIPEA (0.147 mL, 0.839 mmol). The reaction mixture was stirred at rt for 10 min, 1-ethyl-piperazine (38 mg, 0.336 mmol) was added and the resulting reaction mixture stirred for a further 30 min at rt. The reaction was quenched with H₂O, and extracted with CH₂Cl₂. The organic layer was washed with brine, dried over MgSO₄, filtered and evaporated under vacuum. Purification by preparative reverse phase Gilson HPLC and subsequent neutralization of the combined fractions over PL-HCO₃ MP gave the title compound (40 mg, 35% yield) as a white powder. ¹H-NMR (400 MHz, DMSO-d₆, 298 K): δ ppm 0.97 (t, 3H) 2.18-2.50 (m, 6H) 3.37-3.71 (m, 4 H) 3.91 (s, 3H) 6.97 (d, 1H) 7.66 (d, 1H) 7.74 (dd, 1H) 7.83 (s, 1H) 7.99 (d, 1H) 8.12 (d, 1 H) 8.23 (br.s, 2H) 8.38 (d, 1H) 8.60 (s, 1H) 9.39 (s, 1H). MS: 454.2 [M+1]⁺, Rt⁽²⁾=0.89 min.

### 3-[6-(6-Methoxy-pyridin-3-yl)-quinazolin-4-yl]-benzoic acid

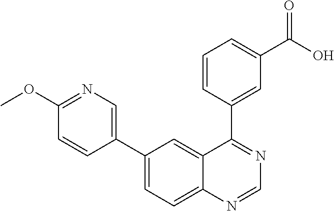 [SMILES: COc1ccc(-c2ccc3ncnc(-c4cccc(C(=O)O)c4)c3c2)cn1]

To a suspension of 3-[6-(6-methoxy-pyridin-3-yl)-quinazolin-4-yl]-benzoic acid ethyl ester (800 mg, 1.91 mmol) in dioxane (20 mL) was added at rt a 1M aqueous solution of LiOH.H₂O (9.55 ml, 9.55 mmol) and the reaction mixture was stirred 4 h at rt. The reaction was quenched with a 1M aqueous solution of HCl (5 mL), the formed precipitate was filtered and dried under vacuum to gave the title compound (700 mg, 90% purity, 92% yield) as a light yellow solid. The compound was used in the next step without further purification. MS: 358.1 [M+1]⁺, Rt⁽²⁾=1.11 min.

### 3-[6-(6-Methoxy-pyridin-3-yl)-quinazolin-4-yl]-benzoic acid ethyl ester

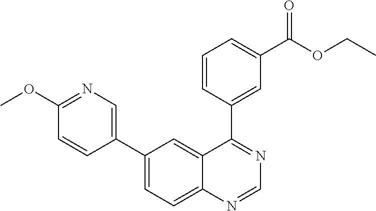 [SMILES: CCOC(=O)c1cccc(-c2ncnc3ccc(-c4ccc(OC)nc4)cc23)c1]

To a mixture of 3-(6-bromo-quinazolin-4-yl)-benzoic acid ethyl ester (845 mg, 2.176 mmol), 2-methoxy-5-pyridineboronic acid (399 mg, 2.61 mmol) and Pd(PPh₃)₄ (126 mg, 0.109 mmol) was added 20 mL of DME. The reaction mixture was flushed with argon and a 1M aqueous solution of Na₂CO₃ (4.35 mL, 4.35 mmol) was added and the vial capped. The reaction mixture was heated to 120° C. for 15 min using a microwave oven then cooled down to rt, diluted with CH₂Cl₂, filtered through a Celite pad and portioned between brine/CH₂Cl₂. The organic layer was washed with brine, dried over MgSO₄, filtered and evaporated. Purification by flash chromatography on silica gel (CH₂Cl₂/MeOH, 95/5) gave the title compound (800 mg, 92% purity, 88% yield). MS: 386.5 [M+1]⁺, Rt⁽²⁾=1.45 min.

Examples 87 to 96, were prepared using procedures analogous to those used for example 86, using appropriate starting materials.

| | | | MS (ES): | |
|---|---|---|---|---|
| Example | Structure/Name | Rt (min.) | [M + H]⁺ | 1H-NMR |
| 87 | 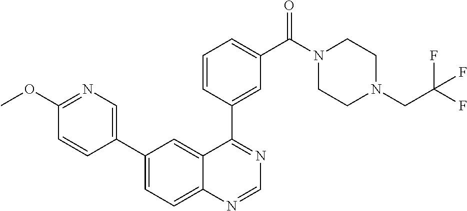 [SMILES: COc1ccc(-c2ccc3ncnc(-c4cccc(C(=O)N5CCN(CC(F)(F)F)CC5)c4)c3c2)cn1] {3-[6-(6-Methoxy-pyridin-3-yl)-quinazolin-4-yl]- phenyl}-[4-(2,2,2-trifluoro-ethyl)-piperazin-1-yl]- methanone | 1.36 ⁽¹⁾ | 508.3 | ¹H-NMR (400 MHz, DMSO-d₆, 298 K): δ ppm 2.46 (br.s., 2 H) 2.68 (br.s., 2 H) 3.17 (q, 2 H) 3.45 (br.s., 2 H) 3.64 (br.s., 2 H) 3.91 (s, 3 H) 6.96 (d, 1 H) 7.66 (dt, 1 H) 7.74 (t, 1 H) 7.85 (br.s., 1 H) 8.00 (dt, 1 H) 8.12 (dd, 1 H) 8.21 (d, 1 H) 8.24 (d, 1 H) 8.40 (dd, 1 H) 8.60 (d, 1 H) 9.39 (s, 1 H) |
| 88 | 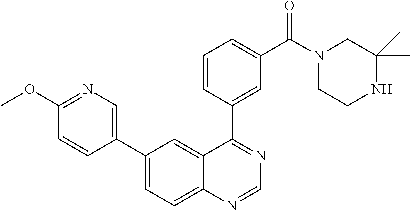 (3,3-Dimethyl-piperazin-1-yl)-{3-[6-(6-methoxy- pyridin-3-yl)-quinazolin-4-yl]-phenyl}- methanone | 0.95 ⁽²⁾ | 454.3 | ¹H-NMR (400 MHz, DMSO-d₆, 298 K): δ ppm 0.92 (br.s., 3 H) 1.08 (br.s., 3 H) 2.65-2.85 (m, 2 H) 3.17 (br.s., 1 H) 3.35-3.40 (m, 2 H) 3.57 (br.s., 1 H) 3.91 (s, 3 H) 6.96 (d, 1 H) 7.64 (br.s., 1 H) 7.73 (t, 1 H) 7.83 (br.s., 1 H) 7.98 (d, 1 H) 8.12 (dd, 1 H) 8.20-8.22 (m, 2 H) 8.39 (d, 1 H) 8.59 (d, 1 H) 9.38 (s, 1 H) |
| 89 | 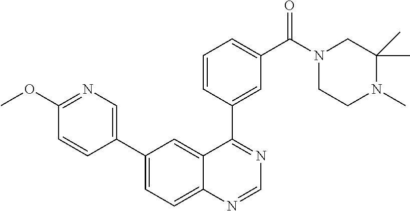 {3-[6-(6-Methoxy-pyridin-3-yl)-quinazolin-4-yl]- phenyl}-(3,3,4-trimethyl-piperazin-1-yl)- methanone | 0.95 ⁽²⁾ | 468.3 | ¹H-NMR (400 MHz, DMSO-d₆, 298 K): δ ppm 0.82 (br.s., 3 H) 0.97 (br.s., 3 H) 2.09 (br.s., 3 H) 2.33 (br.s., 1 H) 3.16 (br.s., 1 H) 3.35-3.50 (m, 3 H) 3.65 (br.s., 1 H) 3.91 (s, 3 H) 6.95 (d, 1 H) 7.64 (br.s., 1 H) 7.73 (t, 1 H) 7.83 (br.s., 1 H) 7.99 (d, 1 H) 8.11 (dd, 1 H) 8.19-8.23 (m, 2 H) 8.38 (dd, 1 H) 8.58 (d, 1 H) 9.38 (s, 1 H) |
| 90 | 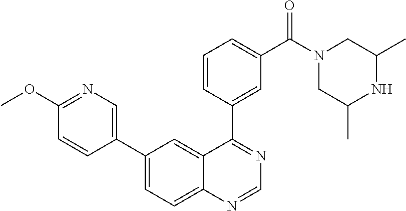 (3,5-Dimethyl-piperazin-1-yl)-{3-[6-(6-methoxy- pyridin-3-yl)-quinazolin-4-yl]-phenyl}- methanone | 0.94 ⁽²⁾ | 454.2 | ¹H-NMR (400 MHz, DMSO-d6, 298 K): δ ppm 0.83 (br.s., 3 H) 1.02 (br.s., 3 H) 2.29 (br.s., 1 H) 2.68 (br.s., 3 H) 3.51 (br.s., 1 H) 3.91 (s, 3 H) 4.40 (br.s., 1 H) 6.96 (d, 1 H) 7.64 (dt, 1 H) 7.72 (t, 1 H) 7.84 (br.s., 1 H) 7.98 (dt, 1 H) 8.11 (dd, 1 H) 8.20- 8.23 (m, 2 H) 8.39 (d, 1 H) 8.59 (d, 1 H) 9.38 (s, 1 H) |
| 91 | 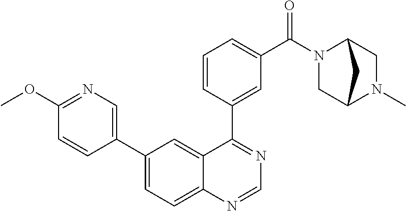 {3-[6-(6-Methoxy-pyridin-3-yl)-quinazolin-4-yl]- phenyl}-((1S,4S)-5-methyl-2,5-diaza- bicyclo[2.2.1]hept-2-yl)-methanone | 0.92 ⁽²⁾ | 452.2 | ¹H-NMR (400 MHz, DMSO-d₆, 298 K): δ ppm 1.69 (s, 1 H) 1.72- 1.79 (dd, 1 H) 2.26 (d, 3 H) 2.50-2.83 (m, 2 H) 3.33-3.40 (m, 1 H) 3.41-3.52 (m, 2 H) 3.91 (s, 3 H) 4.28-4.61 (d, 1 H) 6.96 (d, 1 H) 7.70-7.82 (m, 2 H) 7.89-8.05 (m, 2 H) 8.09-8.16 (m, 1 H) 8.19- 8.26 (m, 2 H) 8.37-8.42 (d, 1 H) 8.57-8.63 (m, 1 H) 9.39 (s, 1 H) |
| 92 | 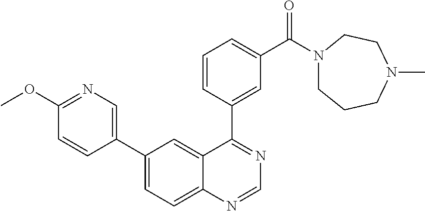 {3-[6-(6-Methoxy-pyridin-3-yl)-quinazolin-4-yl]- phenyl}-(4-methyl-[1,4]diazepan-1-yl)- methanone | 0.93 ⁽²⁾ | 454.2 | ¹H-NMR (400 MHz, DMSO-d₆, 298 K): δ ppm 1.74 (m, 1 H) 1.85 (m, 1 H) 2.15- 2.29 (br.s., 3 H) 2.40-2.66 (m, 4 H) 3.45-3.54 (m, 2 H) 3.61-3.69 (m, 2 H) 3.91 (s, 3 H) 6.96 (d, 1 H) 7.65 (d, 1 H) 7.72 (t, 1 H) 7.83 (br.s., 1 H) 7.98 (m, 1 H) 8.12 (dt, 1 H) 8.21 (d, 2 H) 8.39 (dd, 1 H) 8.59 (br.s., 1 H) 9.38 (s, 1 H) |
| 93 | 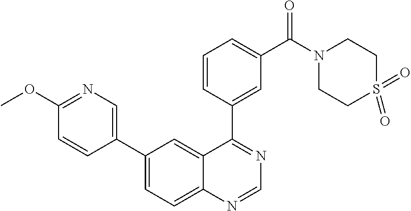 [SMILES: COc1ccc(-c2ccc3ncnc(-c4cccc(C(=O)N5CCS(=O)(=O)CC5)c4)c3c2)cn1] (1,1-Dioxo-1lambda*6*-thiomorpholin-4-yl)-{3- [6-(6-methoxy-pyridin-3-yl)-quinazolin-4-yl]- phenyl}-methanone | 1.02 ⁽¹⁾ | 475.2 | ¹H-NMR (400 MHz, DMSO-d₆, 298 K): δ ppm 3.07-3.38 (br.s., 4 H) 3.67-3.89 (br.s., 2 H) 3.91 (s, 3 H) 3.94-4.17 (br.s., 2 H) 6.97 (d, 1 H) 7.74 (d, 2 H) 8.02 (m, 2 H) 8.13 (dd, 1 H) 8.22 (d, 1 H) 8.24 (d, 1 H) 8.40 (dd, 1 H) 8.60 (d, 1 H) 9.39 (s, 1 H) |
| 94 | 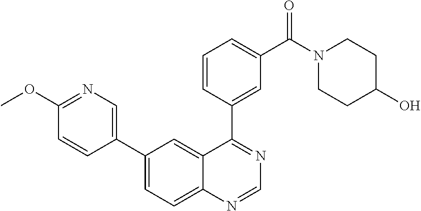 [SMILES: COc1ccc(-c2ccc3ncnc(-c4cccc(C(=O)N5CCC(O)CC5)c4)c3c2)cn1] (4-Hydroxy-piperidin-1-yl)-{3-[6-(6-methoxy- pyridin-3-yl)-quinazolin-4-yl]-phenyl}- methanone | 0.97 ⁽²⁾ | 441.5 | ¹H-NMR (400 MHz, DMSO-d₆, 298 K): δ ppm 1.25-1.47 (m, 2 H) 1.62-1.86 (m, 2 H) 3.23 (m, 2 H) 3.54-3.68 (m, 1 H) 3.69-3.76 (m, 1 H) 3.92 (s, 3 H) 3.97-4.11 (m, 1 H) 4.78 (d, 1 H) 6.96 (d, 1 H) 7.64 (d, 1 H) 7.72 (t, 1 H) 7.85 (s, 1 H) 7.98 (dt, 1 H) 8.12 (dd, 1 H) 8.20- 8.23 (m, 2 H) 8.40 (d, 1 H) 8.60 (s, 1 H) 9.38 (s, 1 H) |
| 95 | 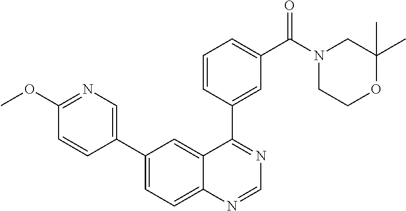 (2,2-Dimethyl-morpholin-4-yl)-{3-[6-(6-methoxy- pyridin-3-yl)-quinazolin-4-yl]-phenyl}- methanone | 1.22 ⁽²⁾ | 455.7 | ¹H-NMR (400 MHz, DMSO-d₆, 298 K): δ ppm 1.06 (br.s., 3 H) 1.18 (br.s., 3 H) 3.37-3.73 (m, 6 H) 3.91 (s, 3 H) 6.95 (d, 1 H) 7.67 (br.s., 1 H) 7.74 (t, 1 H) 7.87 (br.s., 1 H) 8.00 (d, 1 H) 8.12 (dd, 1 H) 8.21 (d, 1 H) 8.24 (d, 1 H) 8.39 (dd, 1 H) 8.59 (d, 1 H) 9.39 (s, 1 H) |
| 96 | 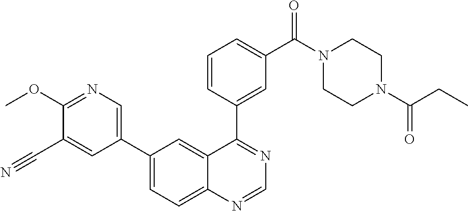 [SMILES: CCC(=O)N1CCN(C(=O)c2cccc(-c3ncnc4ccc(-c5cnc(OC)c(C#N)c5)cc34)c2)CC1] 2-Methoxy-5-{4-[3-(4-propionyl-piperazine-1- carbonyl)-phenyl]-quinazolin-6-yl}-nicotinonitrile | 1.09 ⁽²⁾ | 507.6 | ¹H-NMR (400 MHz, DMSO-d₆, 298 K): δ ppm 0.98 (t, 3 H) 2.17- 2.40 (m, 2 H) 3.35-3.72 (m, 8 H) 4.07 (s, 3 H) 7.70 (d, 1 H) 7.75 (t, 1 H) 7.90 (br.s., 1 H) 8.02 (d, 1 H) 8.23 (d, 1 H) 8.35 (d, 1 H) 8.43 (dd, 1 H) 8.80 (br.s., 1 H) 8.91 (br.s., 1 H) 9.41 (s, 1 H) |

⁽¹⁾ LC methode 1,
⁽²⁾ LC methode 2

### Example 97

### {3-[6-(6-Methoxy-pyridin-3-yl)-quinazolin-4-yl]-phenyl}-((R)-2-methyl-piperazin-1-yl)-methanone

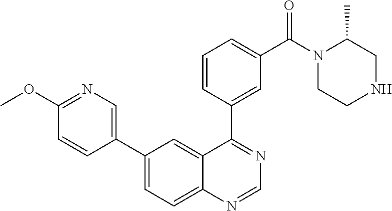 [SMILES: COc1ccc(-c2ccc3ncnc(-c4cccc(C(=O)N5CCNC[C@H]5C)c4)c3c2)cn1]

To a stirred solution of 3-[6-(6-methoxy-pyridin-3-yl)-quinazolin-4-yl]-benzoic acid (100 mg, 0.254 mmol) in 2 mL of DMF, was added HBTU (144 mg, 0.381 mmol) and DIPEA (0.177 mL, 1.016 mmol). The reaction mixture was stirred at rt for 30 min, (R)-3-methyl-piperazine-1-carboxylic acid tert-butyl ester (76 mg, 0.381 mmol) was added and the resulting reaction mixture stirred for a further 2 h at rt. The reaction was quenched with H₂O, and extracted with CH₂Cl₂. The organic layer was washed with brine, dried over MgSO₄, filtered and evaporated under vacuum. The residue was dissolved in 3 ml of CH₂Cl₂ and TFA (1 ml) was added. The reaction mixture was stirred at ambient temperature for 2 h. After this period of time, the mixture was concentrated and purified by preparative reverse phase Gilson HPLC and subsequent neutralization of the combined fractions over PL-HCO₃ MP gave the title compound (29 mg, 26% yield) as a white powder. ¹H-NMR (400 MHz, DMSO-d₆, 298 K): δ ppm 1.23 (d, 3H) 2.55-3.2 (m, 7H) 3.91 (s, 3H) 6.95 (d, 1H) 7.62 (d, 1H) 7.72 (t, 1H) 7.81 (s, 1H) 7.98 (d, 1H) 8.11 (d, 1H) 8.22 (d, 2H) 8.38 (d, 1H) 8.59 (s, 1H) 9.38 (s, 1H). MS: 440.1 [M+1]⁺, Rt⁽²⁾=0.89 min.

### Example 98

### {3-[6-(6-Methoxy-pyridin-3-yl)-quinazolin-4-yl]-phenyl}-((S)-2-methyl-piperazin-1-yl)-methanone

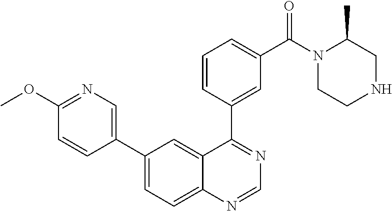 [SMILES: COc1ccc(-c2ccc3ncnc(-c4cccc(C(=O)N5CCNC[C@@H]5C)c4)c3c2)cn1]

To a stirred solution of 3-[6-(6-methoxy-pyridin-3-yl)-quinazolin-4-yl]-benzoic acid (110 mg, 0.308 mmol) in 2.5 mL of DMF, was added HBTU (175 mg, 0.462 mmol) and DIPEA (0.108 mL, 0.616 mmol). The reaction mixture was stirred at rt for 20 min, (S)-3-Methyl-piperazine-1-carboxylic acid tert-butyl ester (123 mg, 0.616 mmol) and DIPEA (0.108 mL, 0.616 mmol) were added and the resulting reaction mixture stirred for a further 2 h at rt. The reaction was quenched with H₂O, and extracted with CH₂Cl₂. The organic layer was washed with brine, dried over MgSO₄, filtered and evaporated under vacuum. Purification by flash chromatography on silica gel (Heptane/Ethylacetate, 1/1) gave the intermediate compound (170 mg, 91% purity (UPLC), 93% yield), MS: 540.3 [M+1]⁺. This residue (170 mg, 0.287 mmol) was dissolved in 2 ml of CH₂Cl₂ and TFA (0.331 mL, 4.30 mmol) was added. The reaction mixture was stirred at ambient temperature for 2 h. After this period of time, the mixture was quenched with a solution of NaOH (1M) and extracted with CH₂Cl₂. The organic layer was washed with brine, dried over MgSO₄, filtered and evaporated under vacuum. Purification by preparative reverse phase Gilson HPLC and subsequent neutralization of the combined fractions over PL-HCO₃ MP gave the title compound (58 mg, 31% yield) as a white powder. ¹H-NMR (400 MHz, DMSO-d₆, 298 K): δ ppm 1.23 (d, 3H) 2.55-3.15 (m, 7H) 3.91 (s, 3H) 6.95 (d, 1H) 7.62 (d, 1H) 7.72 (t, 1H) 7.81 (s, 1H) 7.98 (dt, 1H) 8.12 (dd, 1H) 8.21 (d, 1H) 8.22 (s, 1H) 8.38 (dd, 1H) 8.59 (d, 1H) 9.38 (s, 1H). MS: 440.3 [M+1]⁺, Rt⁽¹⁾=0.85 min.

### Example 99

### ((S)-2,4-Dimethyl-piperazin-1-yl)-{3-[6-(6-methoxy-pyridin-3-yl)-quinazolin-4-yl]-phenyl}-methanone

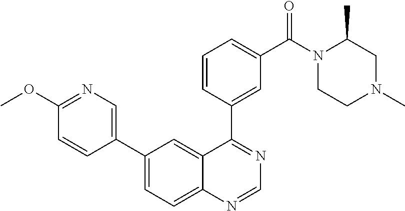

To a solution of {3-[6-(6-methoxy-pyridin-3-yl)-quinazolin-4-yl]-phenyl}-((S)-2-methyl-piperazin-1-yl)-methanone (50 mg, 0.091 mmol) in 1 mL of MeOH was added a 37% solution of formaldehyde (0.008 mL, 0.109 mmol). The reaction mixture was stirred at rt for 30 min, then NaBH₃CN (6.86 mg, 0.109 mmol) was added and the resulting reaction mixture stirred for a further 2 h at rt. The reaction was quenched with a solution of NaHCO₃ sat and extracted with ethylacetate. The organic layer was washed with brine, dried over MgSO₄, filtered and evaporated under vacuum. Purification by preparative reverse phase Gilson HPLC and subsequent neutralization of the combined fractions over PL-HCO₃ MP gave the title compound (20 mg, 48% yield) as a white powder. ¹H-NMR (400 MHz, DMSO-d₆, 298 K): δ ppm 1.25 (d, 3H) 1.82 (m, 1H) 1.99 (m, 1H) 2.11 (s, 3H) 2.53-2.74 (m, 3H) 3.12-3.27 (m, 2H) 3.91 (s, 3H) 6.96 (d, 1H) 7.63 (dt, 1H) 7.74 (t, 1H) 7.81 (br.s., 1H) 7.99 (dt, 1H) 8.11 (dd, 1H) 8.21 (d, 1H) 8.22 (s, 1H) 8.39 (dd, 1H) 8.59 (d, 1H) 9.39 (s, 1H). MS: 454.3 [M+1]⁺, Rt⁽¹⁾=0.85 min.

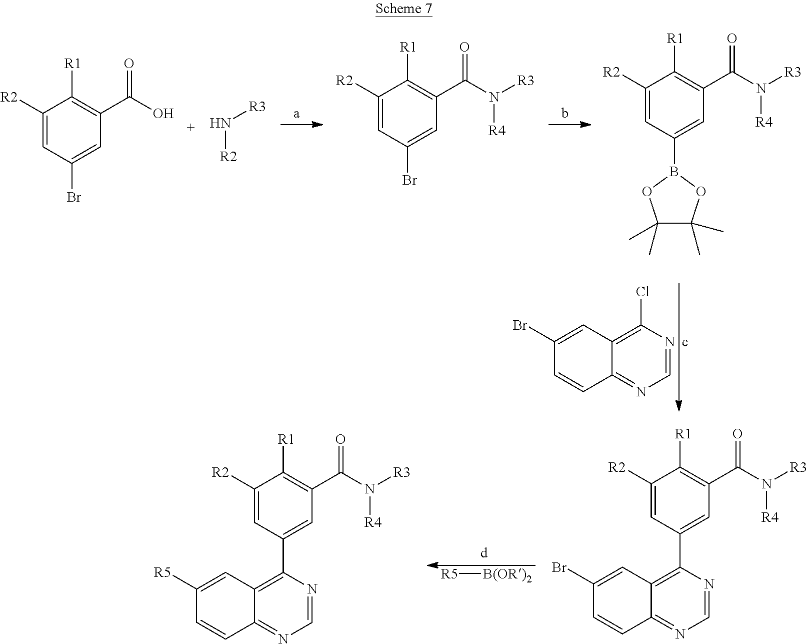

a) Condenation of a carboxylic acid with amines of the formula R3NHR4 preferably takes place under customary condensation conditions. The reaction can be carried on by dissolving the carboxylic acid and the amine of formula R3NHR4 in a suitable solvent, for example halogenated hydrocarbon, such as methylene chloride, N,N-dimethylformamide, N,N-dimethylacetamide, N-2-methyl-pyrrolidone, methylene chloride, or a mixture of two or more such solvents, and by the addition of a suitable base, for example triethylamine, diisopropylethylamine (DIPEA) or N-methylmorpholine and a suitable coupling agent that forms a reactive derivative of the carboxylic acid in situ, for example and preferably (2-(1 H-Benzotriazole-1-yl)-1,1,3,3-tetramethyluronium hexafluorophosphate (HBTU). The reaction mixture is preferably stirred at a temperature of from approximately −20 to 50° C., especially from −5° C. to 30° C., e.g at 0° C. to room temperature. The reaction my preferably be carried out under an inert gas, e.g. nitrogenor argon. b) Formation of the boronate ester was performed using palladium catalyst such as preferably 1,1-Bis(diphenylphosphino) -ferrocene[dichloropalladium (PdCl2(dppf)-CH₂Cl₂), aqueous base such as preferably potassium acetate organic solvent such as preferably dioxane and Bis-(pinacolato)-diboron. The reaction is preferably stirred at approximately 80° C. for several hours. c) Suzuki cross-coupling between 6-Bromo-4-chloro-quinazoline and the boronate is performed under customary Suzuki conditions using Dichlorodiphenylphosphine palladium (PdCl₂(PPh₃)₂), aqueous base and organic solvent such as preferably acetonitrile. The reaction is preferably stirred at a temperature of approximately 100-120° C. in preferably a microwaves oven. The reaction may preferably carried out under an inert gas such as nitrogen or atrgon. d) Suzuki cross-coupling between aryl bromide and boronic acid or boronic acid derivatives such as boronate of formula R5-B(OR′)₂ is performed under customary Suzuki conditions using palladium catalyst such as preferably palladium tetrakis(triphenylphosphine) palladium (Pd(PPh₃)₄), aqueous base and organic solvent such as preferably acetonitrile. The reaction is preferably stirred at a temperature of approximately 100-120° C. in preferably a microwaves oven. The reaction may preferably carried out under an inert gas such as nitrogen or argon

The final compounds described herein were according the general procedure described in scheme 7.

### Example 100

### 1-(4-{5-[6-(6-Methoxy-pyridin-3-yl)-quinazolin-4-yl]-2-methyl-benzoyl}-piperazin-1-yl)-ethanone

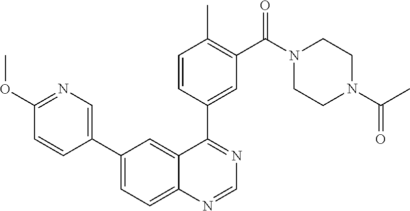 [SMILES: COc1ccc(-c2ccc3ncnc(-c4ccc(C)c(C(=O)N5CCN(C(C)=O)CC5)c4)c3c2)cn1]

A mixture of 1-{4-[5-(6-bromo-quinazolin-4-yl)-2-methyl-benzoyl]-piperazin-1-yl}-ethanone (150 mg, 0.331 mmol), 6-methoxypyridin-3-ylboronic acid (50.6 mg, 0.331 mmol), K₃PO₄ (105 mg, 0.496 mmol) and PdCl₂(PPh₃)₂ (11.61 mg, 0.017 mmol) was flushed with argon for few minutes. To the mixture was then added 4 ml of Acetonitrile followed by 0.4 ml of water. The vial was capped and the reaction mixture was heated to 120° C. for 10 min using a microwave oven. The mixture was then cooled down to rt, diluted with CH₂Cl₂ and filtered through a Celite pad. The organic layer was washed with sat. Bicarbonate solution, dried by passing through a phase separating cartridge and evaporated. Purification by preparative reverse phase Gilson HPLC and subsequent neutralization of the combined fractions over SCx-2 cartridge gave the title compound (100 mg, 60% yield) as a powder. ¹H-NMR (400 MHz, DMSO-d₆) δ ppm 1.90-2.10 (m, 3H) 2.37 (s, 3H) 3.20-3.80 (br. m., 8H) 3.91 (s, 3H), 6.96 (dd, 1H) 7.57 (dd, 1H) 7.73 (d, 1H) 7.87 (dd, 1H), 8.12 (d, 1H) 8.18 (s, 1H) 8.20 (s, 1H) 8.23 (br. s., 1H) 8.38 (dd, 1H) 8.60 (br. s., 1H) 9.36 (s, 1H). MS: 482.3 [M+1]⁺, Rt⁽¹⁾=1.01 min.

### 1-{4-[5-(6-Bromo-quinazolin-4-yl)-2-methyl-benzoyl]-piperazin-1-yl}-ethanone

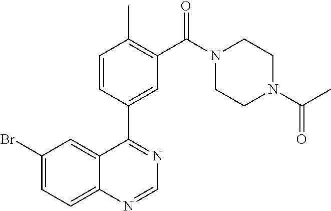 [SMILES: CC(=O)N1CCN(C(=O)c2cc(-c3ncnc4ccc(Br)cc34)ccc2C)CC1]

A mixture of 6-bromo-4-chloroquinazoline (1.8 g, 7.39 mmol) (commercial source), 1-{-4-[2-methyl-5-(4,4,5,5-tetramethyl-[1,3,2]dioxaborolan-2-yl)-benzoyl]-piperazin-1-yl}-ethanone (4.23 g, 7.39 mmol, 65% purity (UPLC)), K₃PO₄ (2.354 g, 11.09 mmol) and PdCl₂(PPh₃)₂ (0.259 g, 0.370 mmol) was flushed with argon for few minutes. To the mixture was then added 15 ml of Acetonitrile followed by 1.5 ml of water. The vial was capped and the reaction mixture was heated to 120° C. for 10 min using a microwave oven. The mixture was then cooled down to rt, diluted with CH₂Cl₂ and filtered through a Celite pad. The organic layer was washed with sat. Bicarbonate solution, dried by passing through a phase separating cartridge and evaporated. Purification by Flash chromatography using Biotage Isolera system (amine functionalized silica KP-NH, eluting with Cyclohexane/EtOAc 0 to 100%) gave the title compound (1.65 g, 49% yield) as a yellow powder. MS: 453.2-455.1 [M+1]⁺, Rt⁽¹)=0.99 min.

### Example 101

### 1-(4-{3-[6-(6-Methoxy-pyridin-3-yl)-quinazolin-4-yl]-5-trifluoromethyl-benzoyl}-piperazin-1-yl)-ethanone

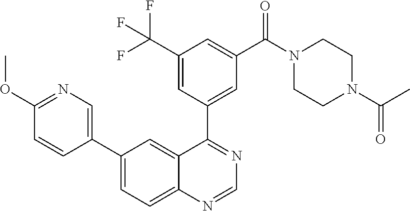 [SMILES: COc1ccc(-c2ccc3ncnc(-c4cc(C(=O)N5CCN(C(C)=O)CC5)cc(C(F)(F)F)c4)c3c2)cn1]

To a mixture of 1-{4-[3-(6-bromo-quinazolin-4-yl)-5-trifluoromethyl-benzoyl]-piperazin-1-yl}-ethanone (120 mg, 0.19 mmol, 80% purity (HPLC)), 2-methoxy-5-pyridine boronic acid (34.9 mg, 0.228 mmol) and Pd(PPh₃)₄ (10.98 mg, 0.009 mmol) was added 2 mL of Acetonitrile. The reaction mixture was flushed with argon and a 1M aqueous solution of Na₂CO₃ (0.380 mL, 0.380 mmol) was added and the vial capped. The reaction mixture was heated to 120° C. for 10 min using a microwave oven then cooled down to rt, diluted with EtOAc, filtered through a Celite pad and washed with EtOAc. The filtrate was concentrated. Purification by preparative reverse phase Gilson HPLC and subsequent neutralization of the combined fractions over PL-SO₃H MP gave the title compound (48 mg, 47% yield) as a white powder. ¹H-NMR (400 MHz, DMSO-d₆, 298 K): δ ppm 1.91-2.03 (br.s., 3H) 3.36-3.70 (m, 8H) 3.91 (s, 3H) 6.98 (d, 1H) 8.06 (br.s., 1H) 8.14 (d, 1H) 8.25 (d, 3H) 8.30 (br.s., 1H) 8.44 (d, 1H) 8.63 (br.s., 1H) 9.42 (s, 1H). MS: 536.6 [M+1]⁺, Rt⁽²⁾=1.18 min.

Examples 102 to 109, were prepared using procedures analogous to those used for example 101, using appropriate starting materials.

| | | | MS (ES): | |
|---|---|---|---|---|
| Example | Structure/Name | Rt (min.) | [M + H]⁺ | 1H-NMR |
| 102 | 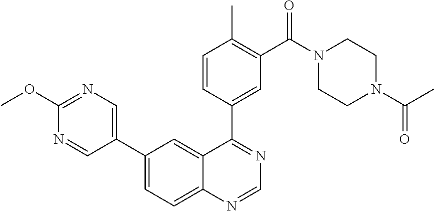 [SMILES: COc1ncc(-c2ccc3ncnc(-c4ccc(C)c(C(=O)N5CCN(C(C)=O)CC5)c4)c3c2)cn1] 1-(4-{5-[6-(2-Methoxy-pyrimidin-5-yl)- quinazolin-4-yl]-2-methyl-benzoyl}-piperazin-1- yl)-ethanone | 0.89 ⁽¹⁾ | 483.3 | ¹H-NMR (400 MHz, DMSO-d₆, 298 K): δ ppm 1.90-2.06 (m, 3 H) 2.37 (s, 3 H) 3.20-3.80 (br. m., 8 H) 3.98 (s, 3 H) 7.57 (d, 1 H) 7.75 (s, 1 H) 7.88 (d, 1H) 8.21 (d, 1 H) 8.32 (s, 1 H) 8.41 (d, 1 H) 9.04 (d, 2 H) 9.37 (s, 1 H) |
| 103 | 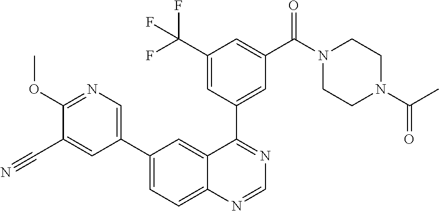 [SMILES: COc1ncc(-c2ccc3ncnc(-c4cc(C(=O)N5CCN(C(C)=O)CC5)cc(C(F)(F)F)c4)c3c2)cc1C#N] 5-{4-[3-(4-Acetyl-piperazine-1-carbonyl)-5- trifluoromethyl-phenyl]-quinazolin-6-yl}-2- methoxy-nicotinonitrile | 1.22 ⁽¹⁾ | 561.3 | ¹H-NMR (400 MHz, DMSO-d₆, 298 K): δ ppm 1.87-2.07 (br.s., 3 H) 3.34-3.74 (m, 8 H) 4.06 (s, 3 H) 8.07 (br.s., 1 H) 8.21 (br.s., 1 H) 8.23 (d, 1 H) 8.30 (br.s., 1 H) 8.35 (d, 1 H) 8.46 (d, 1 H) 8.79 (br.s., 1 H) 8.92 (br.s., 1 H) 9.43 (s, 1 H) |
| 104 | 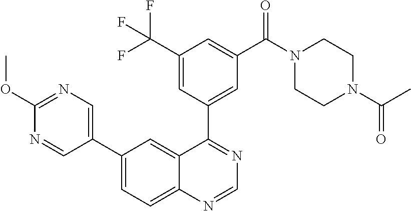 [SMILES: COc1ncc(-c2ccc3ncnc(-c4cc(C(=O)N5CCN(C(C)=O)CC5)cc(C(F)(F)F)c4)c3c2)cn1] 1-(4-{3-[6-(2-Methoxy-pyrimidin-5-yl)- quinazolin-4-yl]-5-trifluoromethyl-benzoyl}- piperazin-1-yl)-ethanone | 1.06 ⁽²⁾ | 537.6 | ¹H-NMR (400 MHz, DMSO-d₆, 298 K): δ ppm 1.94-2.06 (br.s., 3 H) 3.35-3.71 (m, 8 H) 3.99 (s, 3 H) 8.06 (br.s., 1 H) 8.23 (br.s., 1 H) 8.27 (d, 1 H) 8.30 (br.s., 1 H) 8.35 (br.s., 1 H) 8.49 (dd, 1 H) 9.08 (br.s., 2 H) 9.45 (s, 1 H) |
| 105 | 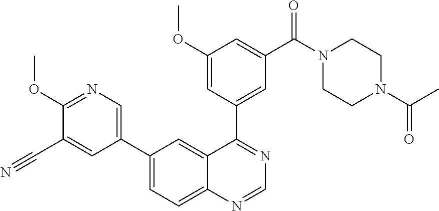 [SMILES: COc1cc(C(=O)N2CCN(C(C)=O)CC2)cc(-c2ncnc3ccc(-c4cnc(OC)c(C#N)c4)cc23)c1] 5-{4-[3-(4-Acetyl-piperazine-1-carbonyl)-5- methoxy-phenyl]-quinazolin-6-yl}-2-methoxy- nicotinonitrile | 1.13 ⁽¹⁾ | 523.3 | ¹H-NMR (400 MHz, DMSO-d₆, 298 K): δ ppm 1.91-2.02 (m, 3 H) 3.39-3.64 (m, 8 H) 3.90 (s, 3 H) 4.06 (s, 3 H) 7.23 (s, 1 H) 7.42 (s, 1 H) 7.50 (s, 1 H) 8.22 (d, 1 H) 8.36 (d, 1 H) 8.43 (d, 1 H) 8.79 (br. s., 1 H) 8.90 (s, 1 H) 9.40 (s, 1 H) |
| 106 | 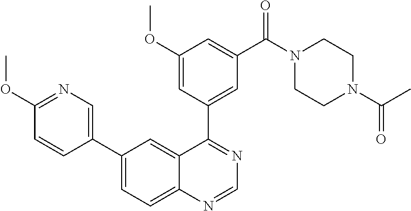 [SMILES: COc1cc(C(=O)N2CCN(C(C)=O)CC2)cc(-c2ncnc3ccc(-c4ccc(OC)nc4)cc23)c1] 1-(4-{3-Methoxy-5-[6-(6-methoxy-pyridin-3-yl)- quinazolin-4-yl]-benzoyl}-piperazin-1-yl)- ethanone | 1.09 ⁽¹⁾ | 498.3 | ¹H-NMR (400 MHz, DMSO-d₆, 298 K): δ ppm 1.89-2.08 (m, 3 H) 3.39-3.69 (m, 8 H) 3.90 (s, 3 H) 3.91 (s, 3 H) 6.96 (d, 1 H) 7.23 (s, 1 H) 7.42 (s, 1 H) 7.49 (s, 1 H) 8.12 (d, 1 H) 8.20 (d, 1 H) 8.25 (s, 1 H) 8.40 (dd, 1 H) 8.59 (d, 1 H) |
| 107 | 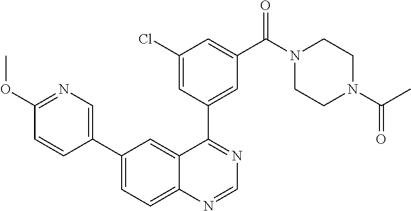 [SMILES: COc1ccc(-c2ccc3ncnc(-c4cc(Cl)cc(C(=O)N5CCN(C(C)=O)CC5)c4)c3c2)cn1] 1-(4-{3-Chloro-5-[6-(6-methoxy-pyridin-3-yl)- quinazolin-4-yl]-benzoyl}-piperazin-1-yl)- ethanone | 1.18 ⁽¹⁾ | 502.3 | ¹H-NMR (400 MHz, DMSO-d₆, 298 K): δ ppm 1.88-2.07 (m, 3 H) 3.39-3.69 (m, 8 H) 3.92 (s, 3 H) 6.97 (d, 1 H) 7.77 (s, 1 H) 7.85 (s, 1 H) 8.02 (s, 1 H) 8.15 (d, 1 H) 8.19- 8.25 (m, 2 H) 8.42 (dd, 1 H) 8.62 (d, 1 H) 9.39 (s, 1 H) |
| 108 | 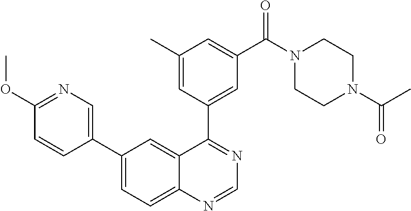 [SMILES: COc1ccc(-c2ccc3ncnc(-c4cc(C)cc(C(=O)N5CCN(C(C)=O)CC5)c4)c3c2)cn1] 1-(4-{3-[6-(6-Methoxy-pyridin-3-yl)-quinazolin- 4-yl]-5-methyl-benzoyl}-piperazin-1-yl)- ethanone | 1.12 ⁽¹⁾ | 482.3 | ¹H-NMR (400 MHz, DMSO-d₆, 298 K): δ ppm 1.87-2.06 (m, 3 H) 3.36-3.71 (m, 8 H) 3.91 (s, 3 H) 6.96 (d, 1 H) 7.50 (s, 1 H) 7.67 (s, 1 H) 7.79 (s, 1 H) 8.11 (dd, 1 H) 8.16-8.24 (m, 2 H) 8.38 (dd, 1 H) 8.59 (d, 1 H) 9.36 (s, 1 H) |
| 109 | 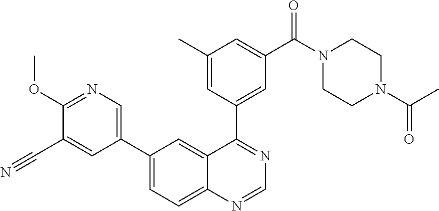 [SMILES: COc1ncc(-c2ccc3ncnc(-c4cc(C)cc(C(=O)N5CCN(C(C)=O)CC5)c4)c3c2)cc1C#N] 5-{4-[3-(4-Acetyl-piperazine-1-carbonyl)-5- methyl-phenyl]-quinazolin-6-yl}-2-methoxy- nicotinonitrile | 1.16 ⁽¹⁾ | 507.3 | ¹H-NMR (400 MHz, DMSO-d₆, 298 K): δ ppm 1.88-2.06 (m, 3 H) 3.35-3.69 (m, 8 H) 4.07 (s, 3 H) 7.51 (s, 1 H) 7.67 (s, 1 H) 7.81 (s, 1 H) 8.23 (d, 1 H) 8.34 (d, 1 H) 8.43 (dd, 1 H) 8.79 (d, 1 H) 8.90 (d, 1 H) 9.39 (s, 1 H) |

⁽¹⁾ LC methode 1,
⁽²⁾ LC methode 2

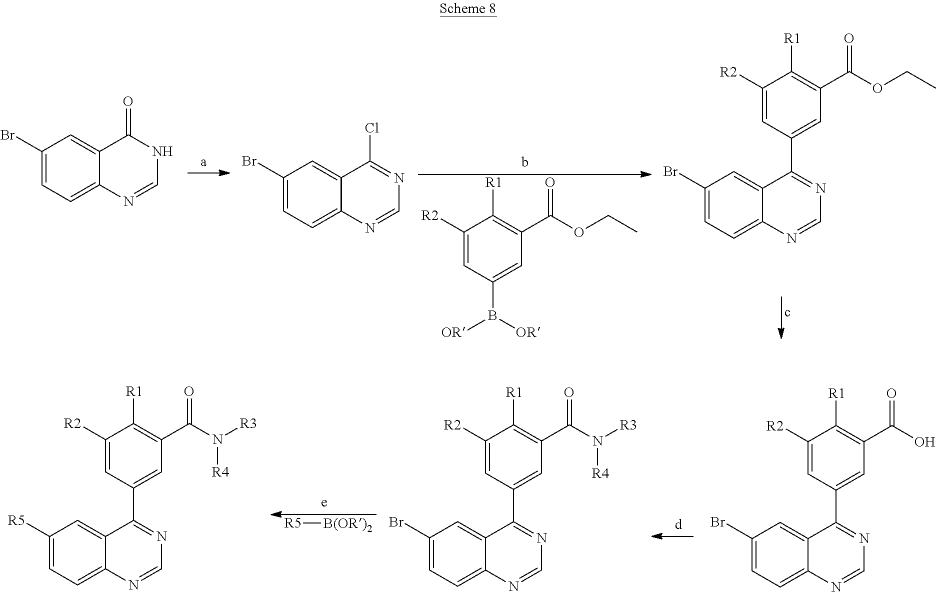

a) Chloronation of 6-Bromo-3H-quinazolin-4-one is performed under customary phophorus oxychoride condition by heating at reflux or 130° C. in diluted (such as in CH2Cl2) or neat phophorus oxychoride. b) Suzuki cross-coupling between 6-Bromo-4-chloro-quinazoline and 3-(ethoxycarbonyl)phenyl-boronic acid or 3-(ethoxycarbonyl)phenyl-boronate is performed under customary Suzuki conditions using palladium catalyst such as preferably Dichlorodiphenylphosphine palladium (PdCl₂(PPh₃)₂), aqueous base and organic solvent such as preferably acetonitrile. The reaction is preferably stirred at a temperature of approximately 100-120° C. in preferably a microwaves oven. The reaction may preferably carried out under an inert gas such as nitrogen or atrgon. c) Saponification of the carboxylic ester was performed under customary saponification conditions, using amoung the possible aqueous bases lithium hydroxyide is preferred and organic solvent such a preferably dioxane. The reation may preferably be carried out at room temperature. d) Condenation of a carboxylic acid with amines of the formula R3NHR4 preferably takes place under customary condensation conditions. The reaction can be carried on by dissolving the carboxylic acid and the amine of formula R3NHR4 in a suitable solvent , for example halogenated hydrocarbon, such as methylene chloride, N,N-dimethylformamide, N, N-dimethylacetamide, N-2-methyl-pyrrolidone, methylene chloride, or a mixture of two or more such solvents, and by the addition of a suitable base, for example triethylamine, diisopropylethylamine (DIPEA) or N-methylmorpholine and a suitable coupling agent that forms a reactive derivative of the carboxylic acid in situ, for example and preferably (2-(1 H-Benzotriazole-1-yl)-1,1,3,3-tetramethyluronium hexafluorophosphate (HBTU). The reaction mixture is preferably stirred at a temperature of from approximately −20 to 50° C., especially from −5° C. to 30° C., e.g at 0° C. to room temperature. The reaction my preferably be carried out under an inert gas, e.g. nitrogenor argon. e) Suzuki cross-coupling between aryl bromide and boronic acid or boronic acid derivatives such as boronate of formula R5—B(OR′)₂ is performed under customary Suzuki conditions using palladium catalyst such as preferably palladium tetrakis(triphenylphosphine) palladium (Pd(PPh₃)₄), aqueous base and organic solvent such as preferably acetonitrile. The reaction is preferably stirred at a temperature of approximately 100-120° C. in preferably a microwaves oven. The reaction may preferably carried out under an inert gas such as nitrogen or argon.

The final compounds described herein were according the general procedure described in scheme 8.

### Example 110

### 1-(4-{3-Fluoro-5-[6-(6-methoxy-pyridin-3-yl)-quinazolin-4-yl]-benzoyl}-piperazin-1-yl)-ethanone

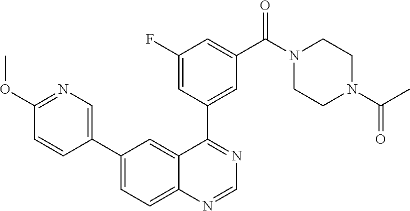 [SMILES: COc1ccc(-c2ccc3ncnc(-c4cc(F)cc(C(=O)N5CCN(C(C)=O)CC5)c4)c3c2)cn1]

A mixture of 1-{4-[3-(6-bromo-quinazolin-4-yl)-5-fluoro-benzoyl]-piperazin-1-yl}-ethanone (150 mg, 0.328 mmol), 6-methoxypyridin-3-ylboronic acid (50.2 mg, 0.328 mmol), K₃PO₄ (104 mg, 0.492 mmol) and PdCl₂(PPh₃)₂ (11.51 mg, 0.016 mmol) was flushed with argon for few minutes. To the mixture was then added 3 ml of Acetonitrile followed by 0.3 ml of water. The vial was capped and the reaction mixture was heated to 120° C. for 10 min using a microwave oven. The mixture was then cooled down to rt, diluted with CH₂Cl₂ and filtered through a Celite pad. The organic layer was washed with sat. Bicarbonate solution, dried by passing through a phase separating cartridge and evaporated. Purification by preparative reverse phase Gilson HPLC and subsequent neutralization of the combined fractions over SCx-2 cartridge gave the title compound (68 mg, 41% yield) as a powder. ¹H-NMR (400 MHz, DMSO-d₆, 298 K): δ ppm 1.88-2.06 (m, 3H) 3.34-3.69 (m, 8H) 3.91 (s, 3H) 6.97 (d, 1 H) 7.58 (d, 1H) 7.73 (s, 1H) 7.85 (dd, 1H) 8.15 (d, 1H) 8.19-8.26 (m, 2H) 8.42 (dd, 1H) 8.62 (d, 1H) 9.39 (s, 1H). MS: 485.0 [M+1]⁺, Rt⁽¹⁾=1.04 min.

### 1-{4-[3-(6-Bromo-quinazolin-4-yl)-5-fluoro-benzoyl]-piperazin-1-yl}-ethanone

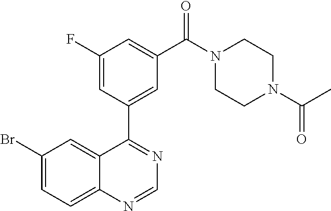 [SMILES: CC(=O)N1CCN(C(=O)c2cc(F)cc(-c3ncnc4ccc(Br)cc34)c2)CC1]

To a mixture of 3-(6-bromo-quinazolin-4-yl)-5-fluoro-benzoic acid (1.36 g, 3.72 mmol) in CH₂Cl₂ (15 mL) was added DIPEA (1.30 mL, 7.44 mmol) and HBTU (1.694 g, 4.47 mmol) at rt. The reaction mixture was stirred at rt for 20 min. To the mixture was then added 1-(piperazin-1-yl)ethanone (0.572 g, 4.47 mmol) and the reaction mixture was stirred at rt for 1 h. The reaction was quenched with a saturated aqueous solution of NaHCO₃ and extracted with CH₂Cl₂. The organic layer was washed twice with brine, dried by passing through a phase separating cartridge and evaporated. Purification by Flash chromatography using Biotage Isolera system (amine functionalized silica KP-NH, eluting with Cyclohexane/EtOAc 0 to 100%) gave the title compound (1.20 g, 68% yield) as a beige foam. MS: 457.4-459.3 [M+1]⁺, Rt⁽²⁾=1.03 min.

### 3-(6-Bromo-quinazolin-4-yl)-5-fluoro-benzoic acid

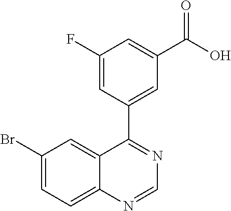 [SMILES: O=C(O)c1cc(F)cc(-c2ncnc3ccc(Br)cc23)c1]

To a solution of 3-(6-bromo-quinazolin-4-yl)-5-fluoro-benzoic acid ethyl ester (1.417 g, 3.78 mmol) in dioxane (15 mL) was added at rt a 2M aqueous solution of LiOH.H₂O (7.55 mL, 7.55 mmol) and the reaction mixture was stirred 2 h at rt. The reaction was quenched with a 2M aqueous solution of HCl (5 mL), the formed precipitate was filtered and dried under vacuum to give the title compound (1.36 g, 99% yield) as a white solid. MS: 349.0 [M+1]⁺, Rt⁽¹⁾=1.17 min.

### 3-(6-Bromo-quinazolin-4-yl)-5-fluoro-benzoic acid ethyl ester

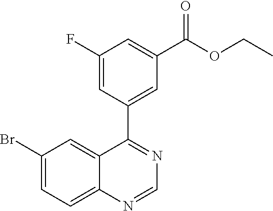 [SMILES: CCOC(=O)c1cc(F)cc(-c2ncnc3ccc(Br)cc23)c1]

A mixture of 6-bromo-4-chloroquinazoline (1.5 g, 6.16 mmol), 3-(ethoxycarbonyl)-5-fluorophenylboronic acid (1.306 g, 6.16 mmol), K₃PO₄ (1.961 g, 9.24 mmol) and PdCl₂(PPh₃)₂ (216 mg, 0.308 mmol) was flushed with argon for few minutes. To the mixture was then added 24 ml of Acetonitrile followed by 2.4 ml of water. The vial was capped and the reaction mixture was heated to 120° C. for 10 min using a microwave oven. The mixture was then cooled down to rt, diluted with CH₂Cl₂ and filtered through a Celite pad. The organic layer was washed with sat. Bicarbonate solution, dried by passing through a phase separating cartridge and evaporated. Purification by Flash chromatography using Biotage Isolera system (amine functionalized silica KP-NH, eluting with Cyclohexane/EtOAc 0 to 30%) gave the title compound (1.417 g, 61% yield) as a solid. MS: 375.1-377.1 [M+1]⁺, Rt⁽¹⁾=1.54 min.

The compound of example 111 was prepared using procedures analogous to those used for example 110, using appropriate starting materials.

| | | | MS (ES): | |
|---|---|---|---|---|
| Example | Structure/Name | Rt (min.) | [M + H]⁺ | 1H-NMR |
| 111 | 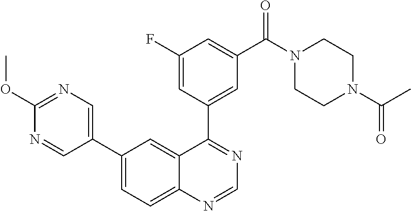 [SMILES: COc1ncc(-c2ccc3ncnc(-c4cc(F)cc(C(=O)N5CCN(C(C)=O)CC5)c4)c3c2)cn1] 1-(4-{3-Fluoro-5-[6-(2-methoxy-pyrimidin-5-yl)- quinazolin-4-yl]-benzoyl}-piperazin-1-yl)- ethanone | 0.91 ⁽¹⁾ | 487.3 | ¹H-NMR (400 MHz, DMSO-d₆, 298 K): δ ppm 2.00 (d, 3 H) 3.35-3.74 (m, 8 H) 3.99 (s, 3 H) 7.58 (d, 1 H) 7.74 (s, 1 H) 7.85 (d, 1 H) 8.24 (d, 1 H) 8.35 (s, 1 H) 8.46 (d, 1 H) 9.07 (s, 2 H) 9.41 (s, 1 H) |

⁽¹⁾ LC methode 1

### Example 112

### 1-(4-{4-[6-(2-Methoxy-pyrimidin-5-yl)-quinazolin-4-yl]-pyridine-2-carbonyl}-piperazin-1-yl)-ethanone

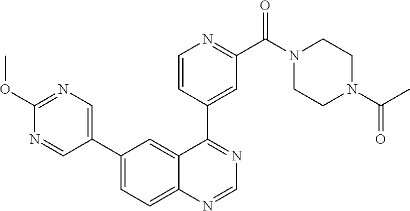 [SMILES: COc1ncc(-c2ccc3ncnc(-c4ccnc(C(=O)N5CCN(C(C)=O)CC5)c4)c3c2)cn1]

To a mixture of 2-methoxypyrimidin-5-yl boronic acid (36.9 mg, 0.240 mmol) and Pd(PPh₃)₄ (11.56 mg, 0.010 mmol) was added a solution of 1-{4-[4-(6-Bromo-quinazolin-4-yl)-pyridine-2-carbonyl]-piperazin-1-yl}-ethanone (88 mg, 0.200 mmol) in 2 mL of acetonitrile. The reaction mixture was flushed with argon and a 1M aqueous solution of Na₂CO₃ (0.400 mL, 0.400 mmol) was added and the vial capped. The reaction mixture was heated to 120° C. for 10 min using a microwave oven then cooled down to rt, diluted with EtOAc, filtered through a Celite pad and concentrated. Purification by preparative reverse phase Gilson HPLC and subsequent neutralization of the combined fractions over PL-HCO₃ MP gave the title compound (40 mg, 43% yield) as a white powder. ¹H-NMR (400 MHz, DMSO-d₆, 298 K): δ ppm 2.01-2.06 (d, 3H) 3.48 (br.s., 3H) 3.58 (br.s., 3H) 3.65 (br.s., 1H) 3.73 (br.s., 1H) 3.99 (s, 3H) 8.01 (dd, 1H) 8.06 (br.s., 1H) 8.28 (d, 1H) 8.34 (d, 1H) 8.49 (dd, 1H) 8.87 (d, 1H) 9.09 (s, 2H) 9.47 (s, 1H). MS: 470.6 [M+1]⁺, Rt⁽²⁾=0.78 min.

The compounds of examples 113 and 114 were prepared using procedures analogous to those used for example 112, using appropriate starting materials.

| | | | MS (ES): | |
|---|---|---|---|---|
| Example | Structure/Name | Rt (min.) | [M + H]⁺ | 1H-NMR |
| 113 | 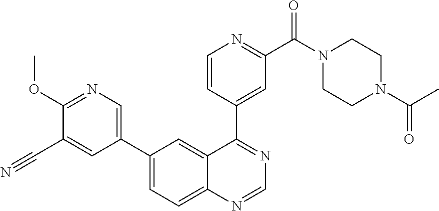 [SMILES: COc1ncc(-c2ccc3ncnc(-c4ccnc(C(=O)N5CCN(C(C)=O)CC5)c4)c3c2)cc1C#N] 5-{4-[2-(4-Acetyl-piperazine-1-carbonyl)- pyridin-4-yl]-quinazolin-6-yl}-2-methoxy- nicotinonitrile | 0.97 ⁽²⁾ | 494.6 | ¹H-NMR (400 MHz, DMSO-d₆, 298 K): δ ppm 2.00-2.05 (br.s., 3 H) 3.44-3.75 (m, 8 H) 4.07 (s, 3 H) 8.01 (d, 1 H) 8.06 (br.s., 1 H) 8.27 (d, 1 H) 8.35 (d, 1 H) 8.46-8.51 (m, 1 H) 8.82 (br.s., 1 H) 8.88 (d, 1 H) 8.95 (br.s., 1 H) 9.47 (s, 1 H) |
| 114 | 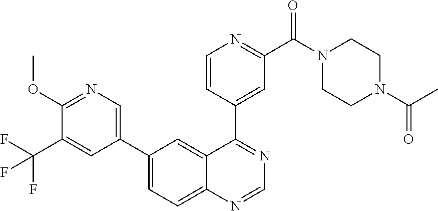 [SMILES: COc1ncc(-c2ccc3ncnc(-c4ccnc(C(=O)N5CCN(C(C)=O)CC5)c4)c3c2)cc1C(F)(F)F] 1-(4-{4-[6-(6-Methoxy-5-trifluoromethyl-pyridin- 3-yl)-quinazolin-4-yl]-pyridine-2-carbonyl}- piperazin-1-yl)-ethanone | 1.13 ⁽²⁾ | 537.6 | ¹H-NMR (400 MHz, DMSO-d₆, 298 K): δ ppm 1.96-2.08 (d, 3 H) 3.44-3.75 (m, 8 H) 4.06 (s, 3 H) 8.02 (dd, 1 H) 8.08 (br.s., 1 H) 8.26 (d, 1 H) 8.34 (d, 1 H) 8.50-8.53 (dd, 2 H) 8.87 (d, 1 H) 8.90 (d, 1 H) 9.47 (s, 1 H) |

⁽²⁾ LC methode 2

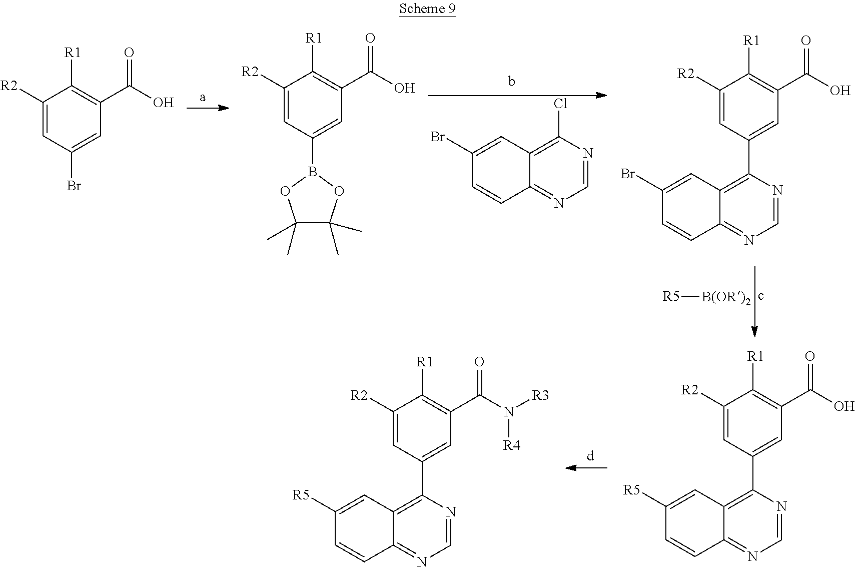

a) Formation of the boronate ester was performed using palladium catalyst such as preferably 1,1-Bis(diphenylphosphino)-ferrocene[-dichloropalladium (PdCl2(dppf)-CH₂Cl₂), aqueous base such as preferably potassium acetate organic solvent such as preferably dioxane and Bis-(pinacolato)-diboron. The reaction is preferably stirred at approximately 80° C. for several hours. b) Suzuki cross-coupling between 6-Bromo-4-chloro-quinazoline and the boronate is performed under customary Suzuki conditions using Dichlorodiphenylphosphine palladium (PdCl₂(PPh₃)₂), aqueous base and organic solvent such as preferably acetonitrile. The reaction is preferably stirred at a temperature of approximately 100-120° C. in preferably a microwaves oven. The reaction may preferably carried out under an inert gas such as nitrogen or atrgon. d) Suzuki cross-coupling between aryl bromide and boronic acid or boronic acid derivatives such as boronate of formula R5-B(OR′)₂ is performed under customary Suzuki conditions using palladium catalyst such as preferably Dichlorodiphenylphosphine palladium (PdCl₂(PPh₃)₂), aqueous base and organic solvent such as preferably acetonitrile. The reaction is preferably stirred at a temperature of approximately 100-120° C. in preferably a microwaves oven. The reaction may preferably carried out under an inert gas such as nitrogen or argon. c) Suzuki cross-coupling between aryl bromide and boronic acid or boronic acid derivatives such as boronate of formula R5-B(OR′)₂ is performed under customary Suzuki conditions using palladium catalyst such as preferably palladium tetrakis(triphenylphosphine) palladium (Pd(PPh₃)₄), aqueous base and organic solvent such as preferably acetonitrile. The reaction is preferably stirred at a temperature of approximately 100-120° C. The reaction may preferably carried out under an inert gas such as nitrogen or argon. d) Condenation of a carboxylic acid with amines of the formula R3NHR4 preferably takes place under customary condensation conditions. The reaction can be carried on by dissolving the carboxylic acid and the amine of formula R3NHR4 in a suitable solvent, for example halogenated hydrocarbon, such as methylene chloride, N, N-dimethylformamide, N,N-dimethylacetamide, N-2-methyl-pyrrolidone, methylene chloride, or a mixture of two or more such solvents, and by the addition of a suitable base, for example triethylamine, diisopropylethylamine (DIPEA) or N-methylmorpholine and a suitable coupling agent that forms a reactive derivative of the carboxylic acid in situ, for example (2-(1H-Benzotriazole-1-yl)-1,1,3,3-tetramethyluronium hexafluorophosphate (HBTU) and preferably Propylphosphonic anhydride. The reaction mixture is preferably stirred at a temperature of from approximately −20 to 50° C., especially from −5° C. to 30° C., e.g at 0° C. to room temperature. The reaction my preferably be carried out under an inert gas, e.g. nitrogenor argon.

The final compounds described herein were according the general procedure described in scheme 9.

### Example 115

### {5-[6-(6-Methoxy-pyridin-3-yl)-quinazolin-4-yl]-2-methyl-phenyl}-(4-methyl-piperazin-1-yl)-methanone

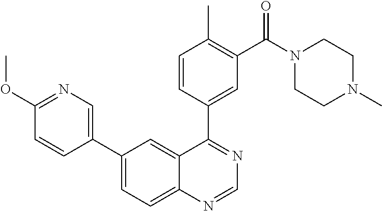 [SMILES: COc1ccc(-c2ccc3ncnc(-c4ccc(C)c(C(=O)N5CCN(C)CC5)c4)c3c2)cn1]

To a mixture of 5-[6-(6-methoxy-pyridin-3-yl)-quinazolin-4-yl]-2-methyl-benzoic acid (55 mg, 0.148 mmol) in CH₂Cl₂ (2 mL) was added DIPEA (0.039 mL, 0.222 mmol) and propylphosphonic anhydride sol. 50% in DMF (0.065 mL, 0.222 mmol) at rt. The reaction mixture was stirred at rt for 30 min. To the mixture was then added 1-methylpiperazine (0.016 mL, 0.148 mmol) and the reaction mixture was stirred at rt for 12 h. The reaction was quenched with a saturated aqueous solution of NaHCO₃ and extracted with CH₂Cl₂. The organic layer was then dried by passing through a phase separating cartridge and evaporated. Purification by preparative reverse phase HPLC and subsequent neutralization of the combined fractions over SCx-2 cartridge gave the title compound (32 mg, 46% yield) as a yellow powder. ¹H-NMR (400 MHz, DMSO-d₆, 298 K): δ ppm 2.13 (s, 3H) 2.17 (br. s., 2 H) 2.34 (d, 2H) 2.36 (s, 3H) 3.26 (t, 2H) 3.65 (br. s., 2H) 3.92 (s, 3H) 6.96 (d, 1H) 7.56 (d, 1H) 7.64 (d, 1H) 7.87 (dd, 1H) 8.12 (dd, 1H) 8.19 (d, 1H) 8.23 (d, 1H) 8.38 (dd, 1H) 8.59 (d, 1H) 9.36 (s, 1H). MS: 454.3 [M+1]⁺, Rt⁽¹)=: 0.87 min.

### 5-[6-(6-Methoxy-pyridin-3-yl)-quinazolin-4-yl]-2-methyl-benzoic acid

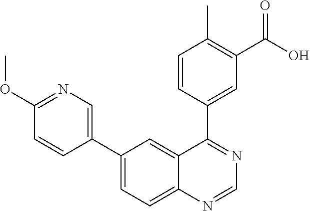 [SMILES: COc1ccc(-c2ccc3ncnc(-c4ccc(C)c(C(=O)O)c4)c3c2)cn1]

A mixture of 5-(6-bromo-quinazolin-4-yl)-2-methyl-benzoic acid (318 mg, 0.741 mmol), 6-methoxypyridin-3-ylboronic acid (113 mg, 0.741 mmol), K₃PO₄ (236 mg, 1.112 mmol) and PdCl₂(PPh₃)₂ (26.0 mg, 0.037 mmol) was flushed with argon for few minutes. To the mixture was then added 6 ml of Acetonitrile followed by 0.8 ml of water. The vial was capped and the reaction mixture was heated to 120° C. for 5 min using a microwave oven. The mixture was then cooled down to rt, diluted with EtOAc and filtered through a Celite pad. The organic layer was washed with a solution of HCl 2M. As a part of the compound remains into the aqueous phase, the pH was basified around 8 and the compound was extracted again with EtOAc. The combined organic layers were dried over Na₂SO₄, filtered and evaporated. Precipitation in EtOAc/Cyclohexane gave the title compound (168 mg, 61% yield) as a yellow powder. MS: 372.2 [M+1]⁺. Rt⁽¹⁾=1.22 min.

### 5-(6-Bromo-quinazolin-4-yl)-2-methyl-benzoic acid

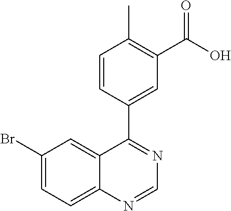 [SMILES: Cc1ccc(-c2ncnc3ccc(Br)cc23)cc1C(=O)O]

A mixture of 6-bromo-4-chloroquinazoline (300 mg, 1.232 mmol), 2-methyl-5-(4,4,5,5-tetramethyl-[1,3,2]dioxaborolan-2-yl)-benzoic acid (497 mg, 1.232 mmol, purity 65% by UPLC), K₃PO₄ (392 mg, 1.848 mmol) and PdCl₂(PPh₃)₂ (43.2 mg, 0.062 mmol) was flushed with argon for few minutes. To the mixture was then added 8 ml of Acetonitrile followed by 0.8 ml of water. The vial was capped and the reaction mixture was heated to 120° C. for 5 min using a microwave oven. The mixture was then cooled down to rt, diluted with EtOAc and filtered through a Celite pad. The organic layer was washed with a solution of HCl 2M, dried over Na₂SO₄, filtered and evaporated. Precipitation in EtOAc/Cyclohexane gave the title compound (318 mg, 80% purity, 60% yield) as a beige powder. MS: 345.0 [M+1]⁺, Rt⁽¹⁾=1.23 min.

Examples 116 to 117, were prepared using procedures analogous to those used for example 115, using appropriate starting materials.

| | | | MS (ES): | |
|---|---|---|---|---|
| Example | Structure/Name | Rt (min.) | [M + H]⁺ | 1H-NMR |
| 116 | 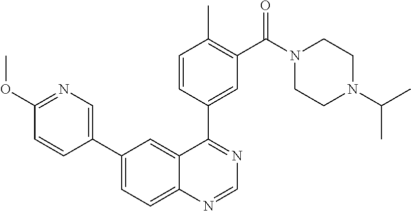 [SMILES: COc1ccc(-c2ccc3ncnc(-c4ccc(C)c(C(=O)N5CCN(C(C)C)CC5)c4)c3c2)cn1] (4-Isopropyl-piperazin-1-yl)-{5-[6-(6-methoxy- pyridin-3-yl)-quinazolin-4-yl]-2-methyl-phenyl}- methanone | 0.92 ⁽¹⁾ | 482.4 | ¹H-NMR (400 MHz, DMSO-d₆, 298 K): δ ppm 0.89 (d, 6 H) 2.28 (t, 2 H) 2.35 (s, 3 H) 2.42-2.48 (m, 2 H) 3.25 (d, 2 H) 3.47-3.76 (m, 2 H) 3.91 (s, 3 H) 6.95 (d, 1 H) 7.56 (d, 1 H) 7.62 (d, 1 H) 7.85 (d, 1 H) 8.08-8.15 (m, 1 H) 8.17-8.24 (m, 2 H) 8.34-8.41 (m, 1 H) 8.59 (d, 1 H) 9.36 (s, 1 H) |
| 117 | 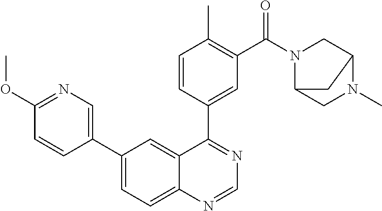 [SMILES: COc1ccc(-c2ccc3ncnc(-c4ccc(C)c(C(=O)N5CC6CC5CN6C)c4)c3c2)cn1] (2,5-Diaza-bicyclo[2.2.1]hept-2-yl)-{5-[6-(6- methoxy-pyridin-3-yl)-quinazolin-4-yl]-2-methyl- phenyl}-methanone | 0.88 ⁽¹⁾ | 466.3 | ¹H-NMR (400 MHz, DMSO-d₆, 298 K): δ ppm 1.65-1.88 (m, 2 H) 2.21-2.27 (m, 3 H) 2.36-2.40 (m, 3 H) 2.53- 3.23 (m, 4 H) 3.40- 3.55 (m, 2 H) 3.90-3.93 (m, 3 H) 6.93-7.00 (m, 1 H) 7.52-7.61 (m, 1 H) 7.67- 7.73 (m, 1 H) 7.82- 7.90 (m, 1 H) 8.08-8.15 (m, 1 H) 8.17-8.24 (m, 1 H) 8.33-8.41 (m, 1 H) 8.56- 8.61 (m, 1 H) 9.34- 9.38 (m, 1 H) |

⁽¹⁾ LC/MS methode 1

### Biological Evaluation

### Biological Assays

### 1 Determination of Enzymatic PI3K Alpha and PI3K Delta Isoform Inhibition

### 1.1 Test of Lipid Kinase Activity

The efficacy of the compounds of examples 1-117 as PI3 kinase inhibitors can be demonstrated as follows:

The kinase reaction is performed in a final volume of 50 μl per well of a half area COSTAR, 96 well plate. The final concentrations of ATP and phosphatidyl inositol in the assay are 5 μM and 6 μg/ml, respectively. The reaction is started by the addition of PI3 kinase, e.g. PI3 kinase δ.

p110δ. The components of the assay are added per well as follows:
10 μl test compound in 5% DMSO per well in columns 2-1.
Total activity is determined by addition 10 μl of 5% vol/vol DMSO in the first 4 wells of column 1 and the last 4 wells of column 12.
The background is determined by addition of 10 μM control compound to the last 4 wells of column 1 and the first 4 wells of column 12.
2 mL ‘Assay mix’ are prepared per plate:
   1.912 ml of HEPES assay buffer
   8.33 μl of 3 mM stock of ATP giving a final concentration of 5 μM per well
   1 μl of [³³P]ATP on the activity date giving 0.05 μCi per well
   30 μl of 1 mg/ml PI stock giving a final concentration of 6 μg/ml per well
   5 μl of 1 M stock MgCl₂ giving a final concentration of 1 mM per well
20 μl of the assay mix are added per well.
2 ml ‘Enzyme mix’ are prepared per plate (x* μl PI3 kinase p110β in 2 ml of kinase buffer). The ‘Enzyme mix’ is kept on ice during addition to the assay plates.
20 μl ‘Enzyme mix’ are added/well to start the reaction.
The plate is then incubated at room temperature for 90 minutes.
The reaction is terminated by the addition of 50 μl WGA-SPA bead (wheat germ agglutinin-coated Scintillation Proximity Assay beads) suspension per well.
The assay plate is sealed using TopSeal-S (heat seal for polystyrene microplates, PerkinElmer LAS [Deutschland] GmbH, Rodgau, Germany) and incubated at room temperature for at least 60 minutes.
The assay plate is then centrifuged at 1500 rpm for 2 minutes using the Jouan bench top centrifuge (Jouan Inc., Nantes, France).
The assay plate is counted using a Packard TopCount, each well being counted for 20 seconds.
   * The volume of enzyme is dependent on the enzymatic activity of the batch in use.

In a more preferred assay, the kinase reaction is performed in a final volume of 10 μl per well of a low volume non-binding CORNING, 384 well black plate (Cat. No. #3676). The final concentrations of ATP and phosphatidyl inositol (PI) in the assay are 1 μM and 10 μg/ml, respectively. The reaction is started by the addition of ATP.

The components of the assay are added per well as follows:

50 nl test compounds in 90% DMSO per well, in columns 1-20, 8 concentrations (1/3 and 1/3.33 serial dilution step) in single.
Low control: 50 nl of 90% DMSO in half the wells of columns 23-24 (0.45% in final).
High control: 50 nl of reference compound (e.g. compound of Example 7 in WO 2006/122806) in the other half of columns 23-24 (2.5 μM in final).
Standard: 50 nl of reference compound as just mentioned diluted as the test compounds in columns 21-22.
20 ml ‘buffer’ are prepared per assay:
   200 μl of 1M TRIS HCl pH7.5 (10 mM in final)
   60 μl of 1M MgCl₂ (3 mM in final)
   500 μl of 2M NaCl (50 mM in final)
   100 μl of 10% CHAPS (0.05% in final)
   200 μl of 100 mM DTT (1 mM in final)
   18.94 ml of nanopure water
10 ml ‘PI’ are prepared per assay:
   200 μl of 1 mg/ml 1-alpha-Phosphatidylinositol (Liver Bovine, Avanti Polar Lipids Cat. No. 840042C MW=909.12) prepared in 3% OctylGlucoside (10 μg/ml in final)
   9.8 ml of ‘buffer’
10 ml ‘ATP’ are prepared per assay:
   6.7 μl of 3 mM stock of ATP giving a final concentration of 1 μM per well
   10 ml of ‘buffer’
2.5 ml of each PI3K construct are prepared per assay in ‘PI’ with the following final concentration:
   10 nM PI3K alfa EMV B1075
   25 nM beta EMV BV949
   10 nM delta EMV BV1060
   150 nM gamma EMV BV950
5 μl of ‘PI/PI3K’ are added per well.
5 μl ‘ATP’ are added per well to start the reaction.
The plates are then incubated at room temperature for 60 minutes (alfa, beta, delta) or 120 minutes (gamma).
The reaction is terminated by the addition of 10 μl Kinase-Glo (Promega Cat. No. #6714).
The assay plates are read after 10 minutes in Synergy 2 reader (BioTek, Vermont USA) with an integration time of 100 milliseconds and sensitivity set to 191.
Output: The High control is around 60′000 counts and the Low control is 30,000 or lower
This luminescence assay gives a useful Z′ ratio between 0.4 and 0.7

The Z′ value is a universal measurement of the robustness of an assay. A Z′ between 0.5 and 1.0 is considered an excellent assay.

For this assay, the PI3K constructs mentioned are prepared as follows:

### 1.2 Generation of Gene Constructs

Two different constructs, BV 1052 and BV 1075, are used to generate the PI3 Kinase α proteins for compound screening.

PI3Kα BV-1052 p85(iSH2)-Gly linker-p110a (D20aa)-C-term His tag

PCR products for the inter SH2 domain (iSH2) of the p85 subunit and for the p110-a subunit (with a deletion of the first 20 amino acids) are generated and fused by overlapping PCR. The iSH2 PCR product is generated from first strand cDNA using initially primers gwG130-p01 (5′-CGAGAATATGATAGATTATATGAAGAAT-3′) (SEQ ID NO: 1) and gwG130-p02 (5′-TGGTTT-AATGCTGTTCATACGTTTGTCAAT-3′) (SEQ ID NO: 2). Subsequently in a secondary PCR reaction, Gateway (Invitrogen AG, Basel, Switzerland) recombination AttB1 sites and linker sequences are added at the 5′ end and 3′ end of the p85 iSH2 fragment respectively, using primers gwG130-p03 (5′-GGGACAAGTTTGTACAAAAAAGCAGGCTACGAAGGAGATATACATAT-GCGAGAATATGATAGATTATATGAAGAAT-3′) (SEQ ID NO: 3) and gwG152-p04 (5′-TACCATAATTCCACCACCACCACCGGAAATTCCCCCTGGTTT-AATGCTGTTCATACGTTTGTCAAT-3′) (SEQ ID NO: 4).

The p110-a fragment is also generated from first strand cDNA, initially using primers gwG152-p01 (5′-CTAGTGGAATGTTTACTACCAAATGG-3′) (SEQ ID NO: 5) and gwG152-p02 (5′-GTTCAATG-CATGCTGTTTAATTGTGT-3′) (SEQ ID NO: 6).

In a subsequent PCR reaction, linker sequence and a Histidine tag are added at the 5′ end and 3′ end of the p110-a fragment respectively, using primers gw152-p03 (5′-GGGGGAATTTCCGGTGGTGGTGGTGGAATTATGGTAC-TAGTGGAATGTTTACTACC-AAATGGA-3′) (SEQ ID NO: 7) and gwG152-p06 (5′-AGCTCCGTGATGGTGATGGTGATGTGCTCCGTTCAATG-CATGCTGTTTAATTGTGT-3′) (SEQ ID NO: 8).

The p85-iSH2/p110-a fusion protein is assembled in a third PCR reaction by the overlapping linkers at the 3′ end of the iSH2 fragment and the 5′ end of the p110-a fragment, using the above mentioned gwG130-p03 primer and a primer containing an overlapping Histidine tag and the AttB2 recombination sequences (5′-GGGACCACTTTGTACAAGAAAGCTGGGTTTAAGCTCCGTGATGGTGATGGTGAT-GTGCTCC-3′) (SEQ ID NO: 9).

This final product is recombined in a (Invitrogen) OR reaction into the donor vector pDONR201 to generate the ORF318 entry clone. This clone is verified by sequencing and used in a Gateway LR reaction to transfer the insert into the Gateway adapted pBlueBac4.5 (Invitrogen) vector for generation of the baculovirus expression vector LR410.

PI3Kα BV-1075 p85(iSH2)-12 XGly linker-p110a (D20aa)-C-term His tag The construct for Baculovirus BV-1075 is generated by a three-part ligation comprised of a p85 fragment and a p110-a fragment cloned into vector pBlueBac4.5. The p85 fragment is derived from plasmid p1661-2 digested with Nhe/Spe. The p110-a fragment derived from LR410 (see above) as a SpeI/HindIII fragment. The cloning vector pBlueBac4.5 (Invitrogen) is digested with Nhe/HindIII. This results in the construct PED 153.8

The p85 component (iSH2) is generated by PCR using ORF 318 (described above) as a template and one forward primer KAC1028 (5′-GCTAGCATGCGAGAATATGATAGATTATATGAAGAATATACC) (SEQ ID NO: 10) and two reverse primers, KAC1029 (5′-GCCTCCACCACCTCCGCCTGGTTTAATGCTGTTCATACGTTTGTC) (SEQ ID NO: 11) and KAC1039 (5′-TACTAGTCCGCCTCCACCACCTCCGCCTCCACCACCTCCGCC) (SEQ ID NO: 12).

The two reverse primers overlap and incorporate the 12x Gly linker and the N-terminal sequence of the p110a gene to the SpeI site. The 12x Gly linker replaces the linker in the BV1052 construct. The PCR fragment is cloned into pCR2.1 TOPO (Invitrogen). Of the resulting clones, p1661-2 is determined to be correct. This plasmid is digested with Nhe and SpeI and the resulting fragment is gel-isolated and purified for sub-cloning. The p110-a cloning fragment is generated by enzymatic digest of clone LR410 (see above) with Spe I and HindIII. The SpeI site is in the coding region of the p110a gene. The resulting fragment is gel-isolated and purified for sub-cloning.

The cloning vector, pBlueBac4.5 (Invitrogen) is prepared by enzymatic digestion with Nhe and HindIII. The cut vector is purified with Qiagen (Quiagen N.V, Venlo, Netherlands) column and then dephosphorylated with Calf Intestine alkaline phosphatase (CIP) (New England BioLabs, Ipswich, Mass.). After completion of the CIP reaction the cut vector is again column purified to generate the final vector. A 3 part ligation is performed using Roche Rapid ligase and the vendor specifications.

PI3Kδ BV-949 p85(iSH2)-Gly linker-p110b (full-length)-C-term H is tag

PCR products for the inter SH2 domain (iSH2) of the p85 subunit and for the full-length p110-b subunit are generated and fused by overlapping PCR.

The iSH2 PCR product is generated from first strand cDNA initially using primers gwG130-p01 (5′-CGAGAATATGATAGATTATATGAAGAAT-3′) (SEQ ID NO: 1) and gwG130-p02 (5′-TGGTTT-AATGCTGTTCATACGTTTGTCAAT-3′) (SEQ ID NO: 2). Subsequently, in a secondary PCR reaction Gateway (Invitrogen) recombination AttB1 sites and linker sequences are added at the 5′ end and 3′ end of the p85 iSH2 fragment respectively, using primers gwG130-p03 (5′-GGGACAAGTTTGTACAAAAAAGCAGGCTACGAAGGAGATA-TACATATGCGAGAATATGATAGATTATATGAAGAAT-3′) (SEQ ID NO: 3) and gwG130-p05 (5′-ACTGAAGCATCCTCCTCCTCCTCCTCCTGGTTTAAT-GCTGTTCATACGTTTGTC-3′) (SEQ ID NO: 13).

The p110-b fragment is also generated from first strand cDNA initially using primers gwG130-p04 (5′-ATTAAACCAGGAGGAGGAGGAGGAGGATGCTTCAGTTTCATAATGCC-TCCTGCT-3′) (SEQ ID NO: 4) which contains linker sequences and the 5′ end of p110-b and gwG130-p06 (5′-AGCTCCGTGATGGTGATGGTGATGTGCTCCAGATCTGTAGTCTTT-CCGAACTGTGTG-3′) (SEQ ID NO: 14) which contains sequences of the 3′ end of p110-b fused to a Histidine tag.

The p85-iSH2/p110-b fusion protein is assembled by an overlapping PCR a reaction of the linkers at the 3′ end of the iSH2 fragment and the 5′ end of the p110-b fragment, using the above mentioned gwG130-p03 primer and a primer containing an overlapping Histidine tag and the AttB2 recombination sequences (5′-GGGACCACTTTGTACAAGAAAGCTGGGTTT-AAGCTCCGTGATGGTGATGGTGATGTGCTCC-3′) (SEQ ID NO: 15).

This final product is recombined in a Gateway (Invitrogen) OR reaction into the donor vector pDONR201 to generate the ORF253 entry clone. This clone is verified by sequencing and used in a Gateway LR reaction to transfer the insert into the Gateway adapted pBlueBac4.5 (Invitrogen) vector for generation of the baculovirus expression vector LR280.

PI3Kδ BV-1060 p85(iSH2)-Gly linker-p110d (full-length)-C-term His tag

PCR products for the inter SH2 domain (iSH2) of the p85 subunit and for the full-length p110-d subunit are generated and fused by overlapping PCR.

The iSH2 PCR product is generated from first strand cDNA using initially primers gwG130-p01 (5′-CGAGAATATGATAGATTATATGAAGAAT-3′) (SEQ ID NO: 1) and gwG130-p02 (5′-TGGTTT-AATGCTGTTCATACGTTTGTCAAT-3′) (SEQ ID NO: 2). Subsequently, in a secondary PCR reaction Gateway (Invitrogen) recombination AttB1 sites and linker sequences are added at the 5′ end and 3′ end of the p85 iSH2 fragment respectively, using primers gwG130-p03 (5′-GGGACAAGTTTGTACAAAAAAGCAGGCTACGAAGGAGATATACAT-ATGCGAGAATATGATAGATTATATGAAGAAT-3′) (SEQ ID NO: 3) and gwG154-p04 (5′-TCCTCCTCCTCCTCCTCCTGGTTTAATGCTGTTCATACGTTTGTC-3′) (SEQ ID NO: 16).

The p110-a fragment is also generated from first strand cDNA using initially primers gwG154-p01 (5′-ATGCCCCCTGGGGTGGACTGCCCCAT-3′) (SEQ ID NO: 17) and gwG154-p02 (5′-CTACTG-CCTGTTGTCTTTGGACACGT-3′) (SEQ ID NO: 18).

In a subsequent PCR reaction linker sequences and a Histidine tag is added at the 5′ end and 3′ end of the p110-d fragment respectively, using primers gw154-p03 (5′-ATTAAACCAGGAGGAGGAGGAGGAGGACCCCCTGGGGTGGAC-TGCCCCATGGA-3′) (SEQ ID NO: 19) and gwG154-p06 (5′-AGCTCCGTGATGGTGAT-GGTGATGTGCT-CCCTGCCTGTTGTCTTTGGACACGTTGT-3′) (SEQ ID NO: 20).

The p85-iSH2/p110-d fusion protein is assembled in a third PCR reaction by the overlapping linkers at the 3′ end of the iSH2 fragment and the 5′ end of the p110-d fragment, using the above mentioned gwG130-p03 primer and a primer containing an overlapping Histidine tag and the Gateway (Invitrogen) AttB2 recombination sequences (5′-GGGACCACTTTGTA-CAAGAAAGCTGGGTTT-AAGCTCCGTGATGGTGATGGTGATGTGCTCC-3′) (SEQ ID NO: 21).

This final product is recombined in a Gateway (Invitrogen) OR reaction into the donor vector pDONR201 to generate the ORF319 entry clone. This clone is verified by sequencing and used in a Gateway LR reaction to transfer the insert into the Gateway adapted pBlueBac4.5 (Invitrogen) vector for generation of the baculovirus expression vector LR415.

PI3 Kγ BV-950 p110g (D144aa)-C-term His Tag

This construct is obtained from Roger Williams lab, MRC Laboratory of Molecular Biology, Cambridge, UK (November, 2003). Description of the construct in: Pacold M. E. et al. (2000) Cell 103, 931-943.

### 1.3 Protein Expression and Purification

Methods to generate recombinant baculovirus and protein for PI3K isoforms:

The pBlue-Bac4.5 (for a, b, and d isoforms) or pVL1393 (for g) plasmids containing the different PI3 kinase genes are co-transfected with BaculoGold WT genomic DNA (BD Biosciences, Franklin Lakes, N.J., USA) using methods recommended by the vendor. Subsequently, the recombinant baculovirus obtained from the transfection is plaque-purified on Sf9 insect cells to yield several isolates expressing recombinant protein. Positive clones are selected by anti-HIS or anti-isoform antibody western. For PI3K alpha and delta isoforms, a secondary plaque-purification is performed on the first clonal virus stocks of PI3K. Amplification of all baculovirus isolates is performed at low multiplicity of infection (moi) to generate high-titer, low passage stock for protein production. The baculoviruses are designated BV1052 (α) and BV1075 (α), BV949 (β), BV1060 (δ) and BV950 (γ). Protein production involves infection (passage 3 or lower) of suspended Tn5 (Trichoplusia ni) or TiniPro (Expression Systems, LLC, Woodland, Calif., USA) cells in protein-free media at moi of 2-10 for 39-48 hours in 2 l glass Erlenmyer flasks (110 rpm) or wave-bioreactors (22-25 rpm). Initially, 10 l working volume wave-bioreactors are seeded at a density of 3e5 cells/ml at half capacity (5 L). The reactor is rocked at 15 rpm during the cell growth phase for 72 hours, supplemented with 5% oxygen mixed with air (0.2 l per minute). Immediately prior to infection, the wave-reactor cultures are analyzed for density, viability and diluted to approximately 1.5e6 cell/ml. 100-500 ml of high titer, low passage virus is added following 2-4 hours of additional culture. Oxygen is increased to 35% for the 39-48 hour infection period and rocking platform rpm increased to 25. During infection, cells are monitored by Vicell viability analyzer (Beckman Coulter, Inc, Fullerton, Calif., USA) bioprocess for viability, diameter and density. Nova Bioanalyzer (NOVA Biomedical Corp., Waltham, Mass., USA) readings of various parameters and metabolites (pH, O₂ saturation, glucose, etc.) are taken every 12-18 hours until harvest. The wave-bioreactor cells are collected within 40 hours post infection. Cells are collected by centrifugation (4 degrees C. at 1500 rpm), and subsequently maintained on ice during pooling of pellets for lysis and purification. Pellet pools are made with small amounts of cold, un-supplemented Grace's media (w/o protease inhibitors).

### PI3K Alpha Purification Protocol For HTS (BV1052)

PI3K alpha is purified in three chromatographic steps: immobilized metal affinity chromatography on a Ni Sepharose resin (GE Healthcare, belonging to General Electric Company, Fairfield, Conn., USA), gel filtration utilizing a Superdex 200 26/60 column (GE Healthcare), and finally a cation exchange step on a SP-XL column (GE Healthcare). All buffers are chilled to 4° C. and lysis is performed chilled on ice. Column fractionation is performed rapidly at room temperature.

Typically frozen insect cells are lysed in a hypertonic lysis buffer and applied to a prepared IMAC column. The resin is washed with 3-5 column volumes of lysis buffer, followed by 3-5 column volumes wash buffer containing 45 mM imidazole, and the target protein is then eluted with a buffer containing 250 mM imidazole. Fractions are analyzed by Coomassie stained SDS-PAGE gels, and fractions containing target protein are pooled and applied to a prepared GFC column. Fractions from the GFC column are analyzed by Coomassie stained SDS-PAGE gels, and fractions containing target protein are pooled. The pool from the GFC column is diluted into a low salt buffer and applied to a prepared SP-XL column. The column is washed with low salt buffer until a stable A280 baseline absorbance is achieved, and eluted using a 20 column volume gradient from 0 mM NaCl to 500 mM NaCl. Again, fractions from the SP-XL column are analyzed by Coomassie stained SDS-PAGE gels, and fractions containing the target protein are pooled. The final pool is dialyzed into a storage buffer containing 50% glycerol and stored at −20° C. The final pool is assayed for activity in a phosphoinosititol kinase assay.

### PI3K Beta Purification Protocol For HTS (BV949)

PI3K beta is purified in two chromatographic steps: immobilized metal affinity chromatography (IMAC) on a Ni Sepharose resin (GE Healthcare) and gel filtration (GFC) utilizing a Superdex 200 26/60 column (GE Healthcare). All buffers are chilled to 4° C. and lysis is performed chilled on ice. Column fractionation is performed rapidly at room temperature.

Typically frozen insect cells are lysed in a hypertonic lysis buffer and applied to a prepared IMAC column. The resin is washed with 3-5 column volumes of lysis buffer, followed by 3-5 column volumes wash buffer containing 45 mM imidazole, and the target protein is then eluted with a buffer containing 250 mM imidazole. Fractions are analyzed by Coomassie stained SDS-PAGE gels, and fractions containing target protein are pooled and applied to a prepared GFC column. Fractions from the GFC column are analyzed by Coomassie stained SDS-PAGE gels, and fractions containing target protein are pooled. The final pool is dialyzed into a storage buffer containing 50% glycerol and stored at −20° C. The final pool is assayed for activity in the phosphoinostitol kinase assay.

### PI3K Gamma Purification Protocol For HTS (BV950)

PI3K gamma is purified in two chromatographic steps: immobilized metal affinity chromatography (IMAC) on a Ni Sepharose resin (GE Healthcare) and gel filtration (GFC) utilizing a Superdex 200 26/60 column (GE Healthcare). All buffers are chilled to 4° C. and lysis is performed chilled on ice. Column fractionation is performed rapidly at room temperature. Typically frozen insect cells are lysed in a hypertonic lysis buffer and applied to a prepared IMAC column. The resin is washed with 3-5 column volumes of lysis buffer, followed by 3-5 column volumes wash buffer containing 45 mM imidazole, and the target protein is then eluted with a buffer containing 250 mM imidazole. Fractions are analyzed by Coomassie stained SDS-PAGE gels, and fractions containing target protein are pooled and applied to a prepared GFC column. Fractions from the GFC column are analyzed by Coomassie stained SDS-PAGE gels, and fractions containing target protein are pooled. The final pool is dialyzed into a storage buffer containing 50% glycerol and stored at −20° C. The final pool is assayed for activity in the phosphoinostitol kinase assay.

### PI3K Delta Purification Protocol For HTS (BV1060)

PI3K delta is purified in three chromatographic steps: immobilized metal affinity chromatography on a Ni Sepharose resin (GE Healthcare), gel filtration utilizing a Superdex 200 26/60 column (GE Healthcare), and finally a anion exchange step on a Q-HP column (GE Healthcare). All buffers are chilled to 4° C. and lysis is performed chilled on ice. Column fractionation is performed rapidly at room temperature. Typically frozen insect cells are lysed in a hypertonic lysis buffer and applied to a prepared IMAC column. The resin is washed with 3-5 column volumes of lysis buffer, followed by 3-5 column volumes wash buffer containing 45 mM imidazole, and the target protein is then eluted with a buffer containing 250 mM imidazole. Fractions are analyzed by Coomassie stained SDS-PAGE gels, and fractions containing the target protein are pooled and applied to a prepared GFC column. Fractions from the GFC column are analyzed by Coomassie stained SDS-PAGE gels, and fractions containing the target protein are pooled. The pool from the GFC column is diluted into a low salt buffer and applied to a prepared Q-HP column. The column is washed with low salt buffer until a stable A280 baseline absorbance is achieved, and eluted using a 20 column volume gradient from 0 mM NaCl to 500 mM NaCl. Again, fractions from the Q-HP column are analyzed by Coomassie stained SDS-PAGE gels, and fractions containing the target protein are pooled. The final pool is dialyzed into a storage buffer containing 50% glycerol and stored at −20° C. The final pool is assayed for activity in the phosphoinostitol kinase assay.

IC₅₀ is determined by a four parameter curve fitting routine that comes along with “excel fit”. A four parameter logistic equation is used to calculate IC₅₀ values (IDBS XLfit) of the percentage inhibition of each compound at 8 concentrations (usually 10, 3.0, 1.0, 0.3, 0.1, 0.030, 0.010 and 0.003 μM). Alternatively, IC₅₀ values are calculated using idbsXLfit model 204, which is a 4 parameter logistic model.

Yet alternatively, for an ATP depletion assay, compounds of the formula I to be tested are dissolved in DMSO and directly distributed into a white 384-well plate at 0.5 μl per well. To start the reaction, 10 μl of 10 nM PI3 kinase and 5 μg/ml 1-alpha-phosphatidylinositol (PI) are added into each well followed by 10 μl of 2 μM ATP. The reaction is performed until approx 50% of the ATP is depleted, and then stopped by the addition of 20 μl of Kinase-Glo solution (Promega Corp., Madison, Wis., USA). The stopped reaction is incubated for 5 minutes and the remaining ATP is then detected via luminescence. IC₅₀ values are then determined.

Some of the compounds of examples 1-117 show a certain level of selectivity against the different paralogs PI3K α, β, γ and δ.

Suitably, the isoform PI3Kδ e.g as indicated in in vitro and in vivo tests with selectivity of at least 10-fold, and more preferably at least 30-fold against the different paralogs PI3K α and β.

The range of activity, expressed as IC₅₀, in these assays, is preferably between 1 nM and 5000 nM, more preferably between 1 nM and about 1000 nM.

### Cellular Assays

1 Determination of PI3K alpha, PI3K beta and PI3K delta inhibition in Rat1 cells

The efficacy of the compounds in blocking the activation of the PI3K/PKB pathway was demonstrated in cellular settings using a homogeneous sandwich phospho-ELISA based on the ALPHA technology of Perkin Elmer for a sensitive quantification of compound of mediated inhibition of PKB Ser473 phosphorylation in Rat1 cells stably transfected with activated versions of PI3-kinase isoforms alpha, beta or delta:

### 1.1 Cells and Cell Culture Conditions

Rat1 cell lines stably expressing a myr-HA-tagged, constitutively active subunit of the catalytic PI3K class I p110 isoform α, β or δ (addition of a myristylation signal at the N-terminus of p110 isoforms has been shown to lead to constitutive activation of PI3K and corresponding downstream signals, such as phosphorylation of PKB at Ser473) were cultivated in Dulbecco's modified Eagle's medium (DMEM high Glucose, GIBCO, cat. No. 41956-039) supplemented with 10% heat inactivated fetal bovine calf serum (Amimed, cat. No. 2-01F16-I), 1% L-Glutamine (Invitrogen, cat. No. 25030-02), 1% penicillin-streptomycin (GIBCO, cat. No. 15140-114) and 10 μg/ml Puromycine (Sigma, cat. No. P9620).

### 1.2 Compound Treatment of Cells and Preparation of Samples

The compounds tested were prepared as 10 mM stock solutions in DMSO (Merck, #8.02912.2500). Test compounds were prepared in 384 well plates (Greiner PP-Microplate, #781201) in 3-fold serial dilutions in 90% DMSO (Merck, #8.02912.2500) with 8 concentrations starting at 2 mM. Test compounds were prediluted (1:400) in two subsequent 1:20 dilution steps (5 μl+95 μl) from the master plate into starving medium in 384 well plates using a MATRIX PlateMate 2×2 pipettor (384 well-head). 25 μl/well of this 1:400 dilution were then added to the cell culture plate. The final compound dilution of 1:800 resulted in a starting concentration of 2.5 μM for all compounds; the final DMSO concentration was kept constant at 0.125%, also for the control cells (high and low control).

The Rat1-myr-HA-p110 alpha, beta and delta cells were trypsinized and counted with a CASY TT cell counter (Schärfe System GmbH, Reutlingen Germany). Rat1 cells expressing myr-HA-p110 alpha, beta and delta were seeded in 384-well plates at 15,000 cells per well in 50 μl/well complete medium and incubated for 20 h at 37° C., 5% CO₂ until the cell layers reached 80-90% confluency. Test compounds were prediluted (1:400) in two subsequent 1:20 dilution steps (5 μl+95 μl) from the master plate into starving medium in 384 well plates using a MATRIX PlateMate 2×2 pipettor (384 well-head). 25 μl/well of this 1:400 dilution were then added to the cell culture plate. The final compound dilution of 1:800 resulted in a starting concentration of 2.5 μM for all compounds; the final DMSO concentration was kept constant at 0.125%, also for the control cells (high and low control). The final compound dilution of 1:800 resulted in a starting concentration of 2.5 μM for all compounds; the final DMSO concentration was kept constant at 0.125%, also for the control cells (high and low control) to get final compound concentrations of 10, 3.333, 1.111, 0.370, 0.123, 0.041, 0.014, 0.005 μM.

Untreated cells were used as low controls, cells stimulated in absence of compounds were used as high controls. After an incubation of 1 h with compounds, cells were lysed by addition of 15 μl 3× lysis buffer (provided as 5× solution with the SureFire kit) enriched with 0.72% BSA, yielding a total volume of 45 μl cell lysate per well. Cell lysates were either used immediately of stored frozen (in sealed plates) at −20° C. until use. 5 μl of each lysate were transferred to Proxy-plates and mixed with Surefire beads, reaction buffer (containing the appropriate antibodies), activation buffer and dilution buffer according to the instructions of the supplier, yielding a total volume of 12 μl per well (the total final BSA conc. in the assay was 0.1%). After 2 h incubation (with shaking) in the dark at RT, light emission was measured using the EnVision Reader (Perkin Elmer). The difference between high and low controls was taken as 100% and compound effects were expressed as percent inhibition. IC₅₀ values were determined from the dose response curves by graphical extrapolation.

### 2) Determination of Murine B Cell Activation

PI3Kδ has been recognized to modulate B cell function when cells are stimulated through the B cell receptor (BCR) (Okkenhaug et al. Science 297:1031 (2002). For assessing the inhibitory property of compounds on B cell activation, the upregulation of activation markers CD86 and CD69 on murine B cells derived from mouse spleen antibody is measured after stimulation with anti-IgM. CD69 is a well known activation marker for B and T cells (Sancho et al. Trends Immunol. 26:136 (2005). CD86 (also known as B7-2) is primarily expressed on antigen-presenting cells, including B cells. Resting B cells express CD86 at low levels, but upregulate it following stimulation of e.g. the BCR or IL-4 receptor. CD86 on a B cell interacts with CD28 on T cells. This interaction is required for optimal T cell activation and for the generation of an optimal IgG1 response (Carreno et al. Annu Rev Immunol. 20:29 (2002)).

Spleens from Balb/c mice were collected, splenocytes were isolated and washed twice with RPMI containing 10% foetal bovine serum (FBS), 10 mM HEPES, 100 Units/ml penicilline/streptomycine. RPMI supplemented in this way is subsequently referred to as medium. The cells were adjusted to 2.5×10⁶ cells/ml in medium and 200 μl cell suspension (5×10⁶cells) were added to the appropriate wells of 96 well plates.

Then the cells were stimulated by adding 50 μl anti-IgM mAb in medium (final concentration: 30 μg/ml). After incubation for 24 hours at 37° C., the cells were stained with the following antibody cocktails: anti-mouse CD86-FITC, anti-mouse CD69-PerCP-Cy5.5, anti-mouse CD19-PerCP for the assessment of B cells, and anti-mouse CD3-FITC, anti-mouse CD69-PE for the assessment of T cells (2 μl of each antibody/well). After one hour at room temperature (RT) in the dark the cells were transferred to 96 Deepwell plates. The cells were washed once with 1 ml PBS containing 2% FBS and after re-suspension in 200 μl the samples were analyzed on a FACS Calibur flow cytometer. Lymphocytes were gated in the FSC/SSC dot plot according to size and granularity and further analyzed for expression of CD19, CD3 and activation markers (CD86, CD69). Data were calculated from dot blots as percentage of cells positively stained for activation markers within the CD19+ or CD3+ population using BD CellQest Software.

For assessing the inhibitory property of compounds, compounds were first dissolved and diluted in DMSO followed by a 1:50 dilution in medium. Splenocytes from Balb/c mice were isolated, re-suspended and transfered to 96 well plates as described above (200 μl/well). The diluted compounds or solvent were added to the plates (25 μl) and incubated at 37° C. for 1 hour. Then the cultures were stimulated with 25 μl anti-IgM mAb/well (final concentration 30 μg/ml) for 24 hours at 37° C. and stained with anti-mouse CD86-FITC and anti-mouse CD19-PerCP (2 μl of each antibody/well). CD86 expression on CD19 positive B cells was quantified by flow cytometry as described above.

### Biological Data

### Enzymatic Assay

| Example | PI3K alpha (uM) | PI3K delta (uM) |
|---|---|---|
| 1 | 0.294 | 0.0072 |
| 2 | 0.779 | 0.0095 |
| 3 | 0.062 | <0.003 |
| 4 | 1.1585 | 0.009 |
| 5 | 1.3215 | 0.0085 |
| 6 | 0.589 | 0.008625 |
| 7 | 0.712 | 0.006 |
| 8 | 0.268 | 0.0095 |
| 9 | 1.398 | 0.01004 |
| 10 | >9.1 | 0.027 |
| 11 | 1.0735 | 0.028 |
| 12 | 0.4175 | 0.02025 |
| 13 | 1.328 | 0.034 |
| 14 | 0.0785 | <0.003 |
| 15 | 1.2315 | 0.017 |
| 16 | 0.695 | 0.0125 |
| 17 | 0.3525 | 0.006375 |
| 18 | 1.2855 | 0.0421429 |
| 19 | 0.678 | 0.0115 |
| 20 | 2.483 | 0.024 |
| 21 | 2.2676667 | 0.0145 |
| 22 | 0.7895 | 0.0115 |
| 23 | 0.931 | 0.0125 |
| 24 | 0.4 | 0.015 |
| 25 | 0.1835 | 0.007 |
| 26 | 0.6136 | 0.01775 |
| 27 | 0.4035 | 0.0185 |
| 28 | 0.418 | 0.009 |
| 29 | 0.075 | 0.003 |
| 30 | 0.3866667 | 0.0066667 |
| 31 | 0.631 | 0.006 |
| 32 | 0.174 | 0.007 |
| 33 | 0.639 | 0.012 |
| 34 | 0.488 | 0.0225 |
| 35 | 0.262 | 0.007 |
| 36 | 0.21725 | 0.0045 |
| 37 | 0.426 | 0.004 |
| 38 | 0.3725 | 0.007 |
| 39 | 0.77 | 0.0115 |
| 40 | 0.2695 | 0.003 |
| 41 | 0.7075 | 0.0205 |
| 42 | 0.152 | 0.006 |
| 43 | 0.3745 | 0.011 |
| 44 | 0.2825 | <0.003 |
| 45 | 1.1975 | 0.0365 |
| 46 | 5.295 | 0.0235 |
| 47 | >9.1 | 0.027 |
| 48 | 3.7995 | 0.0475 |
| 49 | 8.9455 | 0.0355 |
| 50 | 0.319 | 0.0105 |
| 51 | 0.1175 | 0.0035 |
| 52 | 0.189 | 0.0075 |
| 53 | 0.472 | 0.007 |
| 54 | 0.069 | 0.003 |
| 55 | 0.19275 | 0.00925 |
| 56 | 0.5425 | 0.011 |
| 57 | 0.4615 | 0.02175 |
| 58 | 0.6455 | 0.0055 |
| 59 | 1.7095 | 0.0315 |
| 60 | 0.657 | 0.0195 |
| 61 | 0.39 | 0.003 |
| 62 | 1.151 | 0.007 |
| 63 | 0.505 | 0.006 |
| 64 | 0.583 | 0.011 |
| 65 | 0.64 | 0.01 |
| 66 | 0.763 | 0.0095 |
| 67 | 0.306 | 0.007 |
| 68 | 0.097 | 0.003 |
| 69 | 0.88 | 0.008 |
| 70 | 1.026 | 0.03 |
| 71 | 0.5505 | 0.008 |
| 72 | 0.0695 | 0.003 |
| 73 | 0.0605 | <0.003 |
| 74 | 0.1496667 | 0.005 |
| 75 | 0.3195 | 0.012 |
| 76 | 0.5775 | 0.0235 |
| 77 | 0.573 | 0.014 |
| 78 | 0.123 | 0.0045 |
| 79 | 0.3595 | 0.01 |
| 80 | 0.717 | 0.0105 |
| 81 | 0.401 | 0.003625 |
| 82 | 1.334 | 0.0215 |
| 83 | 0.904 | 0.021 |
| 84 | 0.5585 | 0.0095 |
| 85 | 0.793 | 0.007 |
| 86 | 0.3805 | 0.0155 |
| 87 | 0.126 | <0.003 |
| 88 | 0.1 | 0.002675 |
| 89 | 0.099 | 0.004 |
| 90 | 0.181 | 0.00575 |
| 91 | 0.1465 | <0.003 |
| 92 | 0.1955 | 0.00375 |
| 93 | 0.12 | <0.003 |
| 94 | 0.174 | 0.006 |
| 95 | 0.281 | 0.009 |
| 96 | 0.316 | 0.006 |
| 97 | 0.463 | 0.023 |
| 98 | 0.1015 | 0.0035 |
| 99 | 0.237 | 0.0045 |
| 100 | 0.188 | 0.005 |
| 101 | 1.271 | 0.016 |
| 102 | 0.77 | 0.011 |
| 103 | 2.121 | 0.008 |
| 104 | 3.293 | 0.038 |
| 105 | 0.72 | 0.007 |
| 106 | 0.44 | 0.005 |
| 107 | 0.716 | 0.005 |
| 108 | 0.158 | 0.007 |
| 109 | 0.435 | 0.014 |
| 110 | 0.366 | 0.008 |
| 111 | 1.357 | 0.01 |
| 112 | 2.316 | 0.049 |
| 113 | 0.979 | 0.018 |
| 114 | 0.071 | 0.003 |
| 115 | 0.888 | 0.0425 |
| 116 | 0.306 | 0.014 |
| 117 | 0.238 | 0.009 |

### Cellular Assay

| | Cell PI3Kd/ | mCD86/ |
|---|---|---|
| | IC50 | IC50 CD86 |
| Example | [umol I-1] | [nmol I-1] |
| 1 | 0.02735 | 69.5 |
| 2 | 0.0275 | 69.5 |
| 7 | 0.083 | 20.15 |
| 12 | 0.0535 | 107 |
| 44 | 0.037 | 86.5 |
| 75 | 0.014 | 54 |
| 81 | 0.0195 | 79 |
| 91 | 0.0323333 | 44 |
| 110 | 0.103 | 155 |
| 117 | 0.043 | 101 |

1-16. (canceled)

17. A substituted quinazoline derivative of the formula (I) and/or tautomers and/or N-oxides and/or pharmaceutically acceptable salts thereof, 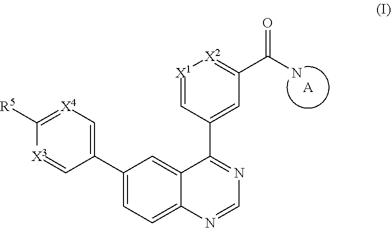
wherein
A is a saturated, 5-8 membered mono- or 6-12 membered bicyclic fused, bicyclic bridged or bicyclic spiro heterocyclic ring optionally containing 1-2 additional heteroatoms selected from N, O or S, wherein the heterocyclic ring is unsubstituted or substituted by 1-4 substituents selected from hydroxy, halo, C₁-C₇-alkyl, C₁-C₇-alkyl-carbonyl, halo-C₁-C₇-alkyl, halo-C₁-C₇-alkyl-carbonyl, C₁-C₇-alkoxy-carbonyl, and oxo;
X¹ is CH, N or CR;
X² is CH, N or CR, wherein R is independently selected from the group consisting of halogen, halo-C₁-C₇-alkyl, C₁-C₇-alkyl, and C₁-C₇-alkoxy;
X³ is CH, N or CR³, wherein R³ is cyano, nitro, halogen, halo-C₁-C₇-alkyl, C₁-C₇-alkoxy, C₁-C₁₀-cycloalkyl-oxy, phenyl-oxy, benzyl-oxy, C₁-C₇-alkoxy-C₁-C₇-alkoxy, carboxyl, C₁-C₇-alkoxy-carbonyl, amino-carbonyl, N—C₁-C₇-alkyl-amino-carbonyl, amino-carbonyl, amino-sulfonyl, N—C₁-C₇-alkyl-amino-sulfonyl, N,N-di-C₁-C₇-alkyl-amino-sulfonyl, 1-pyrrolidino-sulfonyl, 4-morpholino-sulfonyl, C₁-C₇-alkyl-sulfonyl, or C₁-C₇-alkyl-sulfonyl-amino-;
X⁴ is CH, N, CR⁴ wherein R⁴ is trifluoromethyl; and
R⁵ is hydrogen, halogen, hydroxy, C₁-C₇-alkoxy, halo-C₁-C₇-alkyl, halo-C₁-C₇-alkyl-oxy, amino, N—C₁-C₇-alkyl-amino, C₁-C₇-alkyl-carbonyl, C₁-C₇-alkyl-carbonyl-amino, amino-sulfonyl, C₁-C₇-alkyl-sulfonyl-amino, 1-pyrrolidinyl or 1-piperazinyl, with the proviso that, if X⁴ is CH, then R³ and R⁵ are not both methoxy.

18. A compound according to claim 17, wherein
A is a saturated heterocycle selected from the group consisting of 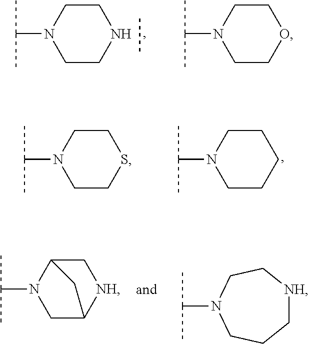
each of which is unsubstituted or substituted by 1-4 substituents selected from the group consisting of hydroxy, halo, C₁-C₇-alkyl, C₁-C₇-alkyl-carbonyl, halo-halo-C₁-C₇-alkyl-carbonyl, C₁-C₇-alkoxy-carbonyl and oxo.

19. A compound according to claim 17, wherein
X⁴ is N;
R⁵ is selected from the group consisting of C₁-C₇-alkyl, C₁-C₇-alkoxy, halo-C₁-C₇-alkyl-oxy, amino, N—C₁-C₇-alkyl-amino, 1-pyrrolidinyl, and 1-piperazinyl; and
X³ is CH or CR³, wherein R³ is selected from the group consisting of cyano, halogen, halo-C₁-C₇-alkyl and C₁-C₇-alkyl.

20. A compound according to claim 17, wherein
X³ and X⁴ are N; and
R⁵ is selected from the group consisting of C₁-C₇-alkyl, C₁-C₇-alkoxy, halo-C₁-C₇-alkyl-oxy, amino, N—C₁-C₇-alkyl-amino, 1-pyrrolidinyl and 1-piperazinyl.

21. A compound according to claim 17, wherein
X³ is CR³, wherein R³ is selected from the group consisting of N,N-di-C₁-C₇-alkyl-amino-carbonyl, N,N-di-C₁-C₇-alkyl-amino-sulfonyl, 1-pyrrolidino-sulfonyl, 4-morpholino-sulfonyl, C₁-C₇-alkyl-sulfonyl, and C₁-C₇-alkyl-sulfonyl-amino;
X⁴ is N; and
R⁵ is hydrogen.

22. A compound according to claim 17, wherein
X³ is CR³ wherein R³ is selected from the group consisting of cyano, halogen, halo-C₁-C₇-alkyl, and C₁-C₇-alkyl;
X⁴ is CH; and
R⁵ is selected from the group consisting of C₁-C₇-alkyl, C₁-C₇-alkoxy, halo-C₁-C₇-alkyl-oxy, amino, N—C₁-C₇-alkyl-amino, N,N-di-C₁-C₇-alkyl-amino.

23. A compound according to claim 17, wherein
X³ is CH;
X⁴ is CR⁴;
R⁴ is trifluoromethyl; and
R⁵ is amino-sulfonyl or C₁-C₇-alkyl-sulfonyl-amino.

24. A compound according to claim 17, wherein
A is a saturated heterocycle selected from 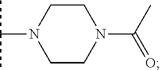
X¹ is CR¹ wherein R¹ fluoro;
X² is CH;
X³ is CH or CR³, wherein R³ is cyano;
X⁴ is N; and
R⁵ is methoxy.

25. A compound according to claim 17, wherein
A is a saturated heterocycle selected from 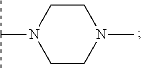
X¹ is CH;
X² is CH;
X³ is N;
X⁴ is N; and
R⁵ is methoxy.

26. A pharmaceutical composition comprising a therapeutically effective amount of a compound of formula (I) as defined in claim 17, and one or more pharmaceutically acceptable carriers.

27. A method of modulating the activity of at least one PI3K enzyme in a subject, comprising the step of administering to a subject a therapeutically effective amount of a compound of formula (I) as defined in claim 17.

28. The method of claim 27, wherein the PI3K enzyme is PI3Kδ.

29. A method for the treatment of a disorder or a disease mediated by the PI3K enzymes in a subject, the method comprising the step of administering to a subject a therapeutically effective amount of a compound of formula (I) as defined in claim 17.

30. A method according to claim 29, wherein the disorder or a disease is selected from autoimmune disorders, inflammatory diseases, allergic diseases, airway diseases, transplant rejection, cancers.

31. The method of claim 30, wherein the airway disease is asthma and COPD and the cancer is selected from cancers of hematopoietic origin or solid tumors.


SEQUENCE LISTING (21 sequences)

SEQ ID NO: 1 - DNA, 28 bp, Artificial
  Primer
cgagaatatgatagattatatgaagaat

SEQ ID NO: 2 - DNA, 30 bp, Artificial
  Primer
tggtttaatgctgttcatacgtttgtcaat

SEQ ID NO: 3 - DNA, 76 bp, Artificial
  Primer
gggacaagtttgtacaaaaaagcaggctacgaaggagatatacatatgcgagaatatgatagattatatgaagaat

SEQ ID NO: 4 - DNA, 66 bp, Artificial
  Primer
taccataattccaccaccaccaccggaaattccccctggtttaatgctgttcatacgtttgtcaat

SEQ ID NO: 5 - DNA, 26 bp, Artificial
  Primer
ctagtggaatgtttactaccaaatgg

SEQ ID NO: 6 - DNA, 26 bp, Artificial
  Primer
gttcaatgcatgctgtttaattgtgt

SEQ ID NO: 7 - DNA, 63 bp, Artificial
  Primer
gggggaatttccggtggtggtggtggaattatggtactagtggaatgtttactaccaaatgga

SEQ ID NO: 8 - DNA, 56 bp, Artificial
  Primer
agctccgtgatggtgatggtgatgtgctccgttcaatgcatgctgtttaattgtgt

SEQ ID NO: 9 - DNA, 61 bp, Artificial
  Primer
gggaccactttgtacaagaaagctgggtttaagctccgtgatggtgatggtgatgtgctcc

SEQ ID NO: 10 - DNA, 42 bp, Artificial
  Primer
gctagcatgcgagaatatgatagattatatgaagaatatacc

SEQ ID NO: 11 - DNA, 45 bp, Artificial
  Primer
gcctccaccacctccgcctggtttaatgctgttcatacgtttgtc

SEQ ID NO: 12 - DNA, 42 bp, Artificial
  Primer
tactagtccgcctccaccacctccgcctccaccacctccgcc

SEQ ID NO: 13 - DNA, 54 bp, Artificial
  Primer
actgaagcatcctcctcctcctcctcctggtttaatgctgttcatacgtttgtc

SEQ ID NO: 14 - DNA, 57 bp, Artificial
  Primer
agctccgtgatggtgatggtgatgtgctccagatctgtagtctttccgaactgtgtg

SEQ ID NO: 15 - DNA, 61 bp, Artificial; identical to SEQ ID NO: 9
  Primer

SEQ ID NO: 16 - DNA, 45 bp, Artificial
  Primer
tcctcctcctcctcctcctggtttaatgctgttcatacgtttgtc

SEQ ID NO: 17 - DNA, 26 bp, Artificial
  Primer
atgccccctggggtggactgccccat

SEQ ID NO: 18 - DNA, 26 bp, Artificial
  Primer
ctactgcctgttgtctttggacacgt

SEQ ID NO: 19 - DNA, 53 bp, Artificial
  Primer
attaaaccaggaggaggaggaggaggaccccctggggtggactgccccatgga

SEQ ID NO: 20 - DNA, 56 bp, Artificial
  Primer
agctccgtgatggtgatggtgatgtgctccctgcctgttgtctttggacacgttgt

SEQ ID NO: 21 - DNA, 61 bp, Artificial; identical to SEQ ID NO: 9
  Primer